SEQUENCE LISTING (63 sequences)

SEQ ID NO: 1 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 2 - DNA, 1257 bp, Homo sapiens
atgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataa

SEQ ID NO: 3 - protein, 341 aa, Cowpox virus
MDIFREIASSMKGENVFISPPSISSVLTILYYGANGSTAEQLSKYVEKEADKNKDDISFKSMNKVYGRYSAVFKDSFLRKIGDNFQTVDFTDCRTVDAINKCVDIFTEGKINPLLDEPLSPDTCLLAISAVYFKAKWLMPFEKEFTSDYPFYVSPTEMVDVSMMSMYGEAFNHASVKESFGNFSIIELPYVGDTSMVVILPDNIDGLESIEQNLTDTNFKKWCDSMDAMFIDVHIPKFKVTGSYNLVDALVKLGLTEVFGSTGDYSNMCNSDVSVDAMIHKTYIDVNEEYTEAAAATCALVADCASTVTNEFCADHPFIYVIRHVDGKILFVGRYCSPTTN

SEQ ID NO: 4 - DNA, 1026 bp, Cowpox virus
atggatatcttcagggaaatcgcatcttctatgaaaggagagaatgtattcatttctccaccgtcaatctcgtcagtattgacaatactgtattatggagctaatggatccactgctgaacagctatcaaaatatgtagaaaaggaggcggacaagaataaggatgatatctcattcaagtccatgaataaagtatatgggcgatattctgcagtgtttaaagattcctttttgagaaaaattggagataatttccaaactgttgacttcactgattgtcgcactgtagatgcgatcaacaagtgtgttgatatcttcactgaggggaaaattaatccactattggatgaaccattgtctccagatacctgtctcctagcaattagtgccgtatactttaaagcaaaatggttgatgccatttgaaaaggaatttaccagtgattatcccttttacgtatctccaacggaaatggtagatgtaagtatgatgtctatgtacggcgaggcatttaatcacgcatctgtaaaagaatcattcggcaacttttcaatcatagaactgccatatgttggagatactagtatggtggtaattcttccagacaatattgatggactagaatccatagaacaaaatctaacagatacaaattttaagaaatggtgtgactctatggatgctatgtttatcgatgtgcacattcccaagtttaaggtaacaggctcgtataatctggtggatgcgctagtaaagttgggactgacagaggtgttcggttcaactggagattatagcaatatgtgtaattcagatgtgagtgtcgacgctatgatccacaaaacgtatatagatgtcaatgaagagtatacagaagcagctgcagcaacttgtgcgctggtggcagactgtgcatcaacagttacaaatgagttctgtgcagatcatccgttcatctatgtgattaggcatgtcgatggcaaaattcttttcgttggtagatattgctctccaacaactaattaa

SEQ ID NO: 5 - protein, 391 aa, Arabidopsis thaliana
MDVRESISLQNQVSMNLAKHVITTVSQNSNVIFSPASINVVLSIIAAGSAGATKDQILSFLKFSSTDQLNSFSSEIVSAVLADGSANGGPKLSVANGAWIDKSLSFKPSFKQLLEDSYKAASNQADFQSKAVEVIAEVNSWAEKETNGLITEVLPEGSADSMTKLIFANALYFKGTWNEKFDESLTQEGEFHLLDGNKVTAPFMTSKKKQYVSAYDGFKVLGLPYLQGQDKRQFSMYFYLPDANNGLSDLLDKIVSTPGFLDNHIPRRQVKVREFKIPKFKFSFGFDASNVLKGLGLTSPFSGEEGLTEMVESPEMGKNLCVSNIFHKACIEVNEEGTEAAAASAGVIKLRGLLMEEDEIDFVADHPFLLVVTENITGVVLFIGQVVDPLH

SEQ ID NO: 6 - DNA, 1446 bp, Arabidopsis thaliana
cgtcttctcctaaacccagcaaattcgtttaccagtcatcaccaccacaacctccggcgaaaatggacgtgcgtgaatcaatctcactgcaaaaccaagtctccatgaatctcgcaaaacacgtaatcaccaccgtctctcaaaactccaacgtcatcttctcaccggcttcaatcaacgtcgtactcagtataatcgccgctggatccgccggcgctaccaaagatcagatcctctcgtttctcaaattctcttccactgatcaacttaattcattctcttccgaaatcgtctccgctgttctcgctgacggtagtgctaacggtggtcctaagctctcggtggctaatggcgcctggatcgataagtctctctcctttaaaccttcctttaaacagctcttggaagattcgtataaagctgcttcgaatcaagctgattttcaatcgaaggctgtggaggtgattgctgaagtgaattcatgggctgaaaaggagacaaatggtctcatcactgaggttcttccagaaggatcagctgatagtatgaccaaactgatatttgcaaatgcattgtacttcaagggaacatggaacgagaaattcgatgagtcgttaacacaagaaggcgagtttcaccttcttgacggtaacaaagtgactgcaccattcatgaccagcaagaagaaacaatacgtaagtgcttacgatggtttcaaagttttgggacttccttacttacaaggacaggataagcgacaattctccatgtacttttatcttcccgatgcaaacaacggactgtctgatcttctggacaaaatagtttccactcctgggttcttagacaaccacatcccacgcagacaagttaaagtccgcgaattcaagattccaaagtttaaattctctttcgggttcgatgcttcaaatgttttaaaaggattgggactgacttcgcctttcagcggtgaagaaggtttaactgagatggttgaatctcctgagatggggaagaatctatgcgtatcgaacattttccataaagcgtgtatcgaagtgaatgaagaaggaacagaagctgcagctgcatcagctggagttataaagctaagaggattgcttatggaggaagatgaaatagattttgttgcagaccatccgtttctattggtggtcacagagaacataacaggagtggttctgttcattggccaagttgttgatccgttgcattaatctaaagctaatgtggaagtttttggttttacttaaaataaatgagtcattggttttgaggactcatctttatgtaacatcctttgtcttgactctttgatgtgtgtaagaataatagtgatacataacagcttttcttctgtatttggatcacatgtactgaactgatagacatacatacatgtgatgcatcttaatgattcactgt

SEQ ID NO: 7 - protein, 376 aa, Homo sapiens
METLSNASGTFAIRLLKILCQDNPSHNVFCSPVSISSALAMVLLGAKGNTATQMAQALSLNTEEDIHRAFQSLLTEVNKAGTQYLLRTANRLFGEKTCQFLSTFKESCLQFYHAELKELSFIRAAEESRKHINTWVSKKTEGKIEELLPGSSIDAETRLVLVNAIYFKGKWNEPFDETYTREMPFKINQEEQRPVQMMYQEATFKLAHVGEVRAQLLELPYARKELSLLVLLPDDGVELSTVEKSLTFEKLTAWTKPDCMKSTEVEVLLPKFKLQEDYDMESVLRHLGIVDAFQQGKADLSAMSAERDLCLSKFVHKSFVEVNEEGTEAAAASSCFVVAECCMESGPRFCADHPFLFFIRHNRANSILFCGRFSSP

SEQ ID NO: 8 - DNA, 1068 bp, Homo sapiens
atggaaactctttctaatgcaagtggtacttttgccatacgccttttaaagatactgtgtcaagataacccttcgcacaacgtgttctgttctcctgtgagcatctcctctgccctggccatggttctcctaggggcaaagggaaacaccgcaacccagatggcccaggcactgtctttaaacacagaggaagacattcatcgggctttccagtcgcttctcactgaagtgaacaaggctggcacacagtacctgctgagaacggccaacaggctctttggagagaaaacttgtcagttcctctcaacgtttaaggaatcctgtcttcaattctaccatgctgagctgaaggagctttcctttatcagagctgcagaagagtccaggaaacacatcaacacctgggtctcaaaaaagaccgaaggtaaaattgaagagttgttgccgggtagctcaattgatgcagaaaccaggctggttcttgtcaatgccatctacttcaaaggaaagtggaatgaaccgtttgacgaaacatacacaagggaaatgccctttaaaataaaccaggaggagcaaaggccagtgcagatgatgtatcaggaggccacgtttaagctcgcccacgtgggcgaggtgcgcgcgcagctgctggagctgccctacgccaggaaggagctgagcctgctggtgctgctgcctgacgacggcgtggagctcagcaagagtactgaggttgaagttctccttccaaaatttaaactacaagaggattatgacatggaatctgtgcttcggcatttgggaattgttgatgccttccaacagggcaaggctgacttgtcggcaatgtcagcggagagagacctgtgtctgtccaagttcgtgcacaagagttttgtggaggtgaatgaagaaggcaccgaggcagcggcagcgtcgagctgctttgtagttgcagagtgctgcatggaatctggccccaggttctgtgctgaccaccctttccttttcttcatcaggcacaacagagccaacagcattctgttctgtggcaggttctcatcgcca

SEQ ID NO: 9 - protein, 95 aa, Homo sapiens [sequence omitted]

SEQ ID NO: 10 - protein, 423 aa, Homo sapiens
MERMLPLLALGLLAAGFCPAVLCHPNSPLDEENLTQENQDRGTHVDLGLASANVDFAFSLYKQLVLKAPDKNVIFSPLSISTALAFLSLGAHNTTLTEILKGLKFNLTETSEAEIHQSFQHLLRTLNQSSDELQLSMGNAMFVKEQLSLLDRFTEDAKRLYGSEAFATDFQDSAAAKKLINDYVKNGTRGKITDLIKDLDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKAVLDVFEEGTEASAATAVKITLLSALVETRTIVRFNRPFLMIIVPTDTQNIFFMSKVTNPKQA

SEQ ID NO: 11 - DNA, 1272 bp, Homo sapiens
atggagagaatgttacctctcctggctctggggctcttggcggctgggttctgccctgctgtcctctgccaccctaacagcccacttgacgaggagaatctgacccaggagaaccaagaccgagggacacacgtggacctcggattagcctccgccaacgtggacttcgctttcagcctgtacaagcagttagtcctgaaggcccctgataagaatgtcatcttctccccactgagcatctccaccgccttggccttcctgtctctgggggcccataataccaccctgacagagattctcaaaggcctcaagttcaacctcacggagacttctgaggcagaaattcaccagagcttccagcacctcctgcgcaccctcaatcagtccagcgatgagctgcagctgagtatgggaaatgccatgtttgtcaaagagcaactcagtctgctggacaggttcacggaggatgccaagaggctgtatggctccgaggcctttgccactgactttcaggactcagctgcagctaagaagctcatcaacgactacgtgaagaatggaactagggggaaaatcacagatctgatcaaggaccttgactcgcagacaatgatggtcctggtgaattacatcttctttaaagccaaatgggagatgccctttgacccccaagatactcatcagtcaaggttctacttgagcaagaaaaagtgggtaatggtgcccatgatgagtttgcatcacctgactataccttacttccgggacgaggagctgtcctgcaccgtggtggagctgaagtacacaggcaatgccagcgcactcttcatcctccctgatcaagacaagatggaggaagtggaagccatgctgctcccagagaccctgaagcggtggagagactctctggagttcagagagataggtgagctctacctgccaaagttttccatctcgagggactataacctgaacgacatacttctccagctgggcattgaggaagccttcaccagcaaggctgacctgtcagggatcacaggggccaggaacctagcagtctcccaggtggtccataaggctgtgcttgatgtatttgaggagggcacagaagcatctgctgccacagcagtcaaaatcaccctcctttctgcattagtggagacaaggaccattgtgcgtttcaacaggcccttcctgatgatcattgtccctacagacacccagaacatcttcttcatgagcaaagtcaccaatcccaagcaagcctag

SEQ ID NO: 12 - protein, 427 aa, Homo sapiens
MHLIDYLLLLLVGLLALSHGQLHVEHDGESCSNSSHQQILETGEGSPSLKIAPANADFAFRFYYLIASETPGKNIFFSPLSISAAYAMLSLGACSHSRSQILEGLGFNLTELSESDVHRGFQHLLHTLNLPGHGLETRVGSALFLSHNLKFLAKFLNDTMAVYEAKLFHTNFYDTVGTIQLINDHVKKETRGKIVDLVSELKKDVLMVLVNYIYFKALWEKPFISSRTTPKDFYVDENTTVRVPMMLQDQEHHWYLHDRYLPCSVLRMDYKGDATVFFILPNQGKMREIEEVLTPEMLMRWNNLLRKRNFYKKLELHLPKFSISGSYVLDQILPRLGFTDLFSKWADLSGITKQQKLEASKSFHKATLDVDEAGTEAAAATSFAIKFFSAQTNRHILRFNRPFLVVIFSTSTQSVLFLGKVVDPTKP

SEQ ID NO: 13 - DNA, 1284 bp, Homo sapiens
atgcatcttatcgactacctgctcctcctgctggttggactactggccctttctcatggccagctgcacgttgagcatgatggtgagagttgcagtaacagctcccaccagcagattctggagacaggtgagggctcccccagcctcaagatagcccctgccaatgctgactttgccttccgcttctactacctgatcgcttcggagaccccggggaagaacatctttttctccccgctgagcatctcggcggcctacgccatgctttccctgggggcctgctcacacagccgcagccagatccttgagggcctgggcttcaacctcaccgagctgtctgagtccgatgtccataggggcttccagcacctcctgcacactctcaacctccccggccatgggctggaaacacgcgtgggcagtgctctgttcctgagccacaacctgaagttccttgcaaaattcctgaatgacaccatggccgtctatgaggctaaactcttccacaccaacttctacgacactgtgggcacaatccagcttatcaacgaccacgtcaagaaggaaactcgagggaagattgtggatttggtcagtgagctcaagaaggacgtcttgatggtgctggtgaattacatttacttcaaagccctgtgggagaaaccattcatttcctcaaggaccactcccaaagacttctatgttgatgagaacacaacagtccgggtgcccatgatgctgcaggaccaggagcatcactggtatcttcatgacagatacttgccctgctcggtgctacggatggattacaaaggagacgcaaccgtgtttttcattctccctaaccaaggcaaaatgagggagattgaagaggttctgactccagagatgctaatgaggtggaacaacttgttgcggaagaggaatttttacaagaagctagagttgcatcttcccaagttctccatttctggctcctatgtattagatcagattttgcccaggctgggcttcacggatctgttctccaagtgggctgacttatccggcatcaccaaacagcaaaaactggaggcatccaaaagtttccacaaggccaccttggacgtggatgaggctggcaccgaggctgcagcagccaccagcttcgcgatcaaattcttctctgcccagaccaatcgccacatcctgcgattcaaccggcccttccttgtggtgatcttttccaccagcacccagagtgtcctctttctgggcaaggtcgtcgaccccacgaaaccatag

SEQ ID NO: 14 - protein, 427 aa, Homo sapiens; identical to SEQ ID NO: 12

SEQ ID NO: 15 - DNA, 1284 bp, Homo sapiens; identical to SEQ ID NO: 13

SEQ ID NO: 16 - protein, 464 aa, Homo sapiens
MPGDPEKPPPGTHSWYRAALTEGQGLLAANPGLRVQRMHLIDYLLLLLVGLLALSHGQLHVEHDGESCSNSSHQQILETGEGSPSLKIAPANADFAFRFYYLIASETPGKNIFFSPLSISAAYAMLSLGACSHSRSQILEGLGFNLTELSESDVHRGFQHLLHTLNLPGHGLETRVGSALFLSHNLKFLAKFLNDTMAVYEAKLFHTNFYDTVGTIQLINDHVKKETRGKIVDLVSELKKDVLMVLVNYIYFKALWEKPFISSRTTPKDFYVDENTTVRVPMMLQDQEHHWYLHDRYLPCSVLRMDYKGDATVFFILPNQGKMREIEEVLTPEMLMRWNNLLRKRNFYKKLELHLPKFSISGSYVLDQILPRLGFTDLFSKWADLSGITKQQKLEASKSFHKATLDVDEAGTEAAAATSFAIKFFSAQTNRHILRFNRPFLVVIFSTSTQSVLFLGKVVDPTKP

SEQ ID NO: 17 - DNA, 1395 bp, Homo sapiens
atgcccggagacccagaaaagcctcccccagggacacacagctggtacagggcggcactgactgagggccaaggtcttctggctgccaatccaggcctgagagtgcagaggatgcatcttatcgactacctgctcctcctgctggttggactactggccctttctcatggccagctgcacgttgagcatgatggtgagagttgcagtaacagctcccaccagcagattctggagacaggtgagggctcccccagcctcaagatagcccctgccaatgctgactttgccttccgcttctactacctgatcgcttcggagaccccggggaagaacatctttttctccccgctgagcatctcggcggcctacgccatgctttccctgggggcctgctcacacagccgcagccagatccttgagggcctgggcttcaacctcaccgagctgtctgagtccgatgtccataggggcttccagcacctcctgcacactctcaacctccccggccatgggctggaaacacgcgtgggcagtgctctgttcctgagccacaacctgaagttccttgcaaaattcctgaatgacaccatggccgtctatgaggctaaactcttccacaccaacttctacgacactgtgggcacaatccagcttatcaacgaccacgtcaagaaggaaactcgagggaagattgtggatttggtcagtgagctcaagaaggacgtcttgatggtgctggtgaattacatttacttcaaagccctgtgggagaaaccattcatttcctcaaggaccactcccaaagacttctatgttgatgagaacacaacagtccgggtgcccatgatgctgcaggaccaggagcatcactggtatcttcatgacagatacttgccctgctcggtgctacggatggattacaaaggagacgcaaccgtgtttttcattctccctaaccaaggcaaaatgagggagattgaagaggttctgactccagagatgctaatgaggtggaacaacttgttgcggaagaggaatttttacaagaagctagagttgcatcttcccaagttctccatttctggctcctatgtattagatcagattttgcccaggctgggcttcacggatctgttctccaagtgggctgacttatccggcatcaccaaacagcaaaaactggaggcatccaaaagtttccacaaggccaccttggacgtggatgaggctggcaccgaggctgcagcagccaccagcttcgcgatcaaattcttctctgcccagaccaatcgccacatcctgcgattcaaccggcccttccttgtggtgatcttttccaccagcacccagagtgtcctctttctgggcaaggtcgtcgaccccacgaaaccatag

SEQ ID NO: 18 - protein, 406 aa, Homo sapiens
MQLFLLLCLVLLSPQGASLHRHHPREMKKRVEDLHVGATVAPSSRRDFTFDLYRALASAAPSQSIFFSPVSISMSLAMLSLGAGSSTKMQILEGLGLNLQKSSEKELHRGFQQLLQELNQPRDGFQLSLGNALFTDLVVDLQDTFVSAMKTLYLADTFPTNFRDSAGAMKQINDYVAKQTKGKIVDLLKNLDSNAVVIMVNYIFFKAKWETSFNHKGTQEQDFYVTSETVVRVPMMSREDQYHYLLDRNLSCRVVGVPYQGNATALFILPSEGKMQQVENGLSEKTLRKWLKMFKKRQLELYLPKFSIEGSYQLEKVLPSLGISNVFTSHADLSGISNHSNIQVSEMVHKAVVEVDESGTRAAAATGTIFTFRSARLNSQRLVFNRPFLMFIVDNNILFLGKVNRP

SEQ ID NO: 19 - DNA, 1221 bp, Homo sapiens
atgcagctcttcctcctcttgtgcctggtgcttctcagccctcagggggcctcccttcaccgccaccacccccgggagatgaagaagagagtcgaggacctccatgtaggtgccacggtggcccccagcagcagaagggactttacctttgacctctacagggccttggcttccgctgcccccagccagagcatcttcttctcccctgtgagcatctccatgagcctggccatgctctccctgggggctgggtccagcacaaagatgcagatcctggagggcctgggcctcaacctccagaaaagctcagagaaggagctgcacagaggctttcagcagctccttcaggaactcaaccagcccagagatggcttccagctgagcctcggcaatgcccttttcaccgacctggtggtagacctgcaggacaccttcgtaagtgccatgaagacgctgtacctggcagacactttccctaccaactttagggactctgcaggggccatgaagcagatcaatgattatgtggcaaagcaaacgaagggcaagattgtggacttgcttaagaacctcgatagcaatgcggtcgtgatcatggtgaattacatcttctttaaagctaagtgggagacaagcttcaaccacaaaggcacccaagagcaagacttctacgtgacctcggagactgtggtgcgggtacccatgatgagccgcgaggatcagtatcactacctcctggaccggaacctctcctgcagggtggtgggggtcccctaccaaggcaatgccacggctttgttcattctccccagtgagggaaagatgcagcaggtggagaatggactgagtgagaaaacgctgaggaagtggcttaagatgttcaaaaagaggcagctcgagctttaccttcccaaattctccattgagggctcctatcagctggagaaagtcctccccagtctggggatcagtaacgtcttcacctcccatgctgatctgtccggcatcagcaaccactcaaatatccaggtgtctgagatggtgcacaaagctgtggtggaggtggacgagtcgggaaccagagcagcggcagccacggggacaatattcactttcaggtcggcccgcctgaactctcagaggctagtgttcaacaggccctttctgatgttcattgtggataacaacatcctcttccttggcaaagtgaaccgcccctga

SEQ ID NO: 20 - protein, 286 aa, Homo sapiens
MGSALFVKKELQLQANFLGNVKRLYEAEVFSTDFSNPSIAQARINSHVKKKTQGKVVDIIQGLDLLTAMVLVNHIFFKAKWEKPFHPEYTRKNFPFLVGEQVTVHVPMMHQKEQFAFGVDTELNCFVLQMDYKGDAVAFFVLPSKGKMRQLEQALSARTLRKWSHSLQKRWIEVFIPRFSISASYNLETILPKMGIQNVFDKNADFSGIAKRDSLQVSKATHKAVLDVSEEGTEATAATTTKFIVRSKDGPSYFTVSFNRTFLMMITNKATDGILFLGKVENPTKS

SEQ ID NO: 21 - DNA, 861 bp, Homo sapiens
atgggaagtgccctcttcgtcaagaaggagctgcagctgcaggcaaatttcttgggcaatgtcaagaggctgtatgaagcagaagtcttttctacagatttctccaacccctccattgcccaggcgaggatcaacagccatgtgaaaaagaagacccaagggaaggttgtagacataatccaaggccttgaccttctgacggccatggttctggtgaaccacattttctttaaagccaagtgggagaagccctttcaccctgaatatacaagaaagaacttcccattcctggtgggcgagcaggtcactgtgcatgtccccatgatgcaccagaaagagcagttcgcttttggggtggatacagagctgaactgctttgtgctgcagatggattacaagggagatgccgtggccttctttgtcctccctagcaagggcaagatgaggcaactggaacaggccttgtcagccagaacactgagaaagtggagccactcactccagaaaaggtggatagaggtgttcatccccagattttccatttctgcctcctacaatctggaaaccatcctcccgaagatgggcatccaaaatgtctttgacaaaaatgctgatttttctggaattgcaaagagagactccctgcaggtttctaaagcaacccacaaggctgtgctggatgtcagtgaagagggcactgaggccacagcagctaccaccaccaagttcatagtccgatcgaaggatggcccctcttacttcactgtctccttcaataggaccttcctgatgatgattacaaataaagccacagacggtattctctttctagggaaagtggaaaatcccactaaatcctag

SEQ ID NO: 22 - protein, 399 aa, Homo sapiens
MRSAGGRGEIKVRRELQPSKQVSGLTNHARTGQEKRNLQRLVLETPSQNIFFSPVSVSTSLAMLSLGAHSVTKTQILQGLGFNLTHTPESAIHQGFQHLVHSLTVPSKDLTLKMGSALFVKKELQLQANFLGNVKRLYEAEVFSTDFSNPSIAQARINSHVKKKTQGKVVDIIQGLDLLTAMVLVNHIFFKAKWEKPFHPEYTRKNFPFLVGEQVTVHVPMMHQKEQFAFGVDTELNCFVLQMDYKGDAVAFFVLPSKGKMRQLEQALSARTLRKWSHSLQKRWIEVFIPRFSISASYNLETILPKMGIQNVFDKNADFSGIAKRDSLQVSKATHKAVLDVSEEGTEATAATTTKFIVRSKDGPSYFTVSFNRTFLMMITNKATDGILFLGKVENPTKS

SEQ ID NO: 23 - DNA, 1200 bp, Homo sapiens
atgagatcagctggagggagaggagagattaaagtgaggagagagctacaaccaagtaagcaagtgtcagggctcaccaaccatgcaaggacagggcaggagaagaggaacctgcaaaggctggttttggagaccccgagtcagaacatcttcttctcccctgtgagtgtctccacttccctggccatgctctcccttggggcccactcagtcaccaagacccagattctccagggcctgggcttcaacctcacacacacaccagagtctgccatccaccagggcttccagcacctggttcactcactgactgttcccagcaaagacctgaccttgaagatgggaagtgccctcttcgtcaagaaggagctgcagctgcaggcaaatttcttgggcaatgtcaagaggctgtatgaagcagaagtcttttctacagatttctccaacccctccattgcccaggcgaggatcaacagccatgtgaaaaagaagacccaagggaaggttgtagacataatccaaggccttgaccttctgacggccatggttctggtgaaccacattttctttaaagccaagtgggagaagccctttcaccctgaatatacaagaaagaacttcccattcctggtgggcgagcaggtcactgtgcatgtccccatgatgcaccagaaagagcagttcgcttttggggtggatacagagctgaactgctttgtgctgcagatggattacaagggagatgccgtggccttctttgtcctccctagcaagggcaagatgaggcaactggaacaggccttgtcagccagaacactgagaaagtggagccactcactccagaaaaggtggatagaggtgttcatccccagattttccatttctgcctcctacaatctggaaaccatcctcccgaagatgggcatccaaaatgtctttgacaaaaatgctgatttttctggaattgcaaagagagactccctgcaggtttctaaagcaacccacaaggctgtgctggatgtcagtgaagagggcactgaggccacagcagctaccaccaccaagttcatagtccgatcgaaggatggcccctcttacttcactgtctccttcaataggaccttcctgatgatgattacaaataaagccacagacggtattctctttctagggaaagtggaaaatcccactaaatcctag

SEQ ID NO: 24 - protein, 335 aa, Homo sapiens
MQGQGRRRGTCKDIFCSKMASYLYGVLFAVGLCAPIYCVSPANAPSAYPRPSSTKSTPASQVYSLNTDFAFRLYRRLVLETPSQNIFFSPARINSHVKKKTQGKVVDIIQGLDLLTAMVLVNHIFFKAKWEKPFHPEYTRKNFPFLVGEQVTVHVPMMHQKEQFAFGVDTELNCFVLQMDYKGDAVAFFVLPSKGKMRQLEQALSARTLRKWSHSLQKRWIEVFIPRFSISASYNLETILPKMGIQNVFDKNADFSGIAKRDSLQVSKATHKAVLDVSEEGTEATAATTTKFIVRSKDGPSYFTVSFNRTFLMMITNKATDGILFLGKVENPTKS

SEQ ID NO: 25 - DNA, 1008 bp, Homo sapiens
atgcaaggacagggcaggagaagaggaacctgcaaagacatattttgttccaaaatggcatcttacctttatggagtactctttgctgttggcctctgtgctccaatctactgtgtgtccccggccaatgcccccagtgcatacccccgcccttcctccacaaagagcacccctgcctcacaggtgtattccctcaacaccgactttgccttccgcctataccgcaggctggttttggagaccccgagtcagaacatcttcttctcccctgcgaggatcaacagccatgtgaaaaagaagacccaagggaaggttgtagacataatccaaggccttgaccttctgacggccatggttctggtgaaccacattttctttaaagccaagtgggagaagccctttcaccctgaatatacaagaaagaacttcccattcctggtgggcgagcaggtcactgtgcatgtccccatgatgcaccagaaagagcagttcgcttttggggtggatacagagctgaactgctttgtgctgcagatggattacaagggagatgccgtggccttctttgtcctccctagcaagggcaagatgaggcaactggaacaggccttgtcagccagaacactgagaaagtggagccactcactccagaaaaggtggatagaggtgttcatccccagattttccatttctgcctcctacaatctggaaaccatcctcccgaagatgggcatccaaaatgtctttgacaaaaatgctgatttttctggaattgcaaagagagactccctgcaggtttctaaagcaacccacaaggctgtgctggatgtcagtgaagagggcactgaggccacagcagctaccaccaccaagttcatagtccgatcgaaggatggcccctcttacttcactgtctccttcaataggaccttcctgatgatgattacaaataaagccacagacggtattctctttctagggaaagtggaaaatcccactaaatcctag

SEQ ID NO: 26 - protein, 435 aa, Homo sapiens
MQGQGRRRGTCKDIFCSKMASYLYGVLFAVGLCAPIYCVSPANAPSAYPRPSSTKSTPASQVYSLNTDFAFRLYRRLVLETPSQNIFFSPVSVSTSLAMLSLGAHSVTKTQILQGLGFNLTHTPESAIHQGFQHLVHSLTVPSKDLTLKMGSALFVKKELQLQANFLGNVKRLYEAEVFSTDFSNPSIAQARINSHVKKKTQGKVVDIIQGLDLLTAMVLVNHIFFKAKWEKPFHPEYTRKNFPFLVGEQVTVHVPMMHQKEQFAFGVDTELNCFVLQMDYKGDAVAFFVLPSKGKMRQLEQALSARTLRKWSHSLQKRWIEVFIPRFSISASYNLETILPKMGIQNVFDKNADFSGIAKRDSLQVSKATHKAVLDVSEEGTEATAATTTKFIVRSKDGPSYFTVSFNRTFLMMITNKATDGILFLGKVENPTKS

SEQ ID NO: 27 - DNA, 1308 bp, Homo sapiens
atgcaaggacagggcaggagaagaggaacctgcaaagacatattttgttccaaaatggcatcttacctttatggagtactctttgctgttggcctctgtgctccaatctactgtgtgtccccggccaatgcccccagtgcatacccccgcccttcctccacaaagagcacccctgcctcacaggtgtattccctcaacaccgactttgccttccgcctataccgcaggctggttttggagaccccgagtcagaacatcttcttctcccctgtgagtgtctccacttccctggccatgctctcccttggggcccactcagtcaccaagacccagattctccagggcctgggcttcaacctcacacacacaccagagtctgccatccaccagggcttccagcacctggttcactcactgactgttcccagcaaagacctgaccttgaagatgggaagtgccctcttcgtcaagaaggagctgcagctgcaggcaaatttcttgggcaatgtcaagaggctgtatgaagcagaagtcttttctacagatttctccaacccctccattgcccaggcgaggatcaacagccatgtgaaaaagaagacccaagggaaggttgtagacataatccaaggccttgaccttctgacggccatggttctggtgaaccacattttctttaaagccaagtgggagaagccctttcaccctgaatatacaagaaagaacttcccattcctggtgggcgagcaggtcactgtgcatgtccccatgatgcaccagaaagagcagttcgcttttggggtggatacagagctgaactgctttgtgctgcagatggattacaagggagatgccgtggccttctttgtcctccctagcaagggcaagatgaggcaactggaacaggccttgtcagccagaacactgagaaagtggagccactcactccagaaaaggtggatagaggtgttcatccccagattttccatttctgcctcctacaatctggaaaccatcctcccgaagatgggcatccaaaatgtctttgacaaaaatgctgatttttctggaattgcaaagagagactccctgcaggtttctaaagcaacccacaaggctgtgctggatgtcagtgaagagggcactgaggccacagcagctaccaccaccaagttcatagtccgatcgaaggatggcccctcttacttcactgtctccttcaataggaccttcctgatgatgattacaaataaagccacagacggtattctctttctagggaaagtggaaaatcccactaaatcctag

SEQ ID NO: 28 - protein, 444 aa, Homo sapiens
MKVVPSLLLSVLLAQVWLVPGLAPSPQSPETPAPQNQTSRVVQAPKEEEEDEQEASEEKASEEEKAWLMASRQQLAKETSNFGFSLLRKISMRHDGNMVFSPFGMSLAMTGLMLGATGPTETQIKRGLHLQALKPTKPGLLPSLFKGLRETLSRNLELGLTQGSFAFIHKDFDVKETFFNLSKRYFDTECVPMNFRNASQAKRLMNHYINKETRGKIPKLFDEINPETKLILVDYILFKGKWLTPFDPVFTEVDTFHLDKYKTIKVPMMYGAGKFASTFDKNFRCHVLKLPYQGNATMLVVLMEKMGDHLALEDYLTTDLVETWLRNMKTRNMEVFFPKFKLDQKYEMHELLRQMGIRRIFSPFADLSELSATGRNLQVSRVLQRTVIEVDERGTEAVAGILSEITAYSMPPVIKVDRPFHFMIYEETSGMLLFLGRVVNPTLL

SEQ ID NO: 29 - DNA, 1335 bp, Homo sapiens
atgaaggtggtgccaagtctcctgctctccgtcctcctggcacaggtgtggctggtacccggcttggcccccagtcctcagtcgccagagaccccagcccctcagaaccagaccagcagggtagtgcaggctcccaaggaggaagaggaagatgagcaggaggccagcgaggagaaggccagtgaggaagagaaagcctggctgatggccagcaggcagcagcttgccaaggagacttcaaacttcggattcagcctgctgcgaaagatctccatgaggcacgatggcaacatggtcttctctccatttggcatgtccttggccatgacaggcttgatgctgggggccacagggccgactgaaacccagatcaagagagggctccacttgcaggccctgaagcccaccaagcccgggctcctgccttccctctttaagggactcagagagaccctctcccgcaacctggaactgggcctcacacaggggagttttgccttcatccacaaggattttgatgtcaaagagactttcttcaatttatccaagaggtattttgatacagagtgcgtgcctatgaattttcgcaatgcctcacaggccaaaaggctcatgaatcattacattaacaaagagactcgggggaaaattcccaaactgtttgatgagattaatcctgaaaccaaattaattcttgtggattacatcttgttcaaagggaaatggttgaccccatttgaccctgtcttcaccgaagtcgacactttccacctggacaagtacaagaccattaaggtgcccatgatgtacggtgcaggcaagtttgcctccacctttgacaagaattttcgttgtcatgtcctcaaactgccctaccaaggaaatgccaccatgctggtggtcctcatggagaaaatgggtgaccacctcgcccttgaagactacctgaccacagacttggtggagacatggctcagaaacatgaaaaccagaaacatggaagttttctttccgaagttcaagctagatcagaagtatgagatgcatgagctgcttaggcagatgggaatcagaagaatcttctcaccctttgctgaccttagtgaactctcagctactggaagaaatctccaagtatccagggttttacaaagaacagtgattgaagttgatgaaaggggcactgaggcagtggcaggaatcttgtcagaaattactgcttattccatgcctcctgtcatcaaagtggaccggccatttcatttcatgatctatgaagaaacctctggaatgcttctgtttctgggcagggtggtgaatccgactctcctataa

SEQ ID NO: 30 - protein, 444 aa, Homo sapiens; identical to SEQ ID NO: 28

SEQ ID NO: 31 - DNA, 1335 bp, Homo sapiens; identical to SEQ ID NO: 29

SEQ ID NO: 32 - protein, 414 aa, Homo sapiens
MNPTLGLAIFLAVLLTVKGLLKPSFSPRNYKALSEVQGWKQRMAAKELARQNMDLGFKLLKKLAFYNPGRNIFLSPLSISTAFSMLCLGAQDSTLDEIKQGFNFRKMPEKDLHEGFHYIIHELTQKTQDLKLSIGNTLFIDQRLQPQRKFLEDAKNFYSAETILTNFQNLEMAQKQINDFISQKTHGKINNLIENIDPGTVMLLANYIFFRARWKHEFDPNVTKEEDFFLEKNSSVKVPMMFRSGIYQVGYDDKLSCTILEIPYQKNITAIFILPDEGKLKHLEKGLQVDTFSRWKTLLSRRVVDVSVPRLHMTGTFDLKKTLSYIGVSKIFEEHGDLTKIAPHRSLKVGEAVHKAELKMDERGTEGAAGTGAQTLPMETPLVVKIDKPYLLLIYSEKIPSVLFLGKIVNPIGK

SEQ ID NO: 33 - DNA, 1245 bp, Homo sapiens
atgaaccccacactaggcctggccatttttctggctgttctcctcacggtgaaaggtcttctaaagccgagcttctcaccaaggaattataaagctttgagcgaggtccaaggatggaagcaaaggatggcagccaaggagcttgcaaggcagaacatggacttaggctttaagctgctcaagaagctggccttttacaaccctggcaggaacatcttcctatcccccttgagcatctctacagctttctccatgctgtgcctgggtgcccaggacagcaccctggacgagatcaagcaggggttcaacttcagaaagatgccagaaaaagatcttcatgagggcttccattacatcatccacgagctgacccagaagacccaggacctcaaactgagcattgggaacacgctgttcattgaccagaggctgcagccacagcgtaagtttttggaagatgccaagaacttttacagtgccgaaaccatccttaccaactttcagaatttggaaatggctcagaagcagatcaatgactttatcagtcaaaaaacccatgggaaaattaacaacctgatcgagaatatagaccccggcactgtgatgcttcttgcaaattatattttctttcgagccaggtggaaacatgagtttgatccaaatgtaactaaagaggaagatttctttctggagaaaaacagttcagtcaaggtgcccatgatgttccgtagtggcatataccaagttggctatgacgataagctctcttgcaccatcctggaaataccctaccagaaaaatatcacagccatcttcatccttcctgatgagggcaagctgaagcacttggagaagggattgcaggtggacactttctccagatggaaaacattactgtcacgcagggtcgtagacgtgtctgtacccagactccacatgacgggcaccttcgacctgaagaagactctctcctacataggtgtctccaaaatctttgaggaacatggtgatctcaccaagatcgcccctcatcgcagcctgaaagtgggcgaggctgtgcacaaggctgagctgaagatggatgagaggggtacggaaggggccgctggcaccggagcacagactctgcccatggagacaccactcgtcgtcaagatagacaaaccctatctgctgctgatttacagcgagaaaataccttccgtgctcttcctgggaaagattgttaaccctattggaaaataa

SEQ ID NO: 34 - protein, 414 aa, Homo sapiens; identical to SEQ ID NO: 32

SEQ ID NO: 35 - DNA, 1245 bp, Homo sapiens; identical to SEQ ID NO: 33

SEQ ID NO: 36 - protein, 379 aa, Homo sapiens
MEQLSSANTRFALDLFLALSENNPAGNIFISPFSISSAMAMVFLGTRGNTAAQLSKTFHFNTVEEVHSRFQSLNADINKRGASYILKLANRLYGEKTYNFLPEFLVSTQKTYGADLASVDFQHASEDARKTINQWVKGQTEGKIPELLASGMVDNMTKLVLVNAIYFKGNWKDKFMKEATTNAPFRLNKKDRKTVKMMYQKKKFAYGYIEDLKCRVLELPYQGEELSMVILLPDDIEDESTGLKKIEEQLTLEKLHEWTKPENLDFIEVNVSLPRFKLEESYTLNSDLARLGVQDLFNSSKADLSGMSGARDIFISKIVHKSFVEVNEEGTEAAAATAGIATFCMLMPEENFTADHPFLFFIRHNSSGSILFLGRFSSP

SEQ ID NO: 37 - DNA, 1140 bp, Homo sapiens
atggagcagctgagctcagcaaacacccgcttcgccttggacctgttcctggcgttgagtgagaacaatccggctggaaacatcttcatctctcccttcagcatttcatctgctatggccatggtttttctggggaccagaggtaacacggcagcacagctgtccaagactttccatttcaacacggttgaagaggttcattcaagattccagagtctgaatgctgatatcaacaaacgtggagcgtcttatattctgaaacttgctaatagattatatggagagaaaacttacaatttccttcctgagttcttggtttcgactcagaaaacatatggtgctgacctggccagtgtggattttcagcatgcctctgaagatgcaaggaagaccataaaccagtgggtcaaaggacagacagaaggaaaaattccggaactgttggcttcgggcatggttgataacatgaccaaacttgtgctagtaaatgccatctatttcaagggaaactggaaggataaattcatgaaagaagccacgacgaatgcaccattcagattgaataagaaagacagaaaaactgtgaaaatgatgtatcagaagaaaaaatttgcatatggctacatcgaggaccttaagtgccgtgtgctggaactgccttaccaaggcgaggagctcagcatggtcatcctgctgccggatgacattgaggacgagtccacgggcctgaagaagattgaggaacagttgactttggaaaagttgcatgagtggactaaacctgagaatctcgatttcattgaagttaatgtcagcttgcccaggttcaaactggaagagagttacactctcaactccgacctcgcccgcctaggtgtgcaggatctctttaacagtagcaaggctgatctgtctggcatgtcaggagccagagatatttttatatcaaaaattgtccacaagtcatttgtggaagtgaatgaagagggaacagaggcggcagctgccacagcaggcatcgcaactttctgcatgttgatgcccgaagaaaatttcactgccgaccatccattccttttctttattcggcataattcctcaggtagcatcctattcttggggagattttcttccccttag

SEQ ID NO: 38 - protein, 376 aa, Homo sapiens
MDVLAEANGTFALNLLKTLGKDNSKNVFFSPMSMSCALAMVYMGAKGNTAAQMAQILSFNKSGGGGDIHQGFQSLLTEVNKTGTQYLLRMANRLFGEKSCDFLSSFRDSCQKFYQAEMEELDFISAVEKSRKHINTWVAEKTEGKIAELLSPGSVDPLTRLVLVNAVYFRGNWDEQFDKENTEERLFKVSKNEEKPVQMMFKQSTFKKTYIGEIFTQILVLPYVGKELNMIIMLPDETTDLRTVEKELTYEKFVEWTRLDMMDEEEVEVSLPRFKLEESYDMESVLRNLGMTDAFELGKADFSGMSQTDLSLSKVVHKSFVEVNEEGTEAAAATAAIMMMRCARFVPRFCADHPFLFFIQHSKTNGILFCGRFSSP

SEQ ID NO: 39 - DNA, 1131 bp, Homo sapiens
atggatgttctcgcagaagcaaatggcacctttgccttaaaccttttgaaaacgctgggtaaagacaactcgaagaatgtgtttttctcacccatgagcatgtcctgtgccctggccatggtctacatgggggcaaagggaaacaccgctgcacagatggcccagatactttctttcaataaaagtggcggtggtggagacatccaccagggcttccagtctcttctcaccgaagtgaacaagactggcacgcagtacttgcttaggatggccaacaggctctttggggaaaagtcttgtgatttcctctcatcttttagagattcctgccaaaaattctaccaagcagagatggaggagcttgactttatcagcgccgtagagaagtccagaaaacacataaacacctgggtagctgaaaagacagaaggtaaaattgcggagttgctctctccgggctcagtggatccattgacaaggctggttctggtgaatgctgtctatttcagaggaaactgggatgaacagtttgacaaggagaacaccgaggagagactgtttaaagtcagcaagaatgaggagaaacctgtgcaaatgatgtttaagcaatctacttttaagaagacctatataggagaaatatttacccaaatcttggtgcttccatatgttggcaaggaactgaatatgatcatcatgcttccggacgagaccactgacttgagaacggtggagaaagaactcacttacgagaagttcgtagaatggacgaggctggacatgatggatgaagaggaggtggaagtgtccctcccgcggtttaaactagaggaaagctacgacatggagagtgtcctgcgcaacctgggcatgactgatgccttcgagctgggcaaggcagacttctctggaatgtcccagacagacctgtctctgtccaaggtcgtgcacaagtcttttgtggaggtcaatgaggaaggcacggaggctgcagccgccacagctgccatcatgatgatgcggtgtgccagattcgtcccccgcttctgcgccgaccaccccttccttttcttcatccagcacagcaagaccaacgggattctcttctgcggccgcttttcctctccgtga

SEQ ID NO: 40 - protein, 380 aa, Homo sapiens
MSAIMDVLAEANGTFALNLLKTLGKDNSKNVFFSPMSMSCALAMVYMGAKGNTAAQMAQILSFNKSGGGGDIHQGFQSLLTEVNKTGTQYLLRMANRLFGEKSCDFLSSFRDSCQKFYQAEMEELDFISAVEKSRKHINTWVAEKTEGKIAELLSPGSVDPLTRLVLVNAVYFRGNWDEQFDKENTEERLFKVSKNEEKPVQMMFKQSTFKKTYIGEIFTQILVLPYVGKELNMIIMLPDETTDLRTVEKELTYEKFVEWTRLDMMDEEEVEVSLPRFKLEESYDMESVLRNLGMTDAFELGKADFSGMSQTDLSLSKVVHKSFVEVNEEGTEAAAATAAIMMMRCARFVPRFCADHPFLFFIQHSKTNGILFCGRFSSP

SEQ ID NO: 41 - DNA, 1143 bp, Homo sapiens
atgtctgccatcatggatgttctcgcagaagcaaatggcacctttgccttaaaccttttgaaaacgctgggtaaagacaactcgaagaatgtgtttttctcacccatgagcatgtcctgtgccctggccatggtctacatgggggcaaagggaaacaccgctgcacagatggcccagatactttctttcaataaaagtggcggtggtggagacatccaccagggcttccagtctcttctcaccgaagtgaacaagactggcacgcagtacttgcttaggatggccaacaggctctttggggaaaagtcttgtgatttcctctcatcttttagagattcctgccaaaaattctaccaagcagagatggaggagcttgactttatcagcgccgtagagaagtccagaaaacacataaacacctgggtagctgaaaagacagaaggtaaaattgcggagttgctctctccgggctcagtggatccattgacaaggctggttctggtgaatgctgtctatttcagaggaaactgggatgaacagtttgacaaggagaacaccgaggagagactgtttaaagtcagcaagaatgaggagaaacctgtgcaaatgatgtttaagcaatctacttttaagaagacctatataggagaaatatttacccaaatcttggtgcttccatatgttggcaaggaactgaatatgatcatcatgcttccggacgagaccactgacttgagaacggtggagaaagaactcacttacgagaagttcgtagaatggacgaggctggacatgatggatgaagaggaggtggaagtgtccctcccgcggtttaaactagaggaaagctacgacatggagagtgtcctgcgcaacctgggcatgactgatgccttcgagctgggcaaggcagacttctctggaatgtcccagacagacctgtctctgtccaaggtcgtgcacaagtcttttgtggaggtcaatgaggaaggcacggaggctgcagccgccacagctgccatcatgatgatgcggtgtgccagattcgtcccccgcttctgcgccgaccaccccttccttttcttcatccagcacagcaagaccaacgggattctcttctgcggccgcttttcctctccgtga

SEQ ID NO: 42 - protein, 390 aa, Homo sapiens
MGAAQSLPGHRSAIMDVLAEANGTFALNLLKTLGKDNSKNVFFSPMSMSCALAMVYMGAKGNTAAQMAQILSFNKSGGGGDIHQGFQSLLTEVNKTGTQYLLRMANRLFGEKSCDFLSSFRDSCQKFYQAEMEELDFISAVEKSRKHINTWVAEKTEGKIAELLSPGSVDPLTRLVLVNAVYFRGNWDEQFDKENTEERLFKVSKNEEKPVQMMFKQSTFKKTYIGEIFTQILVLPYVGKELNMIIMLPDETTDLRTVEKELTYEKFVEWTRLDMMDEEEVEVSLPRFKLEESYDMESVLRNLGMTDAFELGKADFSGMSQTDLSLSKVVHKSFVEVNEEGTEAAAATAAIMMMRCARFVPRFCADHPFLFFIQHSKTNGILFCGRFSSP

SEQ ID NO: 43 - DNA, 1173 bp, Homo sapiens
atgggggcggcgcagagcctcccgggccacaggtctgccatcatggatgttctcgcagaagcaaatggcacctttgccttaaaccttttgaaaacgctgggtaaagacaactcgaagaatgtgtttttctcacccatgagcatgtcctgtgccctggccatggtctacatgggggcaaagggaaacaccgctgcacagatggcccagatactttctttcaataaaagtggcggtggtggagacatccaccagggcttccagtctcttctcaccgaagtgaacaagactggcacgcagtacttgcttaggatggccaacaggctctttggggaaaagtcttgtgatttcctctcatcttttagagattcctgccaaaaattctaccaagcagagatggaggagcttgactttatcagcgccgtagagaagtccagaaaacacataaacacctgggtagctgaaaagacagaaggtaaaattgcggagttgctctctccgggctcagtggatccattgacaaggctggttctggtgaatgctgtctatttcagaggaaactgggatgaacagtttgacaaggagaacaccgaggagagactgtttaaagtcagcaagaatgaggagaaacctgtgcaaatgatgtttaagcaatctacttttaagaagacctatataggagaaatatttacccaaatcttggtgcttccatatgttggcaaggaactgaatatgatcatcatgcttccggacgagaccactgacttgagaacggtggagaaagaactcacttacgagaagttcgtagaatggacgaggctggacatgatggatgaagaggaggtggaagtgtccctcccgcggtttaaactagaggaaagctacgacatggagagtgtcctgcgcaacctgggcatgactgatgccttcgagctgggcaaggcagacttctctggaatgtcccagacagacctgtctctgtccaaggtcgtgcacaagtcttttgtggaggtcaatgaggaaggcacggaggctgcagccgccacagctgccatcatgatgatgcggtgtgccagattcgtcccccgcttctgcgccgaccaccccttccttttcttcatccagcacagcaagaccaacgggattctcttctgcggccgcttttcctctccgtga

SEQ ID NO: 44 - protein, 395 aa, Homo sapiens
MSSRQRGNFNYKLAFKSAIMDVLAEANGTFALNLLKTLGKDNSKNVFFSPMSMSCALAMVYMGAKGNTAAQMAQILSFNKSGGGGDIHQGFQSLLTEVNKTGTQYLLRMANRLFGEKSCDFLSSFRDSCQKFYQAEMEELDFISAVEKSRKHINTWVAEKTEGKIAELLSPGSVDPLTRLVLVNAVYFRGNWDEQFDKENTEERLFKVSKNEEKPVQMMFKQSTFKKTYIGEIFTQILVLPYVGKELNMIIMLPDETTDLRTVEKELTYEKFVEWTRLDMMDEEEVEVSLPRFKLEESYDMESVLRNLGMTDAFELGKADFSGMSQTDLSLSKVVHKSFVEVNEEGTEAAAATAAIMMMRCARFVPRFCADHPFLFFIQHSKTNGILFCGRFSSP

SEQ ID NO: 45 - DNA, 1188 bp, Homo sapiens
atgtcttcaaggcaaagaggaaactttaactacaaattggcatttaagtctgccatcatggatgttctcgcagaagcaaatggcacctttgccttaaaccttttgaaaacgctgggtaaagacaactcgaagaatgtgtttttctcacccatgagcatgtcctgtgccctggccatggtctacatgggggcaaagggaaacaccgctgcacagatggcccagatactttctttcaataaaagtggcggtggtggagacatccaccagggcttccagtctcttctcaccgaagtgaacaagactggcacgcagtacttgcttaggatggccaacaggctctttggggaaaagtcttgtgatttcctctcatcttttagagattcctgccaaaaattctaccaagcagagatggaggagcttgactttatcagcgccgtagagaagtccagaaaacacataaacacctgggtagctgaaaagacagaaggtaaaattgcggagttgctctctccgggctcagtggatccattgacaaggctggttctggtgaatgctgtctatttcagaggaaactgggatgaacagtttgacaaggagaacaccgaggagagactgtttaaagtcagcaagaatgaggagaaacctgtgcaaatgatgtttaagcaatctacttttaagaagacctatataggagaaatatttacccaaatcttggtgcttccatatgttggcaaggaactgaatatgatcatcatgcttccggacgagaccactgacttgagaacggtggagaaagaactcacttacgagaagttcgtagaatggacgaggctggacatgatggatgaagaggaggtggaagtgtccctcccgcggtttaaactagaggaaagctacgacatggagagtgtcctgcgcaacctgggcatgactgatgccttcgagctgggcaaggcagacttctctggaatgtcccagacagacctgtctctgtccaaggtcgtgcacaagtcttttgtggaggtcaatgaggaaggcacggaggctgcagccgccacagctgccatcatgatgatgcggtgtgccagattcgtcccccgcttctgcgccgaccaccccttccttttcttcatccagcacagcaagaccaacgggattctcttctgcggccgcttttcctctccgtga

SEQ ID NO: 46 - protein, 376 aa, Homo sapiens; identical to SEQ ID NO: 38

SEQ ID NO: 47 - DNA, 1131 bp, Homo sapiens; identical to SEQ ID NO: 39

SEQ ID NO: 48 - protein, 376 aa, Homo sapiens; identical to SEQ ID NO: 38

SEQ ID NO: 49 - DNA, 1131 bp, Homo sapiens; identical to SEQ ID NO: 39

SEQ ID NO: 50 - protein, 376 aa, Homo sapiens; identical to SEQ ID NO: 38

SEQ ID NO: 51 - DNA, 1131 bp, Homo sapiens; identical to SEQ ID NO: 39

SEQ ID NO: 52 - protein, 376 aa, Homo sapiens; identical to SEQ ID NO: 38

SEQ ID NO: 53 - DNA, 1131 bp, Homo sapiens; identical to SEQ ID NO: 39

SEQ ID NO: 54 - protein, 376 aa, Homo sapiens; identical to SEQ ID NO: 7

SEQ ID NO: 55 - DNA, 1131 bp, Homo sapiens
atggaaactctttctaatgcaagtggtacttttgccatacgccttttaaagatactgtgtcaagataacccttcgcacaacgtgttctgttctcctgtgagcatctcctctgccctggccatggttctcctaggggcaaagggaaacaccgcaacccagatggcccaggcactgtctttaaacacagaggaagacattcatcgggctttccagtcgcttctcactgaagtgaacaaggctggcacacagtacctgctgagaacggccaacaggctctttggagagaaaacttgtcagttcctctcaacgtttaaggaatcctgtcttcaattctaccatgctgagctgaaggagctttcctttatcagagctgcagaagagtccaggaaacacatcaacacctgggtctcaaaaaagaccgaaggtaaaattgaagagttgttgccgggtagctcaattgatgcagaaaccaggctggttcttgtcaatgccatctacttcaaaggaaagtggaatgaaccgtttgacgaaacatacacaagggaaatgccctttaaaataaaccaggaggagcaaaggccagtgcagatgatgtatcaggaggccacgtttaagctcgcccacgtgggcgaggtgcgcgcgcagctgctggagctgccctacgccaggaaggagctgagcctgctggtgctgctgcctgacgacggcgtggagctcagcacggtggaaaaaagtctcacttttgagaaactcacagcctggaccaagccagactgtatgaagagtactgaggttgaagttctccttccaaaatttaaactacaagaggattatgacatggaatctgtgcttcggcatttgggaattgttgatgccttccaacagggcaaggctgacttgtcggcaatgtcagcggagagagacctgtgtctgtccaagttcgtgcacaagagttttgtggaggtgaatgaagaaggcaccgaggcagcggcagcgtcgagctgctttgtagttgcagagtgctgcatggaatctggccccaggttctgtgctgaccaccctttccttttcttcatcaggcacaacagagccaacagcattctgttctgtggcaggttctcatcgccataa

SEQ ID NO: 56 - protein, 500 aa, Homo sapiens
MASRLTLLTLLLLLLAGDRASSNPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

SEQ ID NO: 57 - DNA, 1503 bp, Homo sapiens
atggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctga

SEQ ID NO: 58 - protein, 500 aa, Homo sapiens; identical to SEQ ID NO: 56

SEQ ID NO: 59 - DNA, 1503 bp, Homo sapiens; identical to SEQ ID NO: 57

SEQ ID NO: 60 - protein, 410 aa, Homo sapiens
MAFLGLFSLLVLQSMATGATFPEEAIADLSVNMYNRLRATGEDENILFSPLSIALAMGMMELGAQGSTQKEIRHSMGYDSLKNGEEFSFLKEFSNMVTAKESQYVMKIANSLFVQNGFHVNEEFLQMMKKYFNAAVNHVDFSQNVAVANYINKWVENNTNNLVKDLVSPRDFDAATYLALINAVYFKGNWKSQFRPENTRTFSFTKDDESEVQIPMMYQQGEFYYGEFSDGSNEAGGIYQVLEIPYEGDEISMMLVLSRQEVPLATLEPLVKAQLVEEWANSVKKQKVEVYLPRFTVEQEIDLKDVLKALGITEIFIKDANLTGLSDNKEIFLSKAIHKSFLEVNEEGSEAAAVSGMIAISRMAVLYPQVIVDHPFFFLIRNRRTGTILFMGRVMHPETMNTSGHDFEEL

SEQ ID NO: 61 - DNA, 1233 bp, Homo sapiens
atggctttccttggactcttctctttgctggttctgcaaagtatggctacaggggccactttccctgaggaagccattgctgacttgtcagtgaatatgtataatcgtcttagagccactggtgaagatgaaaatattctcttctctccattgagtattgctcttgcaatgggaatgatggaacttggggcccaaggatctacccagaaagaaatccgccactcaatgggatatgacagcctaaaaaatggtgaagaattttctttcttgaaggagttttcaaacatggtaactgctaaagagagccaatatgtgatgaaaattgccaattccttgtttgtgcaaaatggatttcatgtcaatgaggagtttttgcaaatgatgaaaaaatattttaatgcagcagtaaatcatgtggacttcagtcaaaatgtagccgtggccaactacatcaataagtgggtggagaataacacaaacaatctggtgaaagatttggtatccccaagggattttgatgctgccacttatctggccctcattaatgctgtctatttcaaggggaactggaagtcgcagtttaggcctgaaaatactagaaccttttctttcactaaagatgatgaaagtgaagtccaaattccaatgatgtatcagcaaggagaattttattatggggaatttagtgatggctccaatgaagctggtggtatctaccaagtcctagaaataccatatgaaggagatgaaataagcatgatgctggtgctgtccagacaggaagttcctcttgctactctggagccattagtcaaagcacagctggttgaagaatgggcaaactctgtgaagaagcaaaaagtagaagtatacctgcccaggttcacagtggaacaggaaattgatttaaaagatgttttgaaggctcttggaataactgaaattttcatcaaagatgcaaatttgacaggcctctctgataataaggagatttttctttccaaagcaattcacaagtccttcctagaggttaatgaagaaggctcagaagctgctgctgtctcaggaatgattgcaattagtaggatggctgtgctgtatcctcaagttattgtcgaccatccatttttctttcttatcagaaacaggagaactggtacaattctattcatgggacgagtcatgcatcctgaaacaatgaacacaagtggacatgatttcgaagaactttaa

SEQ ID NO: 62 - protein, 410 aa, Homo sapiens; identical to SEQ ID NO: 60

SEQ ID NO: 63 - DNA, 1233 bp, Homo sapiens; identical to SEQ ID NO: 61

(19) United States
(12) Patent Application Publication
(10) Pub. No.: US 2018/0050082 A1
(43) Pub. Date: Feb. 22, 2018

(54) SERPINS FOR THE TREATMENT OF NEUROINFLAMMATORY DISEASES

(71) Applicant: Anvil Biosciences, LLC, Redwood City, CA (US)

(72) Inventors: Jack Tzu-Chiao Lin, San Bruno, CA (US); Emily A. Stein, San Leandro, CA (US)

(73) Assignee: Anvil Biosciences, LLC, Redwood City, CA (US)

(21) Appl. No.: 15/556,608

(22) PCT Filed: Mar. 9, 2016
(86) PCT No.: PCT/US16/21412

§ 371 Date: Sep. 7, 2017

Related U.S. Application Data
(60) Provisional application No. 62/131,073, filed on Mar. 10, 2015.

(51) Int. Cl. A61K 38/16; A61K 31/573; A61K 39/395; A61K 38/54

(52) U.S. Cl. CPC A61K 38/16; A61K 38/54; A61K 39/395; A61K 31/573

(57) ABSTRACT

The present invention relates to the use of serpins, including A1AT, its derivatives and analogs thereof, in the prevention or treatment of neuroinflammatory diseases. In particular embodiments, the invention relates to the combination of A1AT and another anti-inflammatory therapeutic compound. The present invention further relates to methods for administering said A1AT combination.

### CROSS REFERENCE TO RELATED APPLICATIONS

This application claims the benefit of U.S. Provisional Application No. 62/131,073, filed on Mar. 10, 2015, incorporated herein by reference in its entirety.

### SEQUENCE LISTING

The instant application contains a Sequence Listing which has been filed electronically in ASCII format and is hereby incorporated by reference in its entirety. Said ASCII copy, created on Mar. 3, 2016, is named ANV002W_SL.txt and is 161,319 bytes in size.

### FIELD OF THE INVENTION

The present invention relates to the use of serpins, including alpha-1 antitrypsin (A1AT or AAT), its derivatives and analogs thereof, in the prevention or treatment of neuroinflammatory diseases. In particular embodiments, the invention relates to the combination of A1AT and another anti-inflammatory therapeutic compound. The present invention further relates to methods of administering said A1AT combination.

### BACKGROUND OF THE INVENTION

Alpha-1 antitrypsin (A1AT) is a member of the serpin superfamily of protease inhibitors. Normally found in serum, A1AT inhibits a wide variety of proteases and has been shown to protect tissues from the enzymes secreted by activated immune cells. Alpha-1 antitrypsin (AAT) inhibits IL-8 production as well as IL-8 binding to its receptors. A1AT also inactivates elastase to decrease extracellular matrix degradation of the blood-brain barrier. A1AT inhibits macrophage production of pro-inflammatory cytokines that are upregulated in neuromyelitis optica (NMO), a neurodegenerative disease. A1AT induces Treg cells, tolerogenic dendritic cells, and anti-inflammatory cytokines. Circulating levels of A1AT vary with a normal reference range in the blood of 1.5-3.5 g/L in humans. Since it is a negative feedback molecule that downregulates immune system activity, it is used as a marker of inflammation.

Serpins inactivate enzymes such as neutrophil elastase by covalently binding to the protease in a manner that inhibits enzyme activity. During infection or acute phase response, degranulation rates of immune cells increase markedly, thus high levels of serpins are required for enzyme neutralization and to limit damage to tissue. In circumstances where there are insufficient serpin concentrations in the tissue, fibrosis or scaring of the tissue can arise.

Several genetic mutations have been identified in humans that correspond to the incorrect folding of the beta sheets and alpha helices of A1AT and render the protein non-functional. This can cause A1AT deficiency and results in hepatic cirrhosis and fibrosis throughout the body.

Blood-derived A1AT has been used clinically to rescue patients deficient in A1AT. It has also been used recently to neutralize the effects of neutrophil elastase in patients with emphysema and chronic obstructive pulmonary disease.

Work by Subramanian et al. has demonstrated that sustained expression of human A1AT in a transgenic C57BL/6 mouse background were resistant to MOG-35-55 peptide-induced experimental autoimmune encephalomyelitis. Furthermore, hA1AT expression was also characteristic of elevated CD4+FoxP3+T_{reg} cell counts and diminished pro-inflammatory cytokine expression IL17, IL1b and IL6 and reduced CCR6 chemokine levels. (Metab. Brain Dis. 26(2):107-13 (2011), incorporated by reference herein in its entirety.)

NMO is a rare disorder that resembles multiple sclerosis in several ways but requires a different treatment regimen for disease maintenance. Symptoms of NMO include loss of vision and spinal cord function. Optical neuritis may manifest in patients as visual impairment with decreased visual acuity. Spinal cord demyelination may manifest in patients as muscle weakness, reduced sensory proprioception or loss of bladder and bowel control. During severe flares, patients may experience acute paraparesis or quadriparesis.

Recently, neutrophil elastase involvement, driven by granulocte-mediated inflammation, has been demonstrated in a mouse model of NMO. (See Mult. Scler. 18:398-408 (2012), incorporated by reference herein in its entirety.) At least two different causes are known for NMO including the presence of autoantibodies against aquaporin 4 and aberrant astrocyte activity. An increasing body of evidence also supports the contribution of Th17 cells and a role of granulocytic release at the site of inflammation.

Diagnosis of NMO is currently accepted as requiring two absolute criteria plus at least two supportive criteria. The absolute criteria are optic neuritis and acute myelitis whilst the supportive criteria are brain MRI not consistent with multiple sclerosis at disease onset, spinal cord MRI with contiguous T2-weighted signal abnormality extending over three or more vertebral segments and NMO-IgG seropositivity against aquaporin 4 antigen.

Multiple sclerosis (MS) is a heterogeneous condition consisting of recurrent and simultaneous inflammatory lesions of the spinal cord and brain causing demyelination of nerves in the central nervous system. Symptoms range widely and may be physical (motor function loss, sensory function loss, or pain) or psychological (mood alteration, depression). Several forms of MS exist including remitting and progressive forms. Specifically, forms include relapse-remitting, secondary progressive, primary progressive and progressive relapsing.

Diagnosis of MS is typically based on presenting symptoms with the most common diagnostic tools being neuroimaging, analysis of cerebrospinal fluid (CSF) and evoked potentials. In neuroimaging of patients suspected of having MS, MRI may be used to identify demyelinated areas of the brain and spinal cord. Gadolinium can be administered intravenously to highlight active inflammatory lesions. CSF can be obtained by lumbar puncture and can be used to measure CNS levels of inflammation, mainly oligoclonal bands of IgG by electrophoresis, and cytokines IL17, IL8, IL1b and T cells. Brain responses can be examined using visual and sensory evoked potentials.

Amyotrophic lateral sclerosis (ALS) is a heterogeneous condition involving neuronal cell death. Symptoms of ALS include muscle stiffness and/or muscle twitching with gradual progression to muscle weakness and wasting. Patients with ALS have difficulty speaking, swallowing and eventually breathing. There are currently no definitive diagnostic tests for ALS and the majority of clinical testing is used to rule out other diseases. Often electromyography and nerve conduction velocity testing are used. Magnetic Resonance Imaging is often indeterminate with ALS patients. The disease presents symptoms that include muscle stiffness and twitching in a single limb involving upper or lower motor neurons.

Therapeutic inhibition of granulocyte proteases by sivelestat has been proposed (WO2011100567, incorporated by reference herein in its entirety.). Due to Sivelestat's low potency and therapeutic efficacy, however, more improved treatment regimens are needed for neuroinflammatory diseases associated with significant granulocyte involvement.

### SUMMARY OF THE INVENTION

The present invention relates to the use of serpins, including A1AT, its derivatives and analogs thereof, in the prevention or treatment of neuroinflammatory diseases. In particular embodiments, the invention relates to the combination of A1AT and methylprednisolone. The present invention further relates to methods administering said A1AT combination.

Thus, the invention provides a method of treating an inflammatory condition in a subject, comprising administering a serpin protein and administering methylprednisolone. In a preferred embodiment, the serpin protein and methylprednisolone are administered simultaneously.

In other embodiments, the inflammatory condition is neuromyelitis optica (NMO), multiple sclerosis (MS) or amyotrophic lateral sclerosis (ALS).

In some embodiments, the serpin protein has at least a 90% sequence identity to SEQ ID NO:1 and has alpha-1 antitrypsin (A1AT) activity. In a preferred embodiment, the serpin protein has the sequence of SEQ ID NO:1. In another embodiment, the serpin protein is encoded by a nucleic acid that has at least a 90% sequence identity to SEQ ID NO:2 and wherein said serpin protein has alpha-1 antitrypsin (A1AT) activity. In a preferred embodiment, the serpin protein is encoded by a nucleic acid that has the sequence of SEQ ID NO:2.

The invention provides a method of treating an inflammatory condition in a subject, comprising administering a nucleic acid that encodes a serpin protein and administering methylprednisolone. In some embodiments, the inflammatory condition is neuromyelitis optica (NMO), multiple sclerosis (MS), or amyotrophic lateral sclerosis (ALS).

In some embodiments, the nucleic acid encodes a protein having at least a 90% sequence identity to SEQ ID NO:1 and has alpha-1 antitrypsin (A1AT) activity. In a preferred embodiment, the nucleic acid encodes a protein having the sequence of SEQ ID NO:1. In other embodiments, the nucleic acid has at least a 90% sequence identity to SEQ ID NO:2 and the serpin protein has alpha-1 antitrypsin (A1AT) activity. In a preferred embodiment, the nucleic acid has the sequence of SEQ ID NO:2. In other embodiments, the nucleic acid is administered by a route selected from the group consisting of transfected autologous patient cells, viral vectors, and naked nucleic acid preparations.

The invention provides a method of treating an inflammatory condition in a subject, comprising increasing the expression of an endogenous serpin protein and administering methylprednisolone. In other embodiments, the inflammatory condition is neuromyelitis optica (NMO), multiple sclerosis (MS), or amyotrophic lateral sclerosis (ALS).

In some embodiments, the endogenous serpin protein has at least a 90% sequence identity to SEQ ID NO:1 and has alpha-1 antitrypsin (A1AT) activity. In a preferred embodiment, the endogenous serpin protein has the sequence of SEQ ID NO:1. In other embodiments, the endogenous serpin protein is encoded by a nucleic acid that has at least a 90% sequence identity to SEQ ID NO:2 and wherein the serpin protein has alpha-1 antitrypsin (A1AT) activity. In a preferred embodiment, the endogenous serpin protein is encoded by a nucleic acid that has the sequence of SEQ ID NO:2. In other embodiments, the increase in serpin expression is accomplished using a technology selected from the group consisting of zinc finger nucleases (ZFNs), transcription activator-like effector nucleases (TALENs), and clustered regularly interspaced short palindromic repeats (CRISPR)/Cas9.

The invention provides a pharmaceutical composition, comprising a serpin protein and methylprednisolone. In some embodiments, the serpin protein within the pharmaceutical composition has at least a 90% sequence identity to SEQ ID NO:1 and has alpha-1 antitrypsin (A1AT) activity. In a preferred embodiment, the serpin protein has the sequence of SEQ ID NO:1. In other embodiments, the serpin protein is encoded by a nucleic acid that has at least a 90% sequence identity to SEQ ID NO:2 and wherein the serpin protein has alpha-1 antitrypsin (A1AT) activity. In a preferred embodiment, the serpin protein within the pharmaceutical composition is encoded by a nucleic acid that has the sequence of SEQ ID NO:2.

### BRIEF DESCRIPTION OF THE DRAWINGS

FIG. 1 shows A1AT treatment of Neuromyelitis Optica (NMO) induced by the adoptive transfer of Th17 Experimental Autoimmune Encephalomyelitis (EAE) cells to C57BL/6 mice.

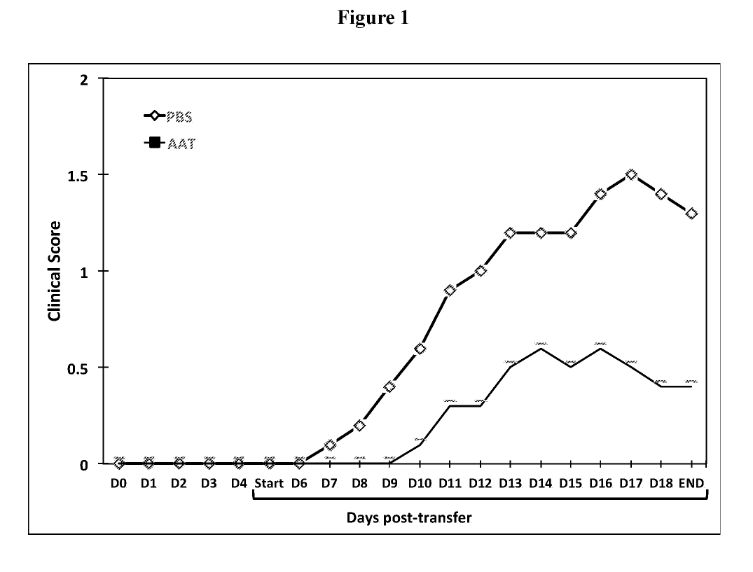

FIG. 2 shows that A1AT treatment reduced the NMO disease score compared to PBS (control) and was superior to Sivelestat at all days of overt signs of disease as well as delay onset of disease.

FIG. 3 shows mean NMO disease scores are reduced by A1AT+Mpred treatment when compared to no treatment (PBS) or either treatment alone. The data represent 10 recipient mice per cohort in a blinded study and is expressed as the mean disease score+/−SEM.

FIG. 4 shows that A1AT+Mpred resulted in less body weight loss than either treatment alone. Ten recipient mice per cohort were used in this blinded study. The graph shows mean body weight as a percentage of the value measured at day 0+/−SEM.

FIG. 5A shows that A1AT alone and A1AT+Mpred decreased the inflammatory cytokine IL-17A when compared to PBS and Mpred alone.

FIG. 5B shows that A1AT alone and A1AT+Mpred decreased the inflammatory cytokine IFN-γ when compared to Mpred alone.

FIG. 5C shows that all treatments increased IL-6 compared to PBS. A1AT+Mpred, however, showed the most significant reduction.

FIG. 5D shows that IL-2 levels were higher without antigenic restimulation (0 MOG peptide) when treated with A1AT alone or A1AT+Mpred when compared to Mpred alone.

### DETAILED DESCRIPTION OF THE INVENTION

The invention provides for the treatment or prevention of neuroinflammatory or neurodegenerative diseases with the combination of a serpin and another anti-inflammatory therapeutic compound. In particular embodiments, the neuroinflammatory condition is neuromyelitis optica (NMO), multiple sclerosis (MS), or amyotrophic lateral sclerosis (ALS). In other embodiments, the disease is associated with abnormal levels of Th17, CCR6 or IL8. In yet other embodiments, the Serpin is alpha-1 antitrypsin (A1AT). In other embodiments, the anti-inflammatory compound is methylprednisolone. In yet other embodiments of the invention, the therapeutic combinations disclosed herein are administered following diagnosis of a neuroinflammatory or neurodegenerative disease using a diagnostic test that measures circulating IL17, CCR6, IL8, anti-aquaporin 4 antibodies, Kir4 antibodies, neutrophil elastase or A1AT levels.

In describing and claiming one or more embodiments of the present invention, the following terminology will be used in accordance with the definitions described below:

The singular form “a”, “an”, and “the” includes plural references unless indicated otherwise.

The term “absorption” is the movement of a drug into the bloodstream. A drug needs to be introduced via some route of administration. For example, drugs of the invention may be delivered by oral, buccal, topical, dermal, inhalation, nasal, subcutaneous, intramuscular, or intravenous route or by any other route known in the pharmaceutical arts. Exemplary dosage forms include a solution, emulsion, inhalable powder, suspension, tablet, patch, capsule or other liquid.

“Amyotrophic lateral sclerosis” (ALS) as used herein includes, without limitation, a heterogeneous condition involving neuronal cell death. Symptoms of ALS include muscle stiffness and/or muscle twitching with gradual progression to muscle weakness and wasting. Patients with ALS have difficulty speaking, swallowing and eventually breathing.

A “clinician” or “medical researcher” or “veterinarian” as used herein, can include, without limitation, doctors, nurses, physician assistants, lab technicians, research scientists, clerical workers employed by the same, or any person involved in determining, diagnosing, aiding in the diagnosis or influencing the course of treatment for the individual.

An “effective amount” refers to an amount of therapeutic compound that is effective, at dosages and for periods of time necessary, to achieve the desired therapeutic or prophylactic result. A “therapeutically effective amount” of a therapeutic compound may vary according to factors such as the disease state, age, sex, and weight of the individual. A therapeutically effective amount may be measured, for example, by improved survival rate, more rapid recovery, or amelioration, improvement or elimination of symptoms, or other acceptable biomarkers or surrogate markers. A “therapeutically effective amount” is also one in which any toxic or detrimental effects of the therapeutic compound are outweighed by the therapeutically beneficial effects. A “prophylactically effective amount” refers to an amount of therapeutic compound that is effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount.

“Homologs” are bioactive molecules that are similar to a reference molecule at the nucleotide sequence, peptide sequence, functional, or structural level. Homologs may include sequence derivatives that share a certain percent identity with the reference sequence. Thus, in one embodiment, homologous or derivative sequences share at least a 70 percent sequence identity. In a preferred embodiment, homologous or derivative sequences share at least an 80 or 85 percent sequence identity. In a more preferred embodiment, homologous or derivative sequences share at least an 86, 87, 88, 89, 90, 91, 92, 93, 94, 95, 96, 97, 98, or 99 percent sequence identity. Homologous or derivative nucleic acid sequences may also be defined by their ability to remain bound to a reference nucleic acid sequence under high stringency hybridization conditions. Homologs having a structural or functional similarity to a reference molecule may be chemical derivatives of the reference molecule. Methods of detecting, generating, and screening for structural and functional homologs as well as derivatives are known in the art.

“Hybridization” generally depends on the ability of denatured DNA to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al, Current Protocols in Molecular Biology, Wiley Interscience Publishers (1995).

An “individual,” “subject” or “patient” is a vertebrate. In certain embodiments, the vertebrate is a mammal. Mammals include, but are not limited to, primates (including human and non-human primates), rodents (e.g., mice, hamsters, guinea pigs, and rats), farm animals, sport animals, and pets (e.g. dogs and cats). In certain embodiments, a mammal is a human. A “control subject” may refer to a healthy subject who has not been diagnosed as having a disease, dysfunction, or condition that has been identified in an individual, subject, or patient. The control subject does not suffer from any sign or symptom associated with the disease, dysfunction, or condition. Alternatively, a control subject may be a sick subject that does not receive the therapeutic drug, another drug, or a lower dose of the drug.

A “medicament” is an active drug that has been manufactured for the treatment of a disease, disorder, or condition.

“Morpholinos” are synthetic molecules that are non-natural variants of natural nucleic acids that utilize a phosphorodiamidate linkage, described in U.S. Pat. No. 8,076,476, incorporated by reference herein in its entirety.

“Multiple sclerosis” (MS) as used herein can include without limitation a heterogeneous condition consisting of recurrent and simultaneous inflammatory lesions of the spinal cord and brain causing demyelination of nerves in the central nervous system.

“Neuromyelitis optica” (NMO) as used herein can include without limitation a heterogeneous condition consisting of recurrent and simultaneous inflammation and demyelination of the optic nerve (optic neuritis) and spinal cord (myelitis). At least two different causes are known for NMO including the presence of autoantibodies against aquaporin 4 and aberrant astrocyte activity. An increasing body of evidence also supports the contribution of Th17 cells and a role of granulocytic release at the site of inflammation.

“Neuromyelitis optica” (NMO) as used herein can include without limitation a heterogeneous condition consisting of acute, recurrent or chronic/progressive inflammation or demyelination of the optic nerve (optic neuritis) or spinal cord (myelitis).

“Nucleic acids” are any of a group of macromolecules, either DNA, RNA, or variants thereof, that carry genetic information that may direct cellular functions. Nucleic acids may have enzyme-like activity (for instance ribozymes) or may be used to inhibit gene expression in a subject (for instance RNAi). The nucleic acids used in the inventions described herein may be single-stranded, double-stranded, linear or circular. The inventions further incorporate the use of nucleic acid variants including, but not limited to, aptamers, PNA, Morpholino, or other non-natural variants of nucleic acids. By way of example, nucleic acids useful for the invention are described in U.S. Pat. No. 8,076,476, incorporated by reference herein in its entirety.

“Patient response” or “response” can be assessed using any endpoint indicating a benefit to the patient, including, without limitation, (1) inhibition, to some extent, of disease progression, including stabilization, slowing down and complete arrest; (2) reduction in the number of disease episodes and/or symptoms; (3) inhibition (i.e., reduction, slowing down or complete stopping) of a disease cell infiltration into adjacent peripheral organs and/or tissues; (4) inhibition (i.e. reduction, slowing down or complete stopping) of disease spread; (5) decrease of an autoimmune condition; (6) favorable change in the expression of a biomarker associated with the disorder; (7) relief, to some extent, of one or more symptoms associated with a disorder; (8) increase in the length of disease-free presentation following treatment; or (9) decreased mortality at a given point of time following treatment.

As used herein, the term “peptide” is any peptide comprising two or more amino acids. The term peptide includes short peptides (e.g., peptides comprising between 2-14 amino acids), medium length peptides (15-50) or long chain peptides (e.g., proteins). The terms peptide, medium length peptide and protein may be used interchangeably herein. As used herein, the term “peptide” is interpreted to mean a polymer composed of amino acid residues, related naturally occurring structural variants, and synthetic non-naturally occurring analogs thereof linked via peptide bonds, related naturally-occurring structural variants, and synthetic non-naturally occurring analogs thereof. Synthetic peptides can be synthesized, for example, using an automated peptide synthesizer. Peptides can also be synthesized by other means such as by cells, bacteria, yeast or other living organisms. Peptides may contain amino acids other than the 20 gene-encoded amino acids. Peptides include those modified either by natural processes, such as processing and other post-translational modifications, but also by chemical modification techniques. Such modifications are well described in basic texts and in more detailed monographs, and are well-known to those of skill in the art. Modifications can occur anywhere in a peptide, including the peptide backbone, the amino acid side chains, and the amino or carboxyl termini.

As used herein, a “pharmaceutically acceptable carrier” or “therapeutic effective carrier” is aqueous or nonaqueous (solid), for example alcoholic or oleaginous, or a mixture thereof, and can contain a surfactant, emollient, lubricant, stabilizer, dye, perfume, preservative, acid or base for adjustment of pH, a solvent, emulsifier, gelling agent, moisturizer, stabilizer, wetting agent, time release agent, humectant, or other component commonly included in a particular form of pharmaceutical composition. Pharmaceutically acceptable carriers are well known in the art and include, for example, aqueous solutions such as water or physiologically buffered saline or other solvents or vehicles such as glycols, glycerol, and oils such as olive oil or injectable organic esters. A pharmaceutically acceptable carrier can contain physiologically acceptable compounds that act, for example, to stabilize or to increase the absorption of specific modulator(s), for example, carbohydrates, such as glucose, sucrose or dextrans, antioxidants such as ascorbic acid or glutathione, chelating agents, low molecular weight proteins or other stabilizers or excipients.

The term “pharmaceutical dose” or “pharmaceutical dosage form,” refers to physically discrete units suitable as unitary dosages for humans and other mammals, each unit comprising a predetermined quantity of agents in an amount calculated sufficient to produce the desired effect in association with an acceptable diluent, carrier, or vehicle of a formulation. The specifications for the unit dosage forms may depend on the particular serpin form employed, the effect to be achieved, the route of administration and the pharmacodynamics associated with the mammal.

“PNA” refers to peptide nucleic acids with a chemical structure similar to DNA or RNA. Peptide bonds are used to link the nucleotides or nucleosides together.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures.

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) overnight hybridization in a solution that employs 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μl/ml), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55° C.

As used herein, “treatment” refers to clinical intervention in an attempt to alter the natural course of the individual or cell being treated, and can be performed before or during the course of clinical pathology. Desirable effects of treatment include preventing the occurrence or recurrence of a disease or a condition or symptom thereof, alleviating a condition or symptom of the disease, diminishing any direct or indirect pathological consequences of the disease, decreasing the rate of disease progression, ameliorating or palliating the disease state, and achieving remission or improved prognosis. In some embodiments, methods and compositions of the invention are useful in attempts to delay development of a disease or disorder.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

In some embodiments, serpins and the other anti-inflammatory therapeutic compounds work synergistically. A person of skill in the art would appreciate that the synergistic activities may be established at appropriate fixed-dose ratios for efficacy against granulocyte-associated neuroinflammatory diseases. This may be determined by varying the amounts of two agents administered to appropriate animal models of inflammatory disease. The model may reflect either an active disease (following disease onset) or an early time point representative of pre-clinical disease. The effect on disease activity or progression is measured.

In other embodiments, the effects of varying amounts of the two agents may be determined in a cellular response mediating inflammation that may be involved in the pathogenesis of the disease. In other embodiments, the effects of varying amounts of the two agents in various formulations is determined by measuring enzymatic activity in vitro.

The presence, absence or degree of associated disease pathology or inflammatory markers such as granulocyte counts (in situ or circulating), protease activity, Th17 effector cell counts, plasma cell counts, CCR6, IL8, IL17, IL23, endogenous serpin, anti-aquaporin 4 antibodies, anti-Kir4 antibody levels can be used to evaluate efficacy. Successful compositions with the appropriate determined dose are selected that reduce said inflammatory markers, ameliorate disease symptoms or are tolerated by test animals.

The invention provides that the pharmaceutical compositions disclosed herein are formulated into solid, semi-solid, pressed powder, powder, liquid, gel, suspension, emulsion, or gaseous forms. In other embodiments, the pharmaceutical compositions are formulated into liquids, syrups, concentrates, tablets, capsules, caplets, powders, rapid melts, thin strips, granules, ointments, crémes, solutions, suspensions, emulsions, suppositories, injections, inhalants, gels, crystals and aerosols.

### Methods for Treating Neuroinflammatory Diseases

Individuals and other mammals at increased risk for development of a granulocyte-mediated neuroinflammatory disease, with early-stage of disease, or with established disease, may be treated with a clinically effective amount of any of the compositions disclosed herein. In some embodiments, the pharmaceutical compositions described herein prevent the development of disease, prevent the progression of disease, or to prevent the progression of the symptoms or signs of disease.

Thus, disclosed herein are methods for treating a patient with a neurodegenerative disease. Also disclosed herein are methods for treating a patient with symptoms consistent with neurodegenerative disease. Persons of skill in the art may determine preferred routes of administering the pharmaceutical compositions described herein, the corresponding dosage form, dose amount, and the dosing regimen (i.e., the frequency of dosing).

In some embodiments, the composition may be delivered in multi-dosing formats whereby the compositions are administered several times a week, once a day, twice a day, three times a day or more to achieve the appropriate therapeutic level. Other variables to consider include the specific serpin, inflammatory markers that are measured, the disease symptoms to be affected, the specific neuroinflammatory disease, the other anti-inflammatory therapeutic agent involved and its pharmacokinetic profile, and the specific individual involved.

In other embodiments, the frequency of administration may be once a month, once a week, once a day, or on an as-needed basis. Frequency of administration may be dependent on the identity and concentration of serpin in the composition or the disease risk assessment, disease severity, test results, clinician preference, or pharmaceutical formulation.

### Polynucleotides Encoding Serpins

Some embodiments of the invention provide serpin compositions for use as treatment for neuroinflammatory diseases. Said compositions may be administered on a daily, weekly, monthly, yearly or on an as-needed basis to reduce symptoms of disease or to reduce disease progression. In some embodiments, the serpin is A1AT.

The invention provides nucleic acids encoding a serpin protein. The nucleic acids may be DNA molecules, RNA molecules, aptamers (single-stranded or double-stranded), DNA or RNA oligonucleotides, larger DNA molecules that are linear or circular, oligonucleotides that are used for RNA interference (RNAi), variations of DNA such as substitution of DNA/RNA hybrid molecules, synthetic DNA-like molecules such as PNA or other nucleic acid derivative molecules (see WO07/035922, incorporated by reference herein in its entirety). In another embodiment, the therapeutic compound is composed of nuclease-resistant DNA or RNA oligonucleotides. In a preferred embodiment, nuclease-resistant DNA oligonucleotides are Morpholinos, (i.e. phosphorodiamidate analogs of nucleic acids that bind to nucleic acids in a sequence-specific manner, Sarepta Therapeutics, Cambridge Mass.).

In some embodiments, the serpin nucleic acids of the invention are synthesized using methods well-known in the art. In one embodiment, the nucleic acids are generated by enzymes. In exemplary embodiments, the enzymes may include DNA polymerases, RNA polymerases, ligases, and DNA repair enzymes. In another preferred embodiment, the nucleic acids are generated by a polymerase chain reaction (PCR) protocol. See, e.g. U.S. Pat. No. 4,683,195. In other embodiments, the nucleic acids are chemically synthesized using techniques well-known in the art. Typically, solid-phase nucleic acid synthesizers are used. Exemplary chemistries include phosphodiester synthesis, phosphotriester synthesis, and phosphite triester synthesis. See, e.g., Reese, Colin B. (2005). “Oligo- and poly-nucleotides: 50 years of chemical synthesis”. Organic & Biomolecular Chemistry 3 (21): 3851. The skilled artisan would understand that any techniques for synthesizing the nucleic acids and derivatives disclosed herein may be used.

In some embodiments, the serpin compositions of the invention may include A1AT. In a preferred embodiment, a nucleic acid containing at least about a 90% sequence identity to the human gene encoding A1AT precursor protein (SEQ ID NO:1) is delivered to a patient having neuroinflammatory symptoms consistent with NMO, MS and ALS. In another preferred embodiment, the A1AT composition may include a nucleic acid containing a sequence derived from A1AT mRNA. In another preferred embodiment, mRNA encoding human A1AT precursor protein is delivered to a patient having neuroinflammatory symptoms consistent with NMO, MS and ALS. In more preferred embodiments, the invention contemplates nucleic acids that hybridize with high stringency to a nucleic acid encoding A1AT (e.g. SEQ ID NO:2). Other preferred embodiments, nucleic acids encoding serpins are delivered to an individual via a viral vector, as a naked nucleic acid, or in a transformed cell.

In some embodiments, nucleic acids encoding serpins are administered to a patient in a cell-dependent manner. In preferred embodiments, the serpins or nucleic acids encoding them are delivered using transfected autologous patient cells. In other embodiments, serpins are delivered by intrathecal, intramuscular, intravascular, subcutaneous, intracranial, intraocular injection or inhaled routes. In more preferred embodiments, the nucleic acid encodes an A1AT protein having at least about a 90% sequence identity to SEQ ID NO:1. In more preferred embodiments, the serpin is an A1AT protein encoded by a nucleic acid that hybridize with high stringency to a nucleic acid encoding A1AT (e.g. SEQ ID NO:2).

The nucleic acids of the invention encode serpins that retain serpin functional activity. In preferred embodiments, the nucleic acids of the invention encode proteins that retain A1AT functional activity.

The invention provides non-viral serpin liquid or powder formulations. In some embodiments, the serpin is A1AT. In preferred embodiments, the serpin dose range is based on the selection of serpin form and associated properties. For example, plasmid backbone, promoter strength, and size, etc. In preferred embodiments, the copy number ranges from about 500 mM to about 10 pM per dose, depending on the use. Other embodiments comprise a serpin from about 500 mM to about 1 mM per dose. Further embodiments comprise a serpin from about 500 μM to about 1 μM per dose. Yet other embodiments comprise a serpin from about 10 μM to about 10 nM per dose. Further embodiments comprise a serpin from about 800 nM to about 10 pM per dose.

The invention provides viral serpin liquid or powder formulations. In preferred embodiments, the serpin is A1AT. Serpin dose can range based on selection of virus. Generally recommended are dose ranges from about 5×10⁹ PFU/mL to about 1×10³ PFU/mL per dose, depending on the use. Some compositions may comprise serpins from about 5×10⁹ PFU/mL to about 1×10⁸ PFU/mL per dose. Some compositions may comprise serpins from about 0.9×10⁸ PFU/mL to about 1×10⁶ PFU/mL per dose. Other compositions may comprise serpins from about 0.9×10⁶ PFU/mL to about 1×10⁵ PFU/mL per dose. Yet other compositions may comprise serpins from about 0.9×10⁵ PFU/mL to about 1×10³ PFU/mL per dose.

Periodicity of dosing may vary based on patient needs. In some embodiments, serpins are administered on a weekly or monthly basis. One advantage of a genetic approach is that serpin levels can be sustained longer than recombinant and human-derived purified forms.

Gene therapies and viral vectors that introduce DNA, RNA, transgenes, or other nucleic acid sequences to individuals are known in the art. (See, e.g., US20160046961; US20160040150; USRE045847; US20150218585; US20150167003; and U.S. Pat. No. 9,023,646; Rosenberg et al. N Engl. J. Med. 323: 570-8 (1990); Baltimore et al., Science 348: 36-8 (2015). The foregoing references are incorporated by reference in their entirety.)

The invention contemplates providing serpins using recombinant DNA techniques that result in addition or increased expression of a serpin. Exemplary technologies include homologous recombination, knock-in, ZFNs (zinc finger nucleases), TALENs (transcription activator-like effector nucleases), CRISPR (clustered regularly interspaced short palindromic repeats)/Cas9, and other site-specific nuclease technologies. These techniques enable double-strand DNA breaks at desired locus sites. These controlled double-strand breaks promote homologous recombination at the specific locus sites. This process relies on targeting specific sequences of nucleic acid molecules, such as chromosomes, with endonucleases that recognize and bind to the sequences and induce a double-stranded break in the nucleic acid molecule. The double-strand break is repaired either by an error-prone non-homologous end-joining (NHEJ) or by homologous recombination (HR). (See, e.g., WO2016025759, WO2015191693, US20160046961, US20160046960, US20160046952, US20160046915, and U.S. Pat. No. 9,260,752, each of which are incorporated by reference herein in their entirety.)

### Serpin Proteins

The invention provides therapeutic serpin peptides as disclosed herein. In one embodiment, the Serpin is a protein that has at least a 90% sequence identity to SEQ ID NO:1 and has A1AT activity. In preferred embodiments, the Serpin has the sequence of SEQ ID NO:1.

The terms “protein” and “peptide” refer to molecules that include a string of amino acids. The amino acids in the peptides of the invention may be naturally-occurring or non-naturally-occurring. The peptides of the invention may be synthesized chemically or biologically, and can include cysteine-rich peptides, circular peptides, stapled peptides, peptides that include D- or L-amino acids and mixtures thereof, peptidomimetics, peptide-nucleic acids (PNAs), and combinations thereof.

The invention provides recombinant or synthesized serpin compositions. In preferred embodiments, recombinant serpin compositions comprise cell-derived, purified serpins. In other preferred embodiments, human serpin precursor proteins are purified from an in vitro transfected cell culture.

In some embodiments, synthetic serpins are synthesized using protein chemistry known in the art. In preferred embodiments, the synthetic proteins are synthesized using liquid-phase or solid-phase peptide synthesis techniques.

Also contemplated within the scope of embodiments described herein are serpin peptides that are branched or cyclic, with or without branching. Cyclic, branched and branched circular peptides result from post-translational natural processes and are also made by suitable synthetic methods. In some embodiments, any peptide product described herein comprises a peptide analog described above that is then covalently attached to an alkyl-glycoside surfactant moiety.

Other embodiments include serpin peptide chains that are comprised of natural and unnatural amino acids or analogs of natural amino acids. As used herein, peptide and/or protein “analogs” comprise non-natural amino acids based on natural amino acids, such as tyrosine analogs, which includes para-substituted tyrosines, ortho-substituted tyrosines, and meta-substituted tyrosines, wherein the substituent on the tyrosine comprises an acetyl group, a benzoyl group, an amino group, a hydrazine, an hydroxyamine, a thiol group, a carboxy group, a methyl group, an isopropyl group, a C2-C20 straight chain or branched hydrocarbon, a saturated or unsaturated hydrocarbon, an O-methyl group, a polyether group, a halogen, a nitro group, or the like.

Additional embodiments include serpin peptide chains having modified amino acids.

Examples include acylated amino acids at the ε-position of Lysine, amino acids with fatty acids such as octanoic, decanoic, dodecanoic, tetradecanoic, hexadecanoic, octadecanoic, 3-phenylpropanoic acids and the like, or with saturated or unsaturated alkyl chains. (Zhang, L. and Bulaj, G (2012) Curr Med Chem 19: 1602-1618, incorporated herein by reference in its entirety).

The invention further contemplates serpin peptide chains comprising natural and unnatural amino acids or analogs of natural amino acids. In some embodiments, peptide or protein “analogs” comprise non-natural amino acids based on natural amino acids, such as tyrosine analogs, which includes para-substituted tyrosines, ortho-substituted tyrosines, and meta-substituted tyrosines, wherein the substituent on the tyrosine comprises an acetyl group, a benzoyl group, an amino group, a hydrazine, an hydroxyamine, a thiol group, a carboxy group, a methyl group, an isopropyl group, a C2-C20 straight chain or branched hydrocarbon, a saturated or unsaturated hydrocarbon, an O-methyl group, a polyether group, a halogen, a nitro group, or the like. Examples of Tyr analogs include 2,4-dimethyl-tyrosine (Dmt), 2,4-diethyl-tyrosine, O-4-allyl-tyrosine, 4-propyl-tyrosine, Ca-methyl-tyrosine and the like. Examples of lysine analogs include ornithine (Orn), homo-lysine, Ca-methyl-lysine (CMeLys), and the like. Examples of phenylalanine analogs include, but are not limited to, meta-substituted phenylalanines, wherein the substituent comprises a methoxy group, a C1-C20 alkyl group, for example a methyl group, an allyl group, an acetyl group, or the like. Specific examples include, but are not limited to, 2,4,6-trimethyl-L-phenylalanine (Tmp), O-methyl-tyrosine, 3-(2-naphthyl)alanine (Nal(2)), 3-(1-naphthyl)alanine (Nal(1)), 3-methyl-phenylalanine, 1,2,3,4-tetrahydroisoquinoline-3-carboxylic acid (Tic), fluorinated phenylalanines, isopropyl-phenylalanine, p-azido-phenylalanine, p-acyl-phenylalanine, p-benzoyl-phenylalanine, p-iodo-phenylalanine, p-bromophenylalanine, p-amino-phenylalanine, and isopropyl-phenylalanine, and the like.

Also contemplated within the scope of embodiments are therapeutic peptide chains containing nonstandard or unnatural amino acids known to the art, for example, C-alpha-disubstituted amino acids such as Aib, Ca-diethylglycine (Deg), aminocyclopentane-1-carboxylic acid (Ac4c), aminocyclopentane-1-carboxylic acid (Ac5c), and the like. Such amino acids frequently lead to a restrained structure, often biased toward an alpha helical structure (Kaul, R. and Balaram, P. (1999) Bioorg Med Chem 7: 105-117, incorporated herein by reference in its entirety). Additional examples of such unnatural amino acids useful in analog design are homo-arginine (Har) and the like. Substitution of reduced amide bonds in certain instances leads to improved protection from enzymatic destruction or alters receptor binding. By way of example, incorporation of a Tic-Phe dipeptide unit with a reduced amide bond between the residues (designated as Tic-F[CH2-NH]̂-Phe) reduces enzymatic degradation.

In some embodiments, modifications at the amino or carboxyl terminus may optionally be introduced into the present peptides or proteins (Nestor, J. J., Jr. (2009) Current Medicinal Chemistry 16: 4399-4418). For example, the present peptides or proteins can be truncated or acylated on the N-terminus (Gourlet, P., et al. (1998) Eur J Pharmacol 354: 105-1 1 1, Gozes, I. and Furman, S. (2003) Curr Pharm Des 9: 483-494), the contents of which is incorporated herein by reference in their entirety). Other modifications to the N-terminus of peptides or proteins, such as deletions or incorporation of D-amino acids such as D-Phe result in potent and long acting agonists or antagonists when substituted with the modifications described herein such as long chain alkyl glycosides.

Thus, the invention provides serpin compound analogs wherein the native therapeutic compound is modified by acetylation, acylation, PEGylation, ADP-ribosylation, amidation, covalent attachment of a lipid or lipid derivative, covalent attachment of phosphotidylinositol, cross-linking, cyclization, disulfide bond formation, demethylation, formation of covalent cross-link formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, (Nestor, J. J., Jr. (2007) Comprehensive Medicinal Chemistry II 2: 573-601, Nestor, J. J., Jr. (2009) Current Medicinal Chemistry 16: 4399-4418, Uy, R. and Wold, F. (1977) Science 198:890-6, Seifter, S. and Englard, S. (1990) Methods Enzymol 182: 626-646, Rattan, S. I., et al. (1992) Ann NY Acad Sci 663: 48-62). The foregoing references are incorporated by reference in their entirety.

Glycosylated serpin peptides may be prepared using conventional Fmoc chemistry and solid phase peptide synthesis techniques, e.g., on resin, where the desired protected glycoamino acids are prepared prior to peptide synthesis and then introduced into the peptide chain at the desired position during peptide synthesis. Thus, the therapeutic peptide polymer conjugates may be conjugated in vitro. The glycosylation may occur before deprotection. Preparation of amino acid glycosides is described in U.S. Pat. No. 5,767,254, WO 2005/097158, and Doores, K., et al., Chem. Commun., 1401-1403, 2006, which are incorporated herein by reference in their entirety. For example, alpha and beta selective glycosylations of serine and threonine residues are carried out using the Koenigs-Knorr reaction and Lemieux's in situ anomerization methodology with Schiff base intermediates. Deprotection of the Schiff base glycoside is then carried out using mildly acidic conditions or hydrogenolysis. A composition, comprising a glycosylated therapeutic peptide conjugate is made by stepwise solid phase peptide synthesis involving contacting a growing peptide chain with protected amino acids in a stepwise manner, wherein at least one of the protected amino acids is glycosylated, followed by water-soluble polymer conjugation. Such compositions may have a purity of at least 95%, at least 97%, or at least 98%, of a single species of the glycosylated and conjugated therapeutic peptide.

Monosaccharides that may by used for introduction at one or more amino acid residues of the therapeutic peptides defined and/or disclosed herein include glucose (dextrose), fructose, galactose, and ribose. Additional monosaccharides suitable for use include glyceraldehydes, dihydroxyacetone, erythrose, threose, erythrulose, arabinose, lyxose, xylose, ribulose, xylulose, allose, altrose, mannose, N-Acetylneuraminic acid, fucose, N-Acetylgalactosamine, and N-Acetylglucosamine, as well as others. Glycosides, such as mono-, di-, and trisaccharides for use in modifying a therapeutic peptide, one or more amino acid residues of the therapeutic peptides defined and/or disclosed herein include sucrose, lactose, maltose, trehalose, melibiose, and cellobiose, among others. Trisaccharides include acarbose, raffinose, and melezitose.

In other embodiments, the nucleic acids of the invention may be expressed in microorganisms. Promoters for expressing genes of interest are known in the art. In some embodiments, the expression vectors of the invention may have promoters, transcription terminators, or selectable markers. Either inducible or constitutive promoters are contemplated by the invention.

In a preferred embodiment, the nucleic acids of the invention are expressed in bacterial systems because of their low cost, high productivity, and rapid use. Thus, the nucleic acids are expressed in, for example, Bacillus brevis, Bacillus megaterium, Bacillus subtilis, Caulobacter crescentus, Escherichia coli and their derivatives. Exemplary promoters include the 1-arabinose inducible araBAD promoter (PBAD), the lac promoter, the 1-rhamnose inducible rhaP BAD promoter, the T7 RNA polymerase promoter, the trc and tac promoter, the lambda phage promoter pL, and the anhydrotetracycline-inducible tetA promoter/operator.

In some embodiments, the nucleic acids of the invention are expressed in yeast expression systems. Exemplary promoters used in yeast vectors include the promoters for 3-phosphoglycerate kinase (Hitzeman et al., J. Biol. Chem. 255:2073 ((1980)); and other glycolytic enzymes (Hess et al., J. Adv. Enzyme Res. 7:149 (1968); Holland et al., Biochemistry 17:4900 (1978)), e.g., enolase, glyceraldehyde-3-phosphate dehydrogenase, hexokinase, pyvurate decarboxylase, phosphofructokinase, glucose-6-phosphate isomerase, 3-phosphoglycerate mutase, pyruvate kinase, triosephosphate somerase, phosphoglucose isomerase, glucokinase alcohol oxidase I (AOX1), alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing a yeast-compatible promoter and termination sequences, with or without an origin of replication, is suitable. Yeast expression systems are commercially available, for example, from Clontech Laboratories, Inc. (Palo Alto, Calif., e.g. pYEX 4T family of vectors for S. cerevisiae), Invitrogen (Carlsbad, Calif., e.g. pPICZ series Easy Select Pichia Expression Kit) and Stratagene (La Jolla, Calif., e.g. ESP™ Yeast Protein Expression and Purification System for S. pombe and pESC vectors for S. cerevisiae).

In other embodiments, the nucleic acids of the invention are expressed in mammalian expression systems. Examples of suitable mammalian promoters for use in the invention include, for example, promoters from the following genes: ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s). In a preferred embodiment, a yeast alcohol oxidase promoter is used.

In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication. Fiers et al., Nature 273: 113-120 (1978). The immediate early promoter of the human cytomegalovirus is conveniently obtained as a HindIII E restriction fragment. Greenaway, P. J. et al., Gene 18: 355-360 (1982). The foregoing references are incorporated by reference in their entirety.

In some embodiments, the nucleic acids of the invention are expressed in insect cell expression systems. Eukaryotic expression systems employing insect cell hosts may rely on either plasmid or baculoviral expression systems. The typical insect host cells are derived from the fall army worm (Spodoptera frugiperda). For expression of a foreign protein these cells are infected with a recombinant form of the baculovirus Autographa californica nuclear polyhedrosis virus which has the gene of interest expressed under the control of the viral polyhedrin promoter. Other insects infected by this virus include a cell line known commercially as “High 5” (Invitrogen) which is derived from the cabbage looper (Trichoplusia ni). Another baculovirus sometimes used is the Bombyx mori nuclear polyhedorsis virus which infect the silk worm (Bombyx mori). Numerous baculovirus expression systems are commercially available, for example, from Invitrogen (Bac-N-Blue™), Clontech (BacPAK™ Baculovirus Expression System), Life Technologies (BAC-TO-BAC™), Novagen (Bac Vector System™), Pharmingen and Quantum Biotechnologies). Another insect cell host is the common fruit fly, Drosophila melanogaster, for which a transient or stable plasmid based transfection kit is offered commercially by Invitrogen (The DES™ System).

In some embodiments, cells are transformed with vectors that express the nucleic acids of the invention. Transformation techniques for inserting new genetic material into eukaryotic cells, including animal and plant cells, are well known. Viral vectors may be used for inserting expression cassettes into host cell genomes. Alternatively, the vectors may be transfected into the host cells. Transfection may be accomplished by calcium phosphate precipitation, electroporation, optical transfection, protoplast fusion, impalefection, and hydrodynamic delivery.

In preferred embodiments, the serpin nucleic acids are expressed in mammalian cell lines that are well-known in the art. Exemplary mammalian cell lines include Chinese hamster ovary cells (CHO) and Vero cells. The serpins are recovered using known biochemical and biologics manufacturing techniques. (See, e.g., Lai et al., Pharmaceuticals 6:579-603 (2013), incorporated by reference herein in its entirety.)

Examplary therapeutic Serpin family members are presented in Table 1.

TABLE 1

| SERPIN | SOURCE | SEQ ID NO | DEMONSTRATED EFFECTS | DOSE RANGE (per kg) |
|---|---|---|---|---|
| A1AT | Human | 1, 2 | Inhibits neutrophil elastase | 1,000 mg to 0.5 ng; |
| (SerpinA1) | | | and other proteases | 500 mM to 10 pM |
| CrmA | Cowpox | 3, 4 | Suppressor of IL1 and IL18 | 1,000 mg to 0.5 ng; |
| | virus | | | 300 mM to 10 pM |
| Serpin1 or | Arabidopsis | 5, 6 | Suppressor of metacaspases | 2,000 mg to 10 ng; |
| AtSerpin1 or | | | and papain-like cysteine | 1000 mM to 50 pM |
| Serpin-ZX | | | protease | |
| SerpinB9 | Human | 7, 8 | Inhibits Granzyme B | 1,000 mg to 0.5 ng; |
| | | | | 500 mM to 10 pM |
| SerpinA3 | Human | 9, 10, 11 | Inhibits cathepsin G | 1,000 mg to 0.5 ng; |
| | | | | 500 mM to 10 pM |
| SerpinA4 | Human | 12-17 | Inhibits kallikrein | 1,000 mg to 0.5 ng; |
| | | | | 500 mM to 10 pM |
| SerpinA5 | Human | 18, 19 | Inhibits protein C, reduces | 1,000 mg to 0.5 ng; |
| | | | PCI in MS plaques | 500 mM to 10 pM |
| SerpinA9 | Human | 20-27 | Inhibits B cell activation | 2,000 mg to 1.5 ng; |
| | | | | 700 mM to 50 pM |
| SerpinA10 | Human | 28-31 | Inhibits protein Z-related | 1,000 mg to 0.5 ng; |
| | | | protease, factors Xa and XIa | 500 mM to 10 pM |
| SerpinA12 | Human | 32-35 | Inhibits Kallikrein-7 | 1,000 mg to 0.5 ng; |
| | | | | 500 mM to 10 pM |
| SerpinB1 | Human | 36, 37 | Inhibits neutrophil elastase | 1,000 mg to 0.5 ng; |
| | | | and monocytes | 500 mM to 10 pM |
| SerpinB6 | Human | 38-53 | Inhibits cathepsin G | 1,000 mg to 0.5 ng; |
| | | | | 500 mM to 10 pM |
| SerpinB9 | Human | 54, 55 | Inhibits cytotoxic granule | 2,000 mg to 5 ng; |
| | | | proteases such as granzyme B | 1000 mM to 10 pM |
| Serping1 | Human | 56-59 | C1 esterase inhibitor | 1,000 mg to 0.5 ng; |
| | | | | 500 mM to 10 pM |
| Serpini1 | Human | 60-63 | Inhibits tPA, uPA and plasmin, | 2,000 mg to 5 ng; |
| (neuroserpin) | | | mutated in dementia | 1000 mM to 10 pM |

### Formulations

The invention provides that any of the serpin compositions disclosed herein are formulated into a pharmaceutical composition. In some embodiments, a serpin and an anti-inflammatory therapeutic compound are the only active ingredients. In other embodiments, they are formulated with one or more additional active ingredients. In preferred embodiments, the combinatorial pharmaceutical compositions comprise steroids such as prednisolone or prednisone, anti-inflammatory compounds such as doxycycline, tetracycline, intravenous immunoglobulin, non-steroidal anti-inflammatory drugs (NSAID) such as celecoxib, indomethacin, naproxen, ibuprofen, acetaminophen, and rofecoxib.

The invention provides pharmaceutical compositions formulated with pharmaceutically acceptable excipients, carriers, diluents or vehicles. In some embodiments, they are formulated in powders, liquids, gels, pastes, suspensions, emulsions, or gaseous forms. In other embodiments, they are formulated into pharmaceutically acceptable dosage forms such as: tablets, capsules, caplets, powders, granules, ointments, crémes, solutions, suspensions, emulsions, suppositories, injections, inhalants, gels, particles, or aerosols. In other embodiments, the formulations are administered as disclosed herein. In other embodiments, serpins are administered in a free form, as pharmaceutically acceptable salts, in a time-release formulation, sequentially in a discrete manner, or in combination with other pharmaceutically active compounds.

In some embodiments, the serpins of the invention are delivered to a patient by intrathecal, intramuscular, intravascular, subcutaneous, intracranial, or intraocular injection. In a preferred embodiment, the serpin is A1AT. In another preferred embodiment, the serpins are provided in liquid and powder formulations at amounts ranging from about 1,000 mg/kg to about 50 ng/kg per dose, depending on the method of administration, potency and use. Some formulations may comprise recombinant serpins from about 1,000 mg to about 50 mg per dose. Some formulations may comprise recombinant serpins from about 75 mg to about 5 mg per dose. Some formulations may comprise recombinant serpins from about 5 mg to about 100 μg per dose. Other formulations may comprise recombinant serpins from about 150 μg to about 8 μg per dose. Yet other formulations comprise recombinant serpins from about 7.5 μg to about 50 ng per dose. In preferred embodiments, the formulated serpin is A1AT.

In other embodiments, the periodicity of dosing varies based on patient needs. In preferred embodiments, the dosing schedule is approximately: multiple times per day, daily, multiple times per week, weekly, bi-weekly, monthly, every 6 weeks, every two months, every three months, every four months, every five months, every 6 months, annually, or on an as-needed basis. In a preferred embodiment, the serpin is A1AT. In a more preferred embodiment 60 mg/kg is administered weekly

In other embodiments, the serpin is a human A1AT that is, for example, Prolastin-C (Grifols USA, Los Angeles, Calif.), Aralast NP (Baxter Healthcare Corp., Westlake Village, Calif.), Glassia (Baxter Healthcare Corp., Westlake Village, Calif.) and Zemaira (CSL Behring, King of Prussia, Pa.). In other embodiments, A1AT formulations derived from human blood comprise liquid and powder formulations at amounts ranging from about 1,000 mg/kg to about 50 ng/kg per dose, depending on the method of administration, potency and use. In other embodiments, the formulations comprise Recombinant A1AT from about 1,000 mg to about 50 mg per dose. In other embodiments, formulations comprise recombinant A1AT from about 75 mg to about 5 mg per dose. In other embodiments, formulations comprise Recombinant A1AT from about 5 mg to about 100 μg per dose. In other embodiments, formulations comprise recombinant A1AT from about 150 μg to about 8 μg per dose. In other embodiments, formulations comprise Recombinant A1AT from about 7.5 μg to about 50 ng per dose.

Exemplary drug formulations of the invention include aqueous solutions, organic solutions, powder formulations, solid formulations and mixed phase formulations.

Pharmaceutical compositions of this invention comprise any of the compounds of the present invention, and pharmaceutically acceptable salts thereof, with any pharmaceutically acceptable carrier, adjuvant or vehicle. Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutically acceptable salts retain the desired biological activity of the therapeutic composition without toxic side effects. Examples of such salts are (a) acid addition salts formed with inorganic acids, for example, hydrochloric acid, hydrobromic acid, sulfuric acid, phosphoric acid, nitric acid and the like/and salts formed with organic acids such as, for example, acetic acid, trifluoroacetic acid, tartaric acid, succinic acid, maleic acid, fumaric acid, gluconic acid, citric acid, malic acid, ascorbic acid, benzoic acid, tanic acid, pamoic acid, alginic acid, polyglutamic acid, naphthalenesulfonic acid, naphthalene disulfonic acid, polygalacturonic acid and the like; (b) base addition salts or complexes formed with polyvalent metal cations such as zinc, calcium, bismuth, barium, magnesium, aluminum, copper, cobalt, nickel, cadmium, and the like; or with an organic cation formed from N,N′-dibenzylethylenediamine or ethlenediamine; or (c) combinations of (a) and (b), e.g. a zinc tannate salt and the like.

The pharmaceutical compositions of this invention may be administered by subcutaneous, transdermal, oral, parenteral, inhalation, ocular, topical, rectal, nasal, buccal (including sublingual), vaginal, or implanted reservoir modes. The pharmaceutical compositions of this invention may contain any conventional, non-toxic, pharmaceutically-acceptable carriers, adjuvants or vehicles. The term parenteral as used herein includes subcutaneous, intracutaneous, intravenous, intramuscular, intraarticular, intrasynovial, intrastemal, intrathecal, intralesional, and intracranial injection or infusion techniques.

Also contemplated, in some embodiments, are pharmaceutical compositions comprising as an active ingredient, therapeutic compounds described herein, or pharmaceutically acceptable salt thereof, in a mixture with a pharmaceutically acceptable, non-toxic component. As mentioned above, such compositions may be prepared for parenteral administration, particularly in the form of liquid solutions or suspensions; for oral or buccal administration, particularly in the form of tablets or capsules; for intranasal administration, particularly in the form of powders, nasal drops, evaporating solutions or aerosols; for inhalation, particularly in the form of liquid solutions or dry powders with excipients, defined broadly; for transdermal administration, particularly in the form of a skin patch or microneedle patch; and for rectal or vaginal administration, particularly in the form of a suppository.

The compositions may conveniently be administered in unit dosage form and may be prepared by any of the methods well-known in the pharmaceutical art, for example, as described in Remington's Pharmaceutical Sciences, 17th ed., Mack Publishing Co., Easton, Pa. (1985), incorporated herein by reference in its entirety. Formulations for parenteral administration may contain as excipients sterile water or saline alkylene glycols such as propylene glycol, polyalkylene glycols such as polyethylene glycol, saccharides, oils of vegetable origin, hydrogenated napthalenes, serum albumin or other nanoparticles (as used in Abraxane™, American Pharmaceutical Partners, Inc. Schaumburg, Ill.), and the like. For oral administration, the formulation can be enhanced by the addition of bile salts or acylcarnitines. Formulations for nasal administration may be solid or solutions in evaporating solvents such as hydrofluorocarbons, and may contain excipients for stabilization, for example, saccharides, surfactants, submicron anhydrous alpha-lactose or dextran, or may be aqueous or oily solutions for use in the form of nasal drops or metered spray. For buccal administration, typical excipients include sugars, calcium stearate, magnesium stearate, pregelatinated starch, and the like.

Delivery of modified therapeutic compounds described herein to a subject over prolonged periods of time, for example, for periods of one week to one year, may be accomplished by a single administration of a controlled release system containing sufficient active ingredient for the desired release period. Various controlled release systems, such as monolithic or reservoir-type microcapsules, depot implants, polymeric hydrogels, osmotic pumps, vesicles, micelles, liposomes, transdermal patches, iontophoretic devices and alternative injectable dosage forms may be utilized for this purpose. Localization at the site to which delivery of the active ingredient is desired is an additional feature of some controlled release devices, which may prove beneficial in the treatment of certain disorders.

In certain embodiments for transdermal administration, delivery across the barrier of the skin would be enhanced using electrodes (e.g. iontophoresis), electroporation, or the application of short, high-voltage electrical pulses to the skin, radiofrequencies, ultrasound (e.g. sonophoresis), microprojections (e.g. microneedles), jet injectors, thermal ablation, magnetophoresis, lasers, velocity, or photomechanical waves. The drug can be included in single-layer drug-in-adhesive, multi-layer drug-in-adhesive, reservoir, matrix, or vapor style patches, or could utilize patchless technology. Delivery across the barrier of the skin could also be enhanced using encapsulation, a skin lipid fluidizer, or a hollow or solid microstructured transdermal system (MTS, such as that manufactured by 3M), jet injectors. Additives to the formulation to aid in the passage of therapeutic compounds through the skin include prodrugs, chemicals, surfactants, cell penetrating peptides, permeation enhancers, encapsulation technologies, enzymes, enzyme inhibitors, gels, nanoparticles and peptide or protein chaperones.

One form of controlled-release formulation contains the therapeutic compound or its salt dispersed or encapsulated in a slowly degrading, non-toxic, non-antigenic polymer such as copoly(lactic/glycolic) acid, as described in the pioneering work of Kent et al., U.S. Pat. No. 4,675,189, incorporated by reference herein. The compounds, or their salts, may also be formulated in cholesterol or other lipid matrix pellets, or silastomer matrix implants. Additional slow release, depot implant or injectable formulations will be apparent to the skilled artisan. See, for example, Sustained and Controlled Release Drug Delivery Systems, JR Robinson ed., Marcel Dekker Inc., New York, 1978; and Controlled Release of Biologically Active Agents, R W Baker, John Wiley & Sons, New York, 1987. The foregoing are incorporated by reference in their entirety.

An additional form of controlled-release formulation comprises a solution of biodegradable polymer, such as copoly(lactic/glycolic acid) or block copolymers of lactic acid and PEG, is a bioacceptable solvent, which is injected subcutaneously or intramuscularly to achieve a depot formulation. Mixing of the therapeutic compounds described herein with such a polymeric formulation is suitable to achieve very long duration of action formulations.

When formulated for nasal administration, the absorption across the nasal mucous membrane may be further enhanced by surfactants, such as, for example, glycocholic acid, cholic acid, taurocholic acid, ethocholic acid, deoxycholic acid, chenodeoxycholic acid, dehdryocholic acid, glycodeoxycholic acid, cycledextrins and the like in an amount in the range of between about 0.1 and 15 weight percent, between about 0.5 and 4 weight percent, or about 2 weight percent. An additional class of absorption enhancers reported to exhibit greater efficacy with decreased irritation is the class of alkyl maltosides, such as tetradecylmaltoside (Arnold, J J et al., 2004, J Pharm Sci 93: 2205-13; Ahsan, F et al., 2001, Pharm Res 18:1742-46) and references therein, all of which are hereby incorporated by reference.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as Ph. Helv or a similar alcohol.

The pharmaceutical compositions of this invention may be orally administered in any orally acceptable dosage form including, but not limited to, capsules, tablets, and aqueous suspensions and solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient that is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topical transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

When formulated for delivery by inhalation, a number of formulations offer advantages. Adsorption of the therapeutic compound to readily dispersed solids such as diketopiperazines (for example, Technosphere particles (Pfutzner, A and Forst, T, 2005, Expert Opin Drug Deliv 2:1097-1106) or similar structures gives a formulation that results in rapid initial uptake of the therapeutic compound. Lyophilized powders, especially glassy particles, containing the therapeutic compound and an excipient are useful for delivery to the lung with good bioavailability, for example, see Exubera® (inhaled insulin, Pfizer, Inc. and Aventis Pharmaceuticals Inc.) and Afrezza® (inhaled insulin, Mannkind, Corp.).

Dosage levels of between about 0.01 and about 100 mg/kg body weight per day, preferably 0.5 and about 50 mg/kg body weight per day of the active ingredient compound are useful in the prevention and treatment of disease. Such administration can be used as a chronic or acute therapy. The amount of drug that may be combined with the carrier to produce a single dosage form will vary depending upon the host treated and the particular mode of administration. A typical preparation will contain from about 5% to about 95% active compound (w/w). Preferably, such preparations contain from about 20% to about 80% active compound.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level, treatment should cease. Patients may, however, require intermittent treatment on a long-term basis upon any recurrence of disease symptoms.

As the skilled artisan will appreciate, lower or higher doses than those recited above may be required. Specific dosage and treatment regimens for any particular patient will depend upon a variety of factors, including the activity of the specific compound employed, the age, body weight, general health status, gender, diet, time of administration, rate of excretion, drug combination, the severity and course of an infection, the patient's disposition to the infection and the judgment of the treating physician.

The carrier-drug conjugates described herein provide advantages to drug manufacturers and patients over unmodified drugs. Specifically, the carrier-drug conjugate or formulation will be a more potent, longer lasting, and require smaller and less frequent dosing. This translates into lowered healthcare costs and more convenient drug administration schedules for patients. The carrier-drug conjugates can also provide subcutaneous or transdermal routes of administration as alternatives to intravenous injection. These routes can be self-administered by patients and thus improve patient compliance.

In order that the invention described herein may be more fully understood, the following examples are set forth. It should be understood that these examples are for illustrative purposes only and are not to be construed as limiting this invention in any manner.

### Example 1—A1AT Reduces NMO Disease in a Mouse Model

C57BL/6 donor mice were immunized via subcutaneous injection with MOG-35-55 peptide in complete Freund's Adjuvant. This induces Experimental Autoimmune Encephalomyelitis (EAE), an animal model for progressive multiple sclerosis. (Miller et al., Curr. Protoc. Immunol., Chapter Unit 15.1 (2007); incorporated herein by reference in its entirety). The mice were boosted twice via intraperitoneal injection with Pertussis toxin on day 0, 2, and 7 days post-immunization. Mice were sacrificed and spleens and lymph nodes collected and resuspended into single cell suspensions. Cells were cultured with MOG peptide and IL-23 to increase Th17 skewing and cultured for 3 days. Cells were harvested and approximately 15×10̂6 cells were adoptively transferred into recipient C57BL/6 mice via intraperitoneal injection. Starting day 5 for 2 weeks until day 18, mice were administered daily via intraperitoneal injection with 100 uL of PBS or A1AT (2 mg A1AT formulated in PBS). Clinical scores were measured each day at approximately the same time of day. Clinical scores measure ascending paralysis. A score of 1 is a limp tail. A score of 2 includes hind limb weakness. A score of 3 includes hind limb paralysis. A score of 4 includes forelimb weakness. A score of 5 is indicated by death. (See Miller et al.). A1AT showed a significant improvement when compared to PBS treatment. n=10 mice per group.

### Example 2—A1AT Reduces NMO Disease in a Mouse Model

Blood-derived human A1AT was compared to Sivelestat in a murine model of NMO. A less severe disease was induced when 12×10⁶ Th17-polarized cells were adoptively transferred i.p. into recipient C57BL/6 mice. To induce Th17-polarized cells, C57BL/6 mice were induced with MOG₃₅₋₅₅/CFA. 11 days later, their spleen cells were harvested and restimulated in culture with MOG₃₅₋₅₅ peptide (Miller et al., supra) for 3 days.

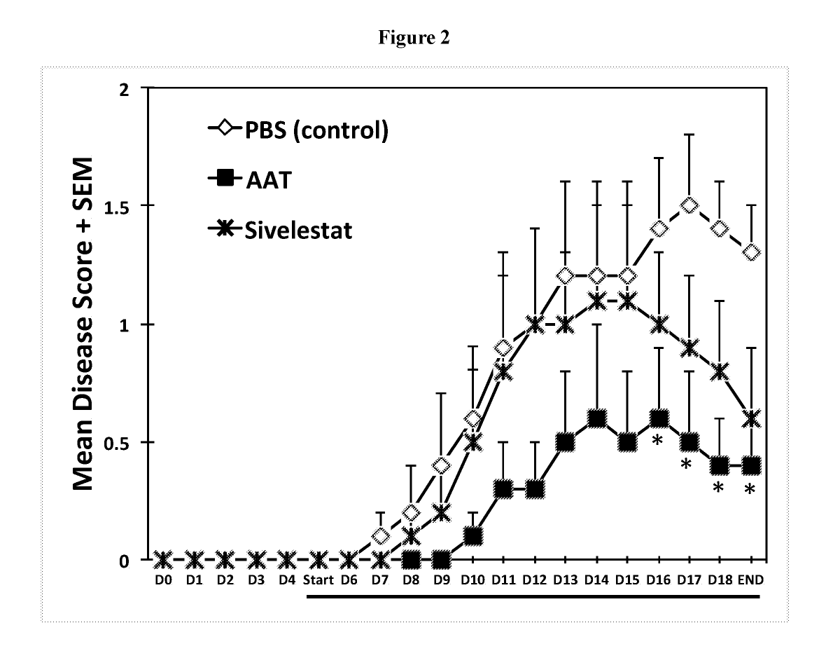

Mice received PBS, Sivelestat (0.5 mg), or A1AT (2 mg) as daily doses administered i.p. starting on Day 5 post-transfer until Day 18 post-transfer. There were 10 recipient mice per cohort. Error bars represent SEM. Points where A1AT compared to PBS and mean disease scores were significant (p<0.05) are indicated (*). A1AT treatment reduced the NMO disease score compared to PBS (control) and was superior to Sivelestat at all days of overt signs of disease as well as delay onset of disease (see FIG. 2).

### Example 3—A1AT+Methylprednisolone Reduces NMO Disease in a Mouse Model

A more severe disease was induced by adoptively transferring a higher number of pathogenic Th17-polarized cells in order to demonstrate the additional efficacy from a combination treatment of A1AT plus methylprednisolone (Mpred) compared to individual treatments using a blinded study.

25×10⁶ cells prepared as above were adoptively transferred i.p. into recipient C57BL/6 mice. Mice received PBS, Mpred (0.5 mg), AAT (2 mg), or both AAT+Mpred as daily doses administered i.p. starting on Day 4 post-transfer until Day 16 post-transfer. Ten recipient mice per cohort. Table 2 shows the Mean day of disease onset+/−SEM and p-value determined by the two-tailed Student's t-test; the end disease score at Day 17 post-transfer+/−SD, p-value determined by Mann-Whitney U test; and the mean maximum disease score (MMS)+/−SD, p-value by Mann-Whitney U test. The bold p-values indicate significant (p<0.05) differences between the treatments when compared to PBS (control).

The AAT+Mpred combination demonstrated superior and significant delay of disease onset, showed improved disease end scores equivalent to Mpred alone, and improved mean maximum disease score (MMS) equivalent to Mpred alone.

TABLE 2

| | Mean Day of | | | | | |
|---|---|---|---|---|---|---|
| Treatment | Onset +/− SEM | p-value | End Score | p-value | MMS +/− SD | p-value |
| PBS | 7.9 +/− 0.3 | | 3.00 +/− 1.11 | | 3.15 +/− 1.11 | |
| Mpred | 8.5 +/− 0.4 | 0.2520 | 2.25 +/− 0.42 | 0.0039 | 2.95 +/− 0.44 | 0.0228 |
| A1AT | 8.4 +/− 0.4 | 0.3110 | 3.10 +/− 0.57 | 0.6890 | 3.50 +/− 0.00 | 0.3173 |
| A1AT + Mpred | 9.3 +/− 0.5 | 0.0164 | 2.15 +/− 1.00 | 0.0150 | 2.70 +/− 1.06 | 0.0447 |

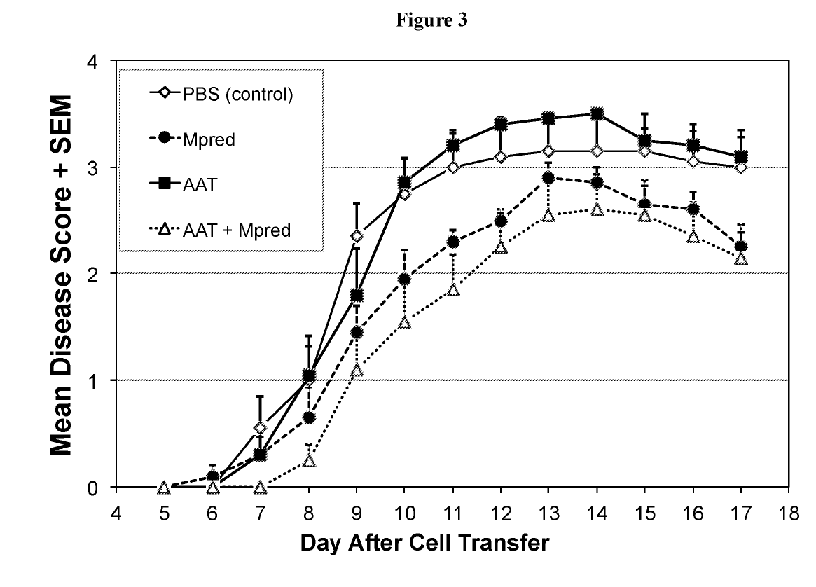

Mean disease scores showed that A1AT+Mpred treatment reduced the disease over all days measured more than either treatment alone (see FIG. 3). AAT alone showed early efficacy compared to Mpred alone and PBS (control) on Day 5 and 6 but afterwards did not show therapeutic effect. While not being bound to theory, this may be due to anti-drug immunogenicity of the human AAT in mouse that is known to develop (70) that may not have developed in our previous less severe disease induction, and where the Mpred in combination with AAT treatment may have diminished the anti-drug immunogenicity effect.

Histological analysis showed that A1AT+Mpred and A1AT alone reduced the number of inflammatory foci compared to Mpred alone. Additionally, A1AT+Mpred similarly reduced demyelination and the number of apoptotic cells when compared to Mpred alone.

TABLE 3

| | Inflammatory | | Demyelination | | Apoptotic | |
|---|---|---|---|---|---|---|
| Treatment | foci +/− SD | p-value | (H + E) | p-value | Cells +/− SD | p-value |
| PBS | 3.6 +/− 0.5 | | 1.8 +/− 0.2 | | 2.9 +/− 0.9 | |
| Mpred | 3.0 +/− 0.9 | 0.2856 | 1.2 +/− 0.3 | 0.0325 | 1.3 +/− 0.9 | 0.0393 |
| A1AT | 2.0 +/− 0.5 | 0.0037 | 1.8 +/− 0.4 | 1.000 | 2.5 +/− 0.3 | 0.4262 |
| A1AT + Mpred | 2.5 +/− 0.6 | 0.0369 | 1.3 +/− 0.3 | 0.0360 | 1.5 +/− 0.3 | 0.0273 |

Table 3 shows histopathology scores in a blinded study. An NMO mouse model as described above was used. Spines were collected from 4 mice per treatment cohort on Day 18 and prepared for histology. Sections from cervical, thoracic and lumbar spine regions were scored and averaged. Table 3 shows the number of inflammatory foci per section >20 cells+/−SD, p-value by two-tailed Student's t-test; demyelination scoring of hematoxylin and eosin stained section+/−SD, p-value by Mann-Whitney U test; and apoptotic cell counts+/−SD, p-value by two-tailed Student's t-test. Bold formatting indicates significant (p<0.05) difference of treatment compared to PBS (control).

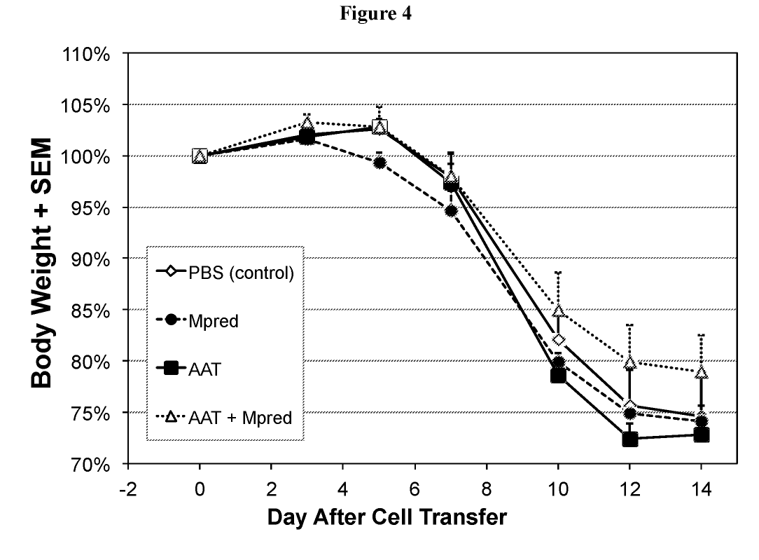

Body weight loss results from, and is a prognostic measurement of, autoimmune diseases such as NMO. A1AT+Mpred resulted in less body weight loss than either treatment alone (see FIG. 4). Ten recipient mice per cohort were used in this blinded study. The graph shows mean body weight as a percentage of the value measured at day 0+/−SEM. Interestingly, Mpred treatment alone appeared to cause early weight loss from Day 5 to Day 7. While not being bound to theory, this suggests the possibility that weight loss is a side effect of the steroid rather than NMO disease. At later times, the Mpred cohort weight loss was similar to PBS (control) even though Mpred had a lower disease score. Thus, Mpred toxicity side effects may be ameliorated by the AAT+Mpred combination cohort.

### Example 4—A1AT+Methylprednisolone Reduces Autoimmune Cytokine Profile

Ex vivo cytokine measurements from spleen cells of Th17-polarized cell transplanted mice restimulated with MOG peptide were conducted to survey effector and memory recall responses of the immune cell compartment, primarily from T cells. Spleens were collected from 6 mice per treatment cohort on Day 18. Single cell suspensions were prepared and cultured with the indicated amounts of MOG35-55 peptide. Supernatants were collected after 3 days of culture and IL-17A, IFN-γ, IL-6, or IL-2 measured by cytokine bead assay (BD Biosciences, San Jose, Calif., Cat. No. 560485). The results in this blinded study were expressed in cytokine pg/mL+/−SEM.

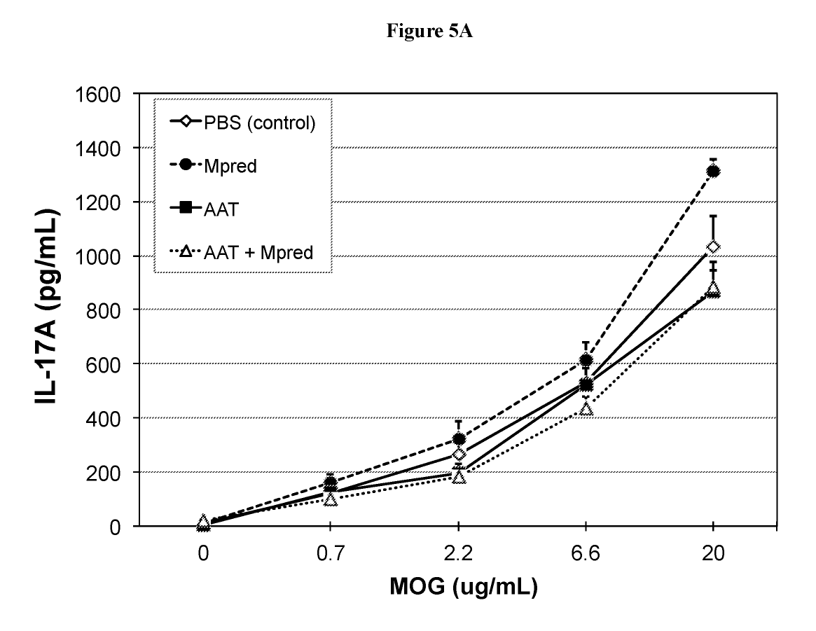

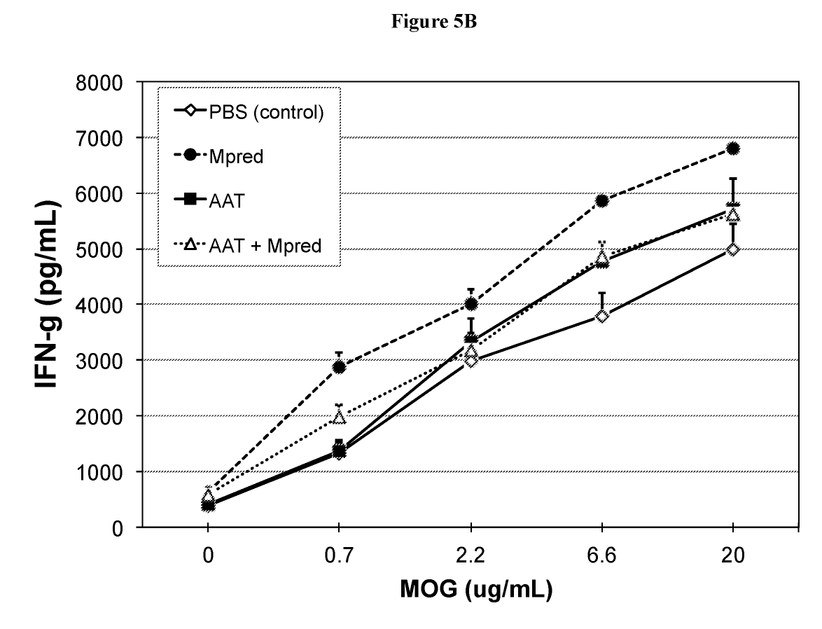

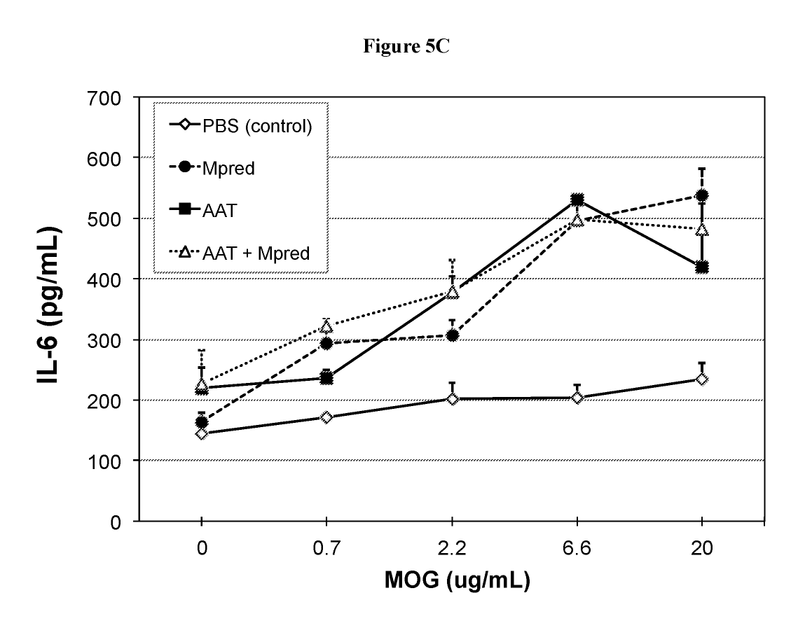

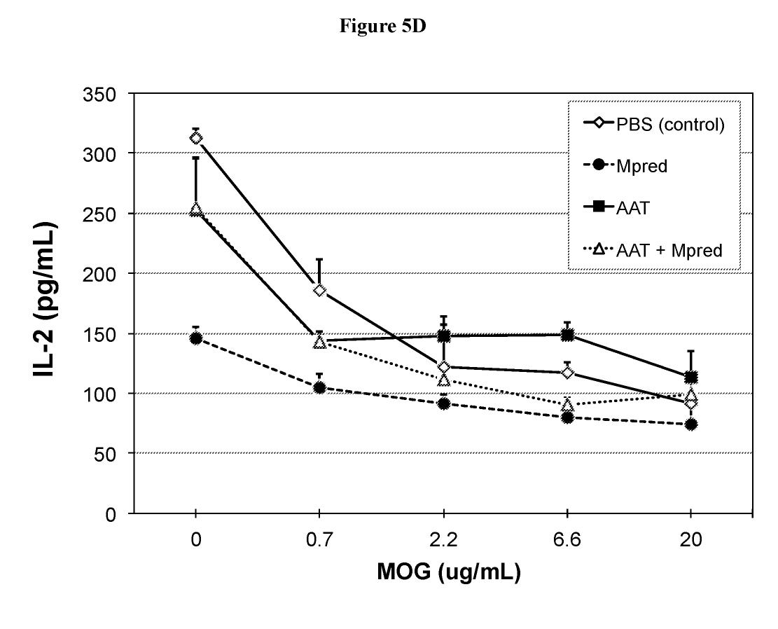

Consistent with the mouse NMO model, A1AT alone and A1AT+Mpred decreased the inflammatory cytokines IL-17A compared to PBS and Mpred alone (FIG. 5A), and decreased IFN-γ when compared to Mpred alone (FIG. 5B). All treatments resulted in increased IL-6 compared to PBS (FIG. 5C). IL-2 levels were higher without antigenic restimulation (0 MOG peptide) when treated with A1AT alone or A1AT+Mpred when compared to Mpred alone (FIG. 5D). IL-2 promotes T cell proliferation but is also a key cytokine for the maintenance of regulatory T cells in vivo. TNF-α levels were similar and IL-4 levels were at the borderline limit of detection in all four cohorts (data not shown). These cytokine restimulation measurements show that AAT and Mpred treatments resulted in differential immunomodulation of NMO disease that helps explain their additional therapeutic efficacy when used in combination.

Exemplary Sequences

Sequence ID No. 1
AlAT (Human) Protein
MPSSVSWGIL LLAGLCCLVP VSLAEDPQGD AAQKTDTSHH DQDHPTFNKI
TPNLAEFAFS LYRQLAHQSN STNIFFSPVS IATAFAMLSL GTKADTHDEI
LEGLNFNLTE IPEAQIHEGF QELLRTLNQP DSQLQLTTGN GLFLSEGLKL
VDKFLEDVKK LYHSEAFTVN FGDTEEAKKQ INDYVEKGTQ GKIVDLVKEL
DRDTVFALVN YIFFKGKWER PFEVKDTEEE DFHVDQVTTV KVPMMKRLGM
FNIQHCKKLS SWVLLMKYLG NATAIFFLPD EGKLQHLENE LTHDIITKFL
ENEDRRSASL HLPKLSITGT YDLKSVLGQL GITKVFSNGA DLSGVTEEAP
LKLSKAVHKA VLTIDEKGTE AAGAMFLEAI PMSIPPEVKF NKPFVFLMIE
QNTKSPLFMG KVVNPTQK
SEQ ID No. 2
AlAT (Human) cDNA
ATGCCGTCTTCTGTCTCGTGGGGCATCCTCCTGCTGGCAGGCCTGTGCTGCCT
GGTCCCTGTCTCCCTGGCTGAGGATCCCCAGGGAGATGCTGCCCAGAAGACA
GATACATCCCACCATGATCAGGATCACCCAACCTTCAACAAGATCACCCCCA
ACCTGGCTGAGTTCGCCTTCAGCCTATACCGCCAGCTGGCACACCAGTCCAAC
AGCACCAATATCTTCTTCTCCCCAGTGAGCATCGCTACAGCCTTTGCAATGCT
CTCCCTGGGGACCAAGGCTGACACTCACGATGAAATCCTGGAGGGCCTGAAT
TTCAACCTCACGGAGATTCCGGAGGCTCAGATCCATGAAGGCTTCCAGGAAC
TCCTCCGTACCCTCAACCAGCCAGACAGCCAGCTCCAGCTGACCACCGGCAA
TGGCCTGTTCCTCAGCGAGGGCCTGAAGCTAGTGGATAAGTTTTTGGAGGATG
TTAAAAAGTTGTACCACTCAGAAGCCTTCACTGTCAACTTCGGGGACACCGA
AGAGGCCAAGAAACAGATCAACGATTACGTGGAGAAGGGTACTCAAGGGAA
AATTGTGGATTTGGTCAAGGAGCTTGACAGAGACACAGTTTTTGCTCTGGTGA
ATTACATCTTCTTTAAAGGCAAATGGGAGAGACCCTTTGAAGTCAAGGACAC
CGAGGAAGAGGACTTCCACGTGGACCAGGTGACCACCGTGAAGGTGCCTATG
ATGAAGCGTTTAGGCATGTTTAACATCCAGCACTGTAAGAAGCTGTCCAGCTG
GGTGCTGCTGATGAAATACCTGGGCAATGCCACCGCCATCTTCTTCCTGCCTG
ATGAGGGGAAACTACAGCACCTGGAAAATGAACTCACCCACGATATCATCAC
CAAGTTCCTGGAAAATGAAGACAGAAGGTCTGCCAGCTTACATTTACCCAAA
CTGTCCATTACTGGAACCTATGATCTGAAGAGCGTCCTGGGTCAACTGGGCAT
CACTAAGGTCTTCAGCAATGGGGCTGACCTCTCCGGGGTCACAGAGGAGGCA
CCCCTGAAGCTCTCCAAGGCCGTGCATAAGGCTGTGCTGACCATCGACGAGA
AAGGGACTGAAGCTGCTGGGGCCATGTTTTTAGAGGCCATACCCATGTCTATC
CCCCCCGAGGTCAAGTTCAACAAACCCTTTGTCTTCTTAATGATTGAACAAAA
TACCAAGTCTCCCCTCTTCATGGGAAAAGTGGTGAATCCCACCCAAAAATAA
SEQ ID No. 3
CrmA (Cowpox virus) Protein
MDIFREIASSMKGENVFISPPSISSVLTILYYGANGSTAEQLSKYVEKEADKNKDDI
SFKSMNKVYGRYSAVFKDSFLRKIGDNFQTVDFTDCRTVDAINKCVDIFTEGKIN
PLLDEPLSPDTCLLAISAVYFKAKWLMPFEKEFTSDYPFYVSPTEMVDVSMMSM
YGEAFNHASVKESFGNFSIIELPYVGDTSMVVILPDNIDGLESIEQNLTDTNFKKW
CDSMDAMFIDVHIPKFKVTGSYNLVDALVKLGLTEVFGSTGDYSNMCNSDVSVD
AMIHKTYIDVNEEYTEAAAATCALVADCASTVTNEFCADHPFIYVIRHVDGKILF
VGRYCSPTTN
SEQ ID No. 4
CrmA (Cowpox virus) cDNA
ATGGATATCTTCAGGG AAATCGCATC TTCTATGAAA GGAGAGAATG
TATTCATTTC TCCACCGTCAATCTCGTCAG TATTGACAAT ACTGTATTAT
GGAGCTAATG GATCCACTGC TGAACAGCTATCAAAATATG TAGAAAAGGA
GGCGGACAAG AATAAGGATG ATATCTCATT CAAGTCCATGAATAAAGTAT
ATGGGCGATA TTCTGCAGTG TTTAAAGATT CCTTTTTGAG AAAAATTGGA
GATAATTTCC AAACTGTTGA CTTCACTGAT TGTCGCACTG TAGATGCGAT
CAACAAGTGTGTTGATATCT TCACTGAGGG GAAAATTAAT CCACTATTGG
ATGAACCATT GTCTCCAGATACCTGTCTCC TAGCAATTAG TGCCGTATAC
TTTAAAGCAA AATGGTTGAT GCCATTTGAAAAGGAATTTA CCAGTGATTA
TCCCTTTTAC GTATCTCCAA CGGAAATGGT AGATGTAAGTATGATGTCTA
TGTACGGCGA GGCATTTAAT CACGCATCTG TAAAAGAATC ATTCGGCAAC
TTTTCAATCA TAGAACTGCC ATATGTTGGA GATACTAGTA TGGTGGTAAT
TCTTCCAGACAATATTGATG GACTAGAATC CATAGAACAA AATCTAACAG
ATACAAATTT TAAGAAATGGTGTGACTCTA TGGATGCTAT GTTTATCGAT
GTGCACATTC CCAAGTTTAA GGTAACAGGCTCGTATAATC TGGTGGATGC
GCTAGTAAAG TTGGGACTGA CAGAGGTGTT CGGTTCAACTGGAGATTATA
GCAATATGTG TAATTCAGAT GTGAGTGTCG ACGCTATGAT CCACAAAACG
TATATAGATG TCAATGAAGA GTATACAGAA GCAGCTGCAG CAACTTGTGC
GCTGGTGGCAGACTGTGCAT CAACAGTTAC AAATGAGTTC TGTGCAGATC
ATCCGTTCAT CTATGTGATTAGGCATGTCG ATGGCAAAAT TCTTTTCGTT
GGTAGATATT GCTCTCCAAC AACTAATTAA
SEQ ID No. 5
Serpin1 (Arabidopsis) Protein
MDVRESISLQ NQVSMNLAKH VITTVSQNSN VIFSPASINV VLSIIAAGSA
GATKDQILSFLKFSSTDQLN SFSSEIVSAV LADGSANGGP KLSVANGAWI
DKSLSFKPSF KQLLEDSYKAASNQADFQSK AVEVIAEVNS WAEKETNGLI
TEVLPEGSAD SMTKLIFANA LYFKGTWNEKFDESLTQEGE FHLLDGNKVT
APFMTSKKKQ YVSAYDGFKV LGLPYLQGQD KRQFSMYFYLPDANNGLSDL
LDKIVSTPGF LDNHIPRRQV KVREFKIPKF KFSFGFDASN VLKGLGLTSP
FSGEEGLTEM VESPEMGKNL CVSNIFHKAC IEVNEEGTEA AAASAGVIKL
RGLLMEEDEIDFVADHPFLL VVTENITGVV LFIGQVVDPL H
SEQ ID No. 6
Serpin1 (Arabidopsis) DNA
CGTCTTCTCC TAAACCCAGC AAATTCGTTT ACCAGTCATC ACCACCACAA
CCTCCGGCGA AAATGGACGT GCGTGAATCA ATCTCACTGC AAAACCAAGT
CTCCATGAAT CTCGCAAAAC ACGTAATCAC CACCGTCTCT CAAAACTCCA
ACGTCATCTT CTCACCGGCT TCAATCAACG TCGTACTCAG TATAATCGCC
GCTGGATCCG CCGGCGCTAC CAAAGATCAG ATCCTCTCGT TTCTCAAATT
CTCTTCCACT GATCAACTTA ATTCATTCTC TTCCGAAATC GTCTCCGCTG
TTCTCGCTGA CGGTAGTGCT AACGGTGGTC CTAAGCTCTC GGTGGCTAAT
GGCGCCTGGA TCGATAAGTC TCTCTCCTTT AAACCTTCCT TTAAACAGCT
CTTGGAAGAT TCGTATAAAG CTGCTTCGAA TCAAGCTGAT TTTCAATCGA
AGGCTGTGGA GGTGATTGCT GAAGTGAATT CATGGGCTGA AAAGGAGACA
AATGGTCTCA TCACTGAGGT TCTTCCAGAA GGATCAGCTG ATAGTATGAC
CAAACTGATA TTTGCAAATG CATTGTACTT CAAGGGAACA TGGAACGAGA
AATTCGATGA GTCGTTAACA CAAGAAGGCG AGTTTCACCT TCTTGACGGT
AACAAAGTGA CTGCACCATT CATGACCAGC AAGAAGAAAC AATACGTAAG
TGCTTACGAT GGTTTCAAAG TTTTGGGACT TCCTTACTTA CAAGGACAGG
ATAAGCGACA ATTCTCCATG TACTTTTATC TTCCCGATGC AAACAACGGA
CTGTCTGATC TTCTGGACAA AATAGTTTCC ACTCCTGGGT TCTTAGACAA
CCACATCCCA CGCAGACAAG TTAAAGTCCG CGAATTCAAG ATTCCAAAGT
TTAAATTCTC TTTCGGGTTC GATGCTTCAA ATGTTTTAAA AGGATTGGGA
CTGACTTCGC CTTTCAGCGG TGAAGAAGGT TTAACTGAGA TGGTTGAATC
TCCTGAGATG GGGAAGAATC TATGCGTATC GAACATTTTC CATAAAGCGT
GTATCGAAGT GAATGAAGAA GGAACAGAAG CTGCAGCTGC ATCAGCTGGA
GTTATAAAGC TAAGAGGATT GCTTATGGAG GAAGATGAAA TAGATTTTGT
TGCAGACCAT CCGTTTCTAT TGGTGGTCAC AGAGAACATA ACAGGAGTGG
TTCTGTTCAT TGGCCAAGTT GTTGATCCGT TGCATTAATC TAAAGCTAAT
GTGGAAGTTT TTGGTTTTAC TTAAAATAAA TGAGTCATTG GTTTTGAGGA
CTCATCTTTA TGTAACATCC TTTGTCTTGA CTCTTTGATG TGTGTAAGAA
TAATAGTGAT ACATAACAGC TTTTCTTCTG TATTTGGATC ACATGTACTG
AACTGATAGA CATACATACA TGTGATGCAT CTTAATGATT CACTGT
SEQ ID No. 7
SerpinB9 (Human) protein
METLSNASGTFAIRLLKILCQDNPSHNVFCSPVSISSALAMVLLGAKGNTATQMA
QALSLNTEEDIHRAFQSLLTEVNKAGTQYLLRTANRLFGEKTCQFLSTFKESCLQF
YHAELKELSFIRAAEESRKHINTWVSKKTEGKIEELLPGSSIDAETRLVLVNAIYFK
GKWNEPFDETYTREMPFKINQEEQRPVQMMYQEATFKLAHVGEVRAQLLELPY
ARKELSLLVLLPDDGVELSTVEKSLTFEKLTAWTKPDCMKSTEVEVLLPKFKLQE
DYDMESVLRHLGIVDAFQQGKADLSAMSAERDLCLSKFVHKSFVEVNEEGTEAA
AASSCFVVAECCMESGPRFCADHPFLFFIRHNRANSILFCGRFSSP
SEQ ID No. 8
SerpinB9 (Human) cDNA
ATGGAAACTC TTTCTAATGC AAGTGGTACT TTTGCCATAC GCCTTTTAAA
GATACTGTGTCAAGATAACC CTTCGCACAA CGTGTTCTGT TCTCCTGTGA
GCATCTCCTC TGCCCTGGCCATGGTTCTCC TAGGGGCAAA GGGAAACACC
GCAACCCAGA TGGCCCAGGC ACTGTCTTTAAACACAGAGG AAGACATTCA
TCGGGCTTTC CAGTCGCTTC TCACTGAAGT GAACAAGGCTGGCACACAGT
ACCTGCTGAG AACGGCCAAC AGGCTCTTTG GAGAGAAAAC TTGTCAGTTC
CTCTCAACGT TTAAGGAATC CTGTCTTCAA TTCTACCATG CTGAGCTGAA
GGAGCTTTCCTTTATCAGAG CTGCAGAAGA GTCCAGGAAA CACATCAACA
CCTGGGTCTC AAAAAAGACCGAAGGTAAAA TTGAAGAGTT GTTGCCGGGT
AGCTCAATTG ATGCAGAAAC CAGGCTGGTTCTTGTCAATG CCATCTACTT
CAAAGGAAAG TGGAATGAAC CGTTTGACGA AACATACACAAGGGAAATGC
CCTTTAAAAT AAACCAGGAG GAGCAAAGGC CAGTGCAGAT GATGTATCAG
GAGGCCACGT TTAAGCTCGC CCACGTGGGC GAGGTGCGCG CGCAGCTGCT
GGAGCTGCCCTACGCCAGGA AGGAGCTGAG CCTGCTGGTG CTGCTGCCTG
ACGACGGCGT GGAGCTCAGCAAGAGTACTG AGGTTGAAGT TCTCCTTCCA
AAATTTAAAC TACAAGAGGA TTATGACATGGAATCTGTGC TTCGGCATTT
GGGAATTGTT GATGCCTTCC AACAGGGCAA GGCTGACTTGTCGGCAATGT
CAGCGGAGAG AGACCTGTGT CTGTCCAAGT TCGTGCACAA GAGTTTTGTG
GAGGTGAATG AAGAAGGCAC CGAGGCAGCG GCAGCGTCGA GCTGCTTTGT
AGTTGCAGAGTGCTGCATGG AATCTGGCCC CAGGTTCTGT GCTGACCACC
CTTTCCTTTT CTTCATCAGGCACAACAGAG CCAACAGCAT TCTGTTCTGT
GGCAGGTTCT CATCGCCA
SEQ ID No. 9
SerpinA3 (Human) Protein
MERMLPLLTL GLLAAGFCPA VLCHPNSPLD EENLTQENQD RGTHVDLGLA
SANVDFAFSLYKQSPRWSIR LCLMYLRRAQ KHLLPQQSKS PSFLH
SEQ ID No. 10
SerpinA3 (Human) Protein Precursor
MERMLPLLALGLLAAGFCPAVLCHPNSPLDEENLTQENQDRGTHVDLGLASANV
DFAFSLYKQLVLKAPDKNVIFSPLSISTALAFLSLGAHNTTLTEILKGLKFNLTETS
EAEIHQSFQHLLRTLNQSSDELQLSMGNAMFVKEQLSLLDRFTEDAKRLYGSEAF
ATDFQDSAAAKKLINDYVKNGTRGKITDLIKDLDSQTMMVLVNYIFFKAKWEMP
FDPQDTHQSRFYLSKKKWVMVPMMSLHHLTIPYFRDEELSCTVVELKYTGNASA
LFILPDQDKMEEVEAMLLPETLKRWRDSLEFREIGELYLPKFSISRDYNLNDILLQ
LGIEEAFTSKADLSGITGARNLAVSQVVHKAVLDVFEEGTEASAATAVKITLLSA
LVETRTIVRFNRPFLMIIVPTDTQNIFFMSKVTNPKQA
SEQ ID No. 11
SerpinA3 (Human) cDNA precursor
ATGGAGAGAAT GTTACCTCTC CTGGCTCTGG GGCTCTTGGC GGCTGGGTTC
TGCCCTGCTG TCCTCTGCCA CCCTAACAGC CCACTTGACG AGGAGAATCT
GACCCAGGAG AACCAAGACC GAGGGACACA CGTGGACCTC GGATTAGCCT
CCGCCAACGT GGACTTCGCT TTCAGCCTGT ACAAGCAGTT AGTCCTGAAG
GCCCCTGATA AGAATGTCAT CTTCTCCCCA CTGAGCATCT CCACCGCCTT
GGCCTTCCTG TCTCTGGGGG CCCATAATAC CACCCTGACA GAGATTCTCA
AAGGCCTCAA GTTCAACCTC ACGGAGACTT CTGAGGCAGA AATTCACCAG
AGCTTCCAGC ACCTCCTGCG CACCCTCAAT CAGTCCAGCG ATGAGCTGCA
GCTGAGTATG GGAAATGCCA TGTTTGTCAA AGAGCAACTC AGTCTGCTGG
ACAGGTTCAC GGAGGATGCC AAGAGGCTGT ATGGCTCCGA GGCCTTTGCC
ACTGACTTTC AGGACTCAGC TGCAGCTAAG AAGCTCATCA ACGACTACGT
GAAGAATGGA ACTAGGGGGA AAATCACAGA TCTGATCAAG GACCTTGACT
CGCAGACAAT GATGGTCCTG GTGAATTACA TCTTCTTTAA AGCCAAATGG
GAGATGCCCT TTGACCCCCA AGATACTCAT CAGTCAAGGT TCTACTTGAG
CAAGAAAAAG TGGGTAATGG TGCCCATGAT GAGTTTGCAT CACCTGACTA
TACCTTACTT CCGGGACGAG GAGCTGTCCT GCACCGTGGT GGAGCTGAAG
TACACAGGCA ATGCCAGCGC ACTCTTCATC CTCCCTGATC AAGACAAGAT
GGAGGAAGTG GAAGCCATGC TGCTCCCAGA GACCCTGAAG CGGTGGAGAG
ACTCTCTGGA GTTCAGAGAG ATAGGTGAGC TCTACCTGCC AAAGTTTTCC
ATCTCGAGGG ACTATAACCT GAACGACATA CTTCTCCAGC TGGGCATTGA
GGAAGCCTTC ACCAGCAAGG CTGACCTGTC AGGGATCACA GGGGCCAGGA
ACCTAGCAGT CTCCCAGGTG GTCCATAAGG CTGTGCTTGA TGTATTTGAG
GAGGGCACAG AAGCATCTGC TGCCACAGCA GTCAAAATCA CCCTCCTTTC
TGCATTAGTG GAGACAAGGA CCATTGTGCG TTTCAACAGG CCCTTCCTGA
TGATCATTGT CCCTACAGAC ACCCAGAACA TCTTCTTCAT GAGCAAAGTC
ACCAATCCCA AGCAAGCCTA G
SEQ ID No. 12
SerpinA4 transcript variant 3 (Human) Protein
MHLIDYLLLLLVGLLALSHGQLHVEHDGESCSNSSHQQILETGEGSPSLKIAPANA
DFAFRFYYLIASETPGKNIFFSPLSISAAYAMLSLGACSHSRSQILEGLGFNLTELSE
SDVHRGFQHLLHTLNLPGHGLETRVGSALFLSHNLKFLAKFLNDTMAVYEAKLF
HTNFYDTVGTIQLINDHVKKETRGKIVDLVSELKKDVLMVLVNYIYFKALWEKP
FISSRTTPKDFYVDENTTVRVPMMLQDQEHHWYLHDRYLPCSVLRMDYKGDAT
VFFILPNQGKMREIEEVLTPEMLMRWNNLLRKRNFYKKLELHLPKFSISGSYVLD
QILPRLGFTDLFSKWADLSGITKQQKLEASKSFHKATLDVDEAGTEAAAATSFAI
KFFSAQTNRHILRFNRPFLVVIFSTSTQSVLFLGKVVDPTKP
SEQ ID No. 13
SerpinA4 transcript variant 3 (Human) cDNA
ATGCATCT TATCGACTAC CTGCTCCTCC TGCTGGTTGG ACTACTGGCC
CTTTCTCATG GCCAGCTGCA CGTTGAGCAT GATGGTGAGA GTTGCAGTAA
CAGCTCCCAC CAGCAGATTC TGGAGACAGG TGAGGGCTCC CCCAGCCTCA
AGATAGCCCC TGCCAATGCT GACTTTGCCT TCCGCTTCTA CTACCTGATC
GCTTCGGAGA CCCCGGGGAA GAACATCTTT TTCTCCCCGC TGAGCATCTC
GGCGGCCTAC GCCATGCTTT CCCTGGGGGC CTGCTCACAC AGCCGCAGCC
AGATCCTTGA GGGCCTGGGC TTCAACCTCA CCGAGCTGTC TGAGTCCGAT
GTCCATAGGG GCTTCCAGCA CCTCCTGCAC ACTCTCAACC TCCCCGGCCA
TGGGCTGGAA ACACGCGTGG GCAGTGCTCT GTTCCTGAGC CACAACCTGA
AGTTCCTTGC AAAATTCCTG AATGACACCA TGGCCGTCTA TGAGGCTAAA
CTCTTCCACA CCAACTTCTA CGACACTGTG GGCACAATCC AGCTTATCAA
CGACCACGTC AAGAAGGAAA CTCGAGGGAA GATTGTGGAT TTGGTCAGTG
AGCTCAAGAA GGACGTCTTG ATGGTGCTGG TGAATTACAT TTACTTCAAA
GCCCTGTGGG AGAAACCATT CATTTCCTCA AGGACCACTC CCAAAGACTT
CTATGTTGAT GAGAACACAA CAGTCCGGGT GCCCATGATG CTGCAGGACC
AGGAGCATCA CTGGTATCTT CATGACAGAT ACTTGCCCTG CTCGGTGCTA
CGGATGGATT ACAAAGGAGA CGCAACCGTG TTTTTCATTC TCCCTAACCA
AGGCAAAATG AGGGAGATTG AAGAGGTTCT GACTCCAGAG ATGCTAATGA
GGTGGAACAA CTTGTTGCGG AAGAGGAATT TTTACAAGAA GCTAGAGTTG
CATCTTCCCA AGTTCTCCAT TTCTGGCTCC TATGTATTAG ATCAGATTTT
GCCCAGGCTG GGCTTCACGG ATCTGTTCTC CAAGTGGGCT GACTTATCCG
GCATCACCAA ACAGCAAAAA CTGGAGGCAT CCAAAAGTTT CCACAAGGCC
ACCTTGGACG TGGATGAGGC TGGCACCGAG GCTGCAGCAG CCACCAGCTT
CGCGATCAAA TTCTTCTCTG CCCAGACCAA TCGCCACATC CTGCGATTCA
ACCGGCCCTT CCTTGTGGTG ATCTTTTCCA CCAGCACCCA GAGTGTCCTC
TTTCTGGGCA AGGTCGTCGA CCCCACGAAA CCATAG
SEQ ID No. 14
SerpinA4 transcript variant 2 (Human) Protein
MHLIDYLLLLLVGLLALSHGQLHVEHDGESCSNSSHQQILETGEGSPSLKIAPANA
DFAFRFYYLIASETPGKNIFFSPLSISAAYAMLSLGACSHSRSQILEGLGFNLTELSE
SDVHRGFQHLLHTLNLPGHGLETRVGSALFLSHNLKFLAKFLNDTMAVYEAKLF
HTNFYDTVGTIQLINDHVKKETRGKIVDLVSELKKDVLMVLVNYIYFKALWEKP
FISSRTTPKDFYVDENTTVRVPMMLQDQEHHWYLHDRYLPCSVLRMDYKGDAT
VFFILPNQGKMREIEEVLTPEMLMRWNNLLRKRNFYKKLELHLPKFSISGSYVLD
QILPRLGFTDLFSKWADLSGITKQQKLEASKSFHKATLDVDEAGTEAAAATSFAI
KFFSAQTNRHILRFNRPFLVVIFSTSTQSVLFLGKVVDPTKP
SEQ ID No. 15
SerpinA4 transcript variant 2 (Human) cDNA
ATGCATCT TATCGACTAC CTGCTCCTCC TGCTGGTTGG ACTACTGGCC
CTTTCTCATG GCCAGCTGCA CGTTGAGCAT GATGGTGAGA GTTGCAGTAA
CAGCTCCCAC CAGCAGATTC TGGAGACAGG TGAGGGCTCC CCCAGCCTCA
AGATAGCCCC TGCCAATGCT GACTTTGCCT TCCGCTTCTA CTACCTGATC
GCTTCGGAGA CCCCGGGGAA GAACATCTTT TTCTCCCCGC TGAGCATCTC
GGCGGCCTAC GCCATGCTTT CCCTGGGGGC CTGCTCACAC AGCCGCAGCC
AGATCCTTGA GGGCCTGGGC TTCAACCTCA CCGAGCTGTC TGAGTCCGAT
GTCCATAGGG GCTTCCAGCA CCTCCTGCAC ACTCTCAACC TCCCCGGCCA
TGGGCTGGAA ACACGCGTGG GCAGTGCTCT GTTCCTGAGC CACAACCTGA
AGTTCCTTGC AAAATTCCTG AATGACACCA TGGCCGTCTA TGAGGCTAAA
CTCTTCCACA CCAACTTCTA CGACACTGTG GGCACAATCC AGCTTATCAA
CGACCACGTC AAGAAGGAAA CTCGAGGGAA GATTGTGGAT TTGGTCAGTG
AGCTCAAGAA GGACGTCTTG ATGGTGCTGG TGAATTACAT TTACTTCAAA
GCCCTGTGGG AGAAACCATT CATTTCCTCA AGGACCACTC CCAAAGACTT
CTATGTTGAT GAGAACACAA CAGTCCGGGT GCCCATGATG CTGCAGGACC
AGGAGCATCA CTGGTATCTT CATGACAGAT ACTTGCCCTG CTCGGTGCTA
CGGATGGATT ACAAAGGAGA CGCAACCGTG TTTTTCATTC TCCCTAACCA
AGGCAAAATG AGGGAGATTG AAGAGGTTCT GACTCCAGAG ATGCTAATGA
GGTGGAACAA CTTGTTGCGG AAGAGGAATT TTTACAAGAA GCTAGAGTTG
CATCTTCCCA AGTTCTCCAT TTCTGGCTCC TATGTATTAG ATCAGATTTT
GCCCAGGCTG GGCTTCACGG ATCTGTTCTC CAAGTGGGCT GACTTATCCG
GCATCACCAA ACAGCAAAAA CTGGAGGCAT CCAAAAGTTT CCACAAGGCC
ACCTTGGACG TGGATGAGGC TGGCACCGAG GCTGCAGCAG CCACCAGCTT
CGCGATCAAA TTCTTCTCTG CCCAGACCAA TCGCCACATC CTGCGATTCA
ACCGGCCCTT CCTTGTGGTG ATCTTTTCCA CCAGCACCCA GAGTGTCCTC
TTTCTGGGCA AGGTCGTCGA CCCCACGAAA CCATAG
SEQ ID No. 16
SerpinA4 transcript variant 1 (Human) Protein
MPGDPEKPPPGTHSWYRAALTEGQGLLAANPGLRVQRMHLIDYLLLLLVGLLAL
SHGQLHVEHDGESCSNSSHQQILETGEGSPSLKIAPANADFAFRFYYLIASETPGK
NIFFSPLSISAAYAMLSLGACSHSRSQILEGLGFNLTELSESDVHRGFQHLLHTLNL
PGHGLETRVGSALFLSHNLKFLAKFLNDTMAVYEAKLFHTNFYDTVGTIQLINDH
VKKETRGKIVDLVSELKKDVLMVLVNYIYFKALWEKPFISSRTTPKDFYVDENT
TVRVPMMLQDQEHHWYLHDRYLPCSVLRMDYKGDATVFFILPNQGKMREIEEV
LTPEMLMRWNNLLRKRNFYKKLELHLPKFSISGSYVLDQILPRLGFTDLFSKWAD
LSGITKQQKLEASKSFHKATLDVDEAGTEAAAATSFAIKFFSAQTNRHILRFNRPF
LVVIFSTSTQSVLFLGKVVDPTKP
SEQ ID No. 17
SerpinA4 transcript variant 1 (Human) cDNA
ATGC CCGGAGACCC AGAAAAGCCT CCCCCAGGGA CACACAGCTG
GTACAGGGCG GCACTGACTG AGGGCCAAGG TCTTCTGGCT GCCAATCCAG
GCCTGAGAGT GCAGAGGATG CATCTTATCG ACTACCTGCT CCTCCTGCTG
GTTGGACTAC TGGCCCTTTC TCATGGCCAG CTGCACGTTG AGCATGATGG
TGAGAGTTGC AGTAACAGCT CCCACCAGCA GATTCTGGAG ACAGGTGAGG
GCTCCCCCAG CCTCAAGATA GCCCCTGCCA ATGCTGACTT TGCCTTCCGC
TTCTACTACC TGATCGCTTC GGAGACCCCG GGGAAGAACA TCTTTTTCTC
CCCGCTGAGC ATCTCGGCGGCCTACGCCAT GCTTTCCCTG GGGGCCTGCT
CACACAGCCG CAGCCAGATC CTTGAGGGCCTGGGCTTCAA CCTCACCGAG
CTGTCTGAGT CCGATGTCCA TAGGGGCTTC CAGCACCTCCTGCACACTCT
CAACCTCCCC GGCCATGGGC TGGAAACACG CGTGGGCAGT GCTCTGTTCC
TGAGCCACAA CCTGAAGTTC CTTGCAAAAT TCCTGAATGA CACCATGGCC
GTCTATGAGGCTAAACTCTT CCACACCAAC TTCTACGACA CTGTGGGCAC
AATCCAGCTT ATCAACGACCACGTCAAGAA GGAAACTCGA GGGAAGATTG
TGGATTTGGT CAGTGAGCTC AAGAAGGACGTCTTGATGGT GCTGGTGAAT
TACATTTACT TCAAAGCCCT GTGGGAGAAA CCATTCATTTCCTCAAGGAC
CACTCCCAAA GACTTCTATG TTGATGAGAA CACAACAGTC CGGGTGCCCA
TGATGCTGCA GGACCAGGAG CATCACTGGT ATCTTCATGA CAGATACTTG
CCCTGCTCGGTGCTACGGAT GGATTACAAA GGAGACGCAA CCGTGTTTTT
CATTCTCCCT AACCAAGGCAAAATGAGGGA GATTGAAGAG GTTCTGACTC
CAGAGATGCT AATGAGGTGG AACAACTTGTTGCGGAAGAG GAATTTTTAC
AAGAAGCTAG AGTTGCATCT TCCCAAGTTC TCCATTTCTGGCTCCTATGT
ATTAGATCAG ATTTTGCCCA GGCTGGGCTT CACGGATCTG TTCTCCAAGT
GGGCTGACTT ATCCGGCATC ACCAAACAGC AAAAACTGGA GGCATCCAAA
AGTTTCCACAAGGCCACCTT GGACGTGGAT GAGGCTGGCA CCGAGGCTGC
AGCAGCCACC AGCTTCGCGATCAAATTCTT CTCTGCCCAG ACCAATCGCC
ACATCCTGCG ATTCAACCGG CCCTTCCTTGTGGTGATCTT TTCCACCAGC
ACCCAGAGTG TCCTCTTTCT GGGCAAGGTC GTCGACCCCACGAAACCATA G
SEQ ID No. 18
SerpinA5 (Human) Protein
MQLFLLLCLVLLSPQGASLHRHHPREMKKRVEDLHVGATVAPSSRRDFTFDLYR
ALASAAPSQSIFFSPVSISMSLAMLSLGAGSSTKMQILEGLGLNLQKSSEKELHRG
FQQLLQELNQPRDGFQLSLGNALFTDLVVDLQDTFVSAMKTLYLADTFPTNFRD
SAGAMKQINDYVAKQTKGKIVDLLKNLDSNAVVIMVNYIFFKAKWETSFNHKG
TQEQDFYVTSETVVRVPMMSREDQYHYLLDRNLSCRVVGVPYQGNATALFILPS
EGKMQQVENGLSEKTLRKWLKMFKKRQLELYLPKFSIEGSYQLEKVLPSLGISNV
FTSHADLSGISNHSNIQVSEMVHKAVVEVDESGTRAAAATGTIFTFRSARLNSQR
LVFNRPFLMFIVDNNILFLGKVNRP
SEQ ID No. 19
SerpinA5 (Human) cDNA
ATGCAGCTCTTCCTC CTCTTGTGCC TGGTGCTTCT CAGCCCTCAG
GGGGCCTCCC TTCACCGCCACCACCCCCGG GAGATGAAGA AGAGAGTCGA
GGACCTCCAT GTAGGTGCCA CGGTGGCCCCCAGCAGCAGA AGGGACTTTA
CCTTTGACCT CTACAGGGCC TTGGCTTCCG CTGCCCCCAGCCAGAGCATC
TTCTTCTCCC CTGTGAGCAT CTCCATGAGC CTGGCCATGC TCTCCCTGGG
GGCTGGGTCC AGCACAAAGA TGCAGATCCT GGAGGGCCTG GGCCTCAACC
TCCAGAAAAGCTCAGAGAAG GAGCTGCACA GAGGCTTTCA GCAGCTCCTT
CAGGAACTCA ACCAGCCCAGAGATGGCTTC CAGCTGAGCC TCGGCAATGC
CCTTTTCACC GACCTGGTGG TAGACCTGCAGGACACCTTC GTAAGTGCCA
TGAAGACGCT GTACCTGGCA GACACTTTCC CTACCAACTTTAGGGACTCT
GCAGGGGCCA TGAAGCAGAT CAATGATTAT GTGGCAAAGC AAACGAAGGG
CAAGATTGTG GACTTGCTTA AGAACCTCGA TAGCAATGCG GTCGTGATCA
TGGTGAATTACATCTTCTTT AAAGCTAAGT GGGAGACAAG CTTCAACCAC
AAAGGCACCC AAGAGCAAGACTTCTACGTG ACCTCGGAGA CTGTGGTGCG
GGTACCCATG ATGAGCCGCG AGGATCAGTATCACTACCTC CTGGACCGGA
ACCTCTCCTG CAGGGTGGTG GGGGTCCCCT ACCAAGGCAATGCCACGGCT
TTGTTCATTC TCCCCAGTGA GGGAAAGATG CAGCAGGTGG AGAATGGACT
GAGTGAGAAA ACGCTGAGGA AGTGGCTTAA GATGTTCAAA AAGAGGCAGC
TCGAGCTTTACCTTCCCAAA TTCTCCATTG AGGGCTCCTA TCAGCTGGAG
AAAGTCCTCC CCAGTCTGGGGATCAGTAAC GTCTTCACCT CCCATGCTGA
TCTGTCCGGC ATCAGCAACC ACTCAAATATCCAGGTGTCT GAGATGGTGC
ACAAAGCTGT GGTGGAGGTG GACGAGTCGG GAACCAGAGCAGCGGCAGCC
ACGGGGACAA TATTCACTTT CAGGTCGGCC CGCCTGAACT CTCAGAGGCT
AGTGTTCAAC AGGCCCTTTC TGATGTTCAT TGTGGATAAC AACATCCTCT
TCCTTGGCAAAGTGAACCGC CCCTGA
SEQ ID No. 20
SerpinA9 transcript variant 4 (Human) Protein
MGSALFVKKELQLQANFLGNVKRLYEAEVFSTDFSNPSIAQARINSHVKKKTQG
KVVDIIQGLDLLTAMVLVNHIFFKAKWEKPFHPEYTRKNFPFLVGEQVTVHVPM
MHQKEQFAFGVDTELNCFVLQMDYKGDAVAFFVLPSKGKMRQLEQALSARTL
RKWSHSLQKRWIEVFIPRFSISASYNLETILPKMGIQNVFDKNADFSGIAKRDSLQ
VSKATHKAVLDVSEEGTEATAATTTKFIVRSKDGPSYFTVSFNRTFLMMITNKAT
DGILFLGKVENPTKS
SEQ ID No. 21
SerpinA9 transcript variant 4 (Human) cDNA
ATGGG AAGTGCCCTC TTCGTCAAGA AGGAGCTGCA GCTGCAGGCA
AATTTCTTGG GCAATGTCAA GAGGCTGTAT GAAGCAGAAG TCTTTTCTAC
AGATTTCTCCAACCCCTCCA TTGCCCAGGC GAGGATCAAC AGCCATGTGA
AAAAGAAGAC CCAAGGGAAGGTTGTAGACA TAATCCAAGG CCTTGACCTT
CTGACGGCCA TGGTTCTGGT GAACCACATTTTCTTTAAAG CCAAGTGGGA
GAAGCCCTTT CACCCTGAAT ATACAAGAAA GAACTTCCCATTCCTGGTGG
GCGAGCAGGT CACTGTGCAT GTCCCCATGA TGCACCAGAA AGAGCAGTTC
GCTTTTGGGG TGGATACAGA GCTGAACTGC TTTGTGCTGC AGATGGATTA
CAAGGGAGATGCCGTGGCCT TCTTTGTCCT CCCTAGCAAG GGCAAGATGA
GGCAACTGGA ACAGGCCTTGTCAGCCAGAA CACTGAGAAA GTGGAGCCAC
TCACTCCAGA AAAGGTGGAT AGAGGTGTTCATCCCCAGAT TTTCCATTTC
TGCCTCCTAC AATCTGGAAA CCATCCTCCC GAAGATGGGCATCCAAAATG
TCTTTGACAA AAATGCTGAT TTTTCTGGAA TTGCAAAGAG AGACTCCCTG
CAGGTTTCTA AAGCAACCCA CAAGGCTGTG CTGGATGTCA GTGAAGAGGG
CACTGAGGCCACAGCAGCTA CCACCACCAA GTTCATAGTC CGATCGAAGG
ATGGCCCCTC TTACTTCACTGTCTCCTTCA ATAGGACCTT CCTGATGATG
ATTACAAATA AAGCCACAGA CGGTATTCTCTTTCTAGGGA AAGTGGAAAA
TCCCACTAAA TCCTAG
SEQ ID No. 22
SerpinA9 transcript variant 3 (Human) Protein
MRSAGGRGEIKVRRELQPSKQVSGLTNHARTGQEKRNLQRLVLETPSQNIFFSPV
SVSTSLAMLSLGAHSVTKTQILQGLGFNLTHTPESAIHQGFQHLVHSLTVPSKDLT
LKMGSALFVKKELQLQANFLGNVKRLYEAEVFSTDFSNPSIAQARINSHVKKKT
QGKVVDIIQGLDLLTAMVLVNHIFFKAKWEKPFHPEYTRKNFPFLVGEQVTVH
VPMMHQKEQFAFGVDTELNCFVLQMDYKGDAVAFFVLPSKGKMRQLEQALSA
RTLRKWSHSLQKRWIEVFIPRFSISASYNLETILPKMGIQNVFDKNADFSGIAKRD
SLQVSKATHKAVLDVSEEGTEATAATTTKFIVRSKDGPSYFTVSFNRTFLMMITN
KATDGILFLGKVENPTKS
SEQ ID No. 23
SerpinA9 transcript variant 3 (Human) cDNA
ATGAGATCAGCTGGAGGGAGAGGAGAGATTA AAGTGAGGAGAGAGCTACAA
CCAAGTAAGC AAGTGTCAGG GCTCACCAAC CATGCAAGGA CAGGGCAGGA
GAAGAGGAAC CTGCAAAGGC TGGTTTTGGA GACCCCGAGT CAGAACATCT
TCTTCTCCCCTGTGAGTGTC TCCACTTCCC TGGCCATGCT CTCCCTTGGG
GCCCACTCAG TCACCAAGACCCAGATTCTC CAGGGCCTGG GCTTCAACCT
CACACACACA CCAGAGTCTG CCATCCACCAGGGCTTCCAG CACCTGGTTC
ACTCACTGAC TGTTCCCAGC AAAGACCTGA CCTTGAAGATGGGAAGTGCC
CTCTTCGTCA AGAAGGAGCT GCAGCTGCAG GCAAATTTCT TGGGCAATGT
CAAGAGGCTG TATGAAGCAG AAGTCTTTTC TACAGATTTC TCCAACCCCT
CCATTGCCCAGGCGAGGATC AACAGCCATG TGAAAAAGAA GACCCAAGGG
AAGGTTGTAG ACATAATCCAAGGCCTTGAC CTTCTGACGG CCATGGTTCT
GGTGAACCAC ATTTTCTTTA AAGCCAAGTGGGAGAAGCCC TTTCACCCTG
AATATACAAG AAAGAACTTC CCATTCCTGG TGGGCGAGCAGGTCACTGTG
CATGTCCCCA TGATGCACCA GAAAGAGCAG TTCGCTTTTG GGGTGGATAC
AGAGCTGAAC TGCTTTGTGC TGCAGATGGA TTACAAGGGA GATGCCGTGG
CCTTCTTTGTCCTCCCTAGC AAGGGCAAGA TGAGGCAACT GGAACAGGCC
TTGTCAGCCA GAACACTGAGAAAGTGGAGC CACTCACTCC AGAAAAGGTG
GATAGAGGTG TTCATCCCCA GATTTTCCATTTCTGCCTCC TACAATCTGG
AAACCATCCT CCCGAAGATG GGCATCCAAA ATGTCTTTGACAAAAATGCT
GATTTTTCTG GAATTGCAAA GAGAGACTCC CTGCAGGTTT CTAAAGCAAC
CCACAAGGCT GTGCTGGATG TCAGTGAAGA GGGCACTGAG GCCACAGCAG
CTACCACCACCAAGTTCATA GTCCGATCGA AGGATGGCCC CTCTTACTTC
ACTGTCTCCT TCAATAGGACCTTCCTGATG ATGATTACAA ATAAAGCCAC
AGACGGTATT CTCTTTCTAG GGAAAGTGGAAAATCCCACT AAATCCTAG
SEQ ID No. 24
SerpinA9 transcript variant 2 (Human) Protein
MQGQGRRRGTCKDIFCSKMASYLYGVLFAVGLCAPIYCVSPANAPSAYPRPSST
KSTPASQVYSLNTDFAFRLYRRLVLETPSQNIFFSPARINSHVKKKTQGKVVDIIQ
GLDLLTAMVLVNHIFFKAKWEKPFHPEYTRKNFPFLVGEQVTVHVPMMHQKEQ
FAFGVDTELNCFVLQMDYKGDAVAFFVLPSKGKMRQLEQALSARTLRKWSHSL
QKRWIEVFIPRFSISASYNLETILPKMGIQNVFDKNADFSGIAKRDSLQVSKATHK
AVLDVSEEGTEATAATTTKFIVRSKDGPSYFTVSFNRTFLMMITNKATDGILFLGK
VENPTKS
SEQ ID No. 25
SerpinA9 transcript variant 2 (Human) cDNA
ATGCAAGGA CAGGGCAGGAGAAGAGGAAC CTGCAAAGAC ATATTTTGTT
CCAAAATGGC ATCTTACCTT TATGGAGTACTCTTTGCTGT TGGCCTCTGT
GCTCCAATCT ACTGTGTGTC CCCGGCCAAT GCCCCCAGTGCATACCCCCG
CCCTTCCTCC ACAAAGAGCA CCCCTGCCTC ACAGGTGTAT TCCCTCAACA
CCGACTTTGC CTTCCGCCTA TACCGCAGGC TGGTTTTGGA GACCCCGAGT
CAGAACATCTTCTTCTCCCC TGCGAGGATC AACAGCCATG TGAAAAAGAA
GACCCAAGGG AAGGTTGTAGACATAATCCA AGGCCTTGAC CTTCTGACGG
CCATGGTTCT GGTGAACCAC ATTTTCTTTAAAGCCAAGTG GGAGAAGCCC
TTTCACCCTG AATATACAAG AAAGAACTTC CCATTCCTGGTGGGCGAGCA
GGTCACTGTG CATGTCCCCA TGATGCACCA GAAAGAGCAG TTCGCTTTTG
GGGTGGATAC AGAGCTGAAC TGCTTTGTGC TGCAGATGGA TTACAAGGGA
GATGCCGTGGCCTTCTTTGT CCTCCCTAGC AAGGGCAAGA TGAGGCAACT
GGAACAGGCC TTGTCAGCCAGAACACTGAG AAAGTGGAGC CACTCACTCC
AGAAAAGGTG GATAGAGGTG TTCATCCCCAGATTTTCCAT TTCTGCCTCC
TACAATCTGG AAACCATCCT CCCGAAGATG GGCATCCAAAATGTCTTTGA
CAAAAATGCT GATTTTTCTG GAATTGCAAA GAGAGACTCC CTGCAGGTTT
CTAAAGCAAC CCACAAGGCT GTGCTGGATG TCAGTGAAGA GGGCACTGAG
GCCACAGCAGCTACCACCAC CAAGTTCATA GTCCGATCGA AGGATGGCCC
CTCTTACTTC ACTGTCTCCTTCAATAGGAC CTTCCTGATG ATGATTACAA
ATAAAGCCAC AGACGGTATT CTCTTTCTAGGGAAAGTGGA AAATCCCACT
AAATCCTAG
SEQ ID No. 26
SerpinA9 transcript variant 1 (Human) Protein
MQGQGRRRGTCKDIFCSKMASYLYGVLFAVGLCAPIYCVSPANAPSAYPRPSST
KSTPASQVYSLNTDFAFRLYRRLVLETPSQNIFFSPVSVSTSLAMLSLGAHSVTKT
QILQGLGFNLTHTPESAIHQGFQHLVHSLTVPSKDLTLKMGSALFVKKELQLQAN
FLGNVKRLYEAEVFSTDFSNPSIAQARINSHVKKKTQGKVVDIIQGLDLLTAMVL
VNHIFFKAKWEKPFHPEYTRKNFPFLVGEQVTVHVPMMHQKEQFAFGVDTELN
CFVLQMDYKGDAVAFFVLPSKGKMRQLEQALSARTLRKWSHSLQKRWIEVFIPR
FSISASYNLETILPKMGIQNVFDKNADFSGIAKRDSLQVSKATHKAVLDVSEEGTE
ATAATTTKFIVRSKDGPSYFTVSFNRTFLMMITNKATDGILFLGKVENPTKS
SEQ ID No. 27
SerpinA9 transcript variant 1 (Human) cDNA
ATGCAAGGA CAGGGCAGGAGAAGAGGAAC CTGCAAAGAC ATATTTTGTT
CCAAAATGGC ATCTTACCTT TATGGAGTACTCTTTGCTGT TGGCCTCTGT
GCTCCAATCT ACTGTGTGTC CCCGGCCAAT GCCCCCAGTGCATACCCCCG
CCCTTCCTCC ACAAAGAGCA CCCCTGCCTC ACAGGTGTAT TCCCTCAACA
CCGACTTTGC CTTCCGCCTA TACCGCAGGC TGGTTTTGGA GACCCCGAGT
CAGAACATCTTCTTCTCCCC TGTGAGTGTC TCCACTTCCC TGGCCATGCT
CTCCCTTGGG GCCCACTCAGTCACCAAGAC CCAGATTCTC CAGGGCCTGG
GCTTCAACCT CACACACACA CCAGAGTCTGCCATCCACCA GGGCTTCCAG
CACCTGGTTC ACTCACTGAC TGTTCCCAGC AAAGACCTGACCTTGAAGAT
GGGAAGTGCC CTCTTCGTCA AGAAGGAGCT GCAGCTGCAG GCAAATTTCT
TGGGCAATGT CAAGAGGCTG TATGAAGCAG AAGTCTTTTC TACAGATTTC
TCCAACCCCTCCATTGCCCA GGCGAGGATC AACAGCCATG TGAAAAAGAA
GACCCAAGGG AAGGTTGTAGACATAATCCA AGGCCTTGAC CTTCTGACGG
CCATGGTTCT GGTGAACCAC ATTTTCTTTAAAGCCAAGTG GGAGAAGCCC
TTTCACCCTG AATATACAAG AAAGAACTTC CCATTCCTGGTGGGCGAGCA
GGTCACTGTG CATGTCCCCA TGATGCACCA GAAAGAGCAG TTCGCTTTTG
GGGTGGATAC AGAGCTGAAC TGCTTTGTGC TGCAGATGGA TTACAAGGGA
GATGCCGTGGCCTTCTTTGT CCTCCCTAGC AAGGGCAAGA TGAGGCAACT
GGAACAGGCC TTGTCAGCCAGAACACTGAG AAAGTGGAGC CACTCACTCC
AGAAAAGGTG GATAGAGGTG TTCATCCCCAGATTTTCCAT TTCTGCCTCC
TACAATCTGG AAACCATCCT CCCGAAGATG GGCATCCAAAATGTCTTTGA
CAAAAATGCT GATTTTTCTG GAATTGCAAA GAGAGACTCC CTGCAGGTTT
CTAAAGCAAC CCACAAGGCT GTGCTGGATG TCAGTGAAGA GGGCACTGAG
GCCACAGCAGCTACCACCAC CAAGTTCATA GTCCGATCGA AGGATGGCCC
CTCTTACTTC ACTGTCTCCTTCAATAGGAC CTTCCTGATG ATGATTACAA
ATAAAGCCAC AGACGGTATT CTCTTTCTAGGGAAAGTGGA AAATCCCACT
AAATCCTAG
SEQ ID No. 28
SerpinA10 transcript variant 1 (Human) Protein
MKVVPSLLLSVLLAQVWLVPGLAPSPQSPETPAPQNQTSRVVQAPKEEEEDEQE
ASEEKASEEEKAWLMASRQQLAKETSNFGFSLLRKISMRHDGNMVFSPFGMSLA
MTGLMLGATGPTETQIKRGLHLQALKPTKPGLLPSLFKGLRETLSRNLELGLTQG
SFAFIHKDFDVKETFFNLSKRYFDTECVPMNFRNASQAKRLMNHYINKETRGKIP
KLFDEINPETKLILVDYILFKGKWLTPFDPVFTEVDTFHLDKYKTIKVPMMYGAG
KFASTFDKNFRCHVLKLPYQGNATMLVVLMEKMGDHLALEDYLTTDLVETWLR
NMKTRNMEVFFPKFKLDQKYEMHELLRQMGIRRIFSPFADLSELSATGRNLQVS
RVLQRTVIEVDERGTEAVAGILSEITAYSMPPVIKVDRPFHFMIYEETSGMLLFLG
RVVNPTLL
SEQ ID No. 29
SerpinA10 transcript variant 1 (Human) cDNA
ATGA AGGTGGTGCCAAGTCTCCTG CTCTCCGTCC TCCTGGCACA
GGTGTGGCTG GTACCCGGCT TGGCCCCCAGTCCTCAGTCG CCAGAGACCC
CAGCCCCTCA GAACCAGACC AGCAGGGTAG TGCAGGCTCCCAAGGAGGAA
GAGGAAGATG AGCAGGAGGC CAGCGAGGAG AAGGCCAGTAGGAAGAGAA
AGCCTGGCTG ATGGCCAGCA GGCAGCAGCT TGCCAAGGAG ACTTCAAACT
AAGATCTCCA TGAGGCACGA TGGCAACATG
GTCTTCTCTC CATTTGGCATGTCCTTGGCC ATGACAGGCT TGATGCTGGG
GGCCACAGGG CCGACTGAAA CCCAGATCAAGAGAGGGCTC CACTTGCAGG
CCCTGAAGCC CACCAAGCCC GGGCTCCTGC CTTCCCTCTTTAAGGGACTC
AGAGAGACCC TCTCCCGCAA CCTGGAACTG GGCCTCACAC AGGGGAGTTT
TGCCTTCATC CACAAGGATT TTGATGTCAA AGAGACTTTC TTCAATTTAT
CCAAGAGGTATTTTGATACA GAGTGCGTGC CTATGAATTT TCGCAATGCC
TCACAGGCCA AAAGGCTCATGAATCATTAC ATTAACAAAG AGACTCGGGG
GAAAATTCCC AAACTGTTTG ATGAGATTAATCCTGAAACC AAATTAATTC
TTGTGGATTA CATCTTGTTC AAAGGGAAAT GGTTGACCCCATTTGACCCT
GTCTTCACCG AAGTCGACAC TTTCCACCTG GACAAGTACA AGACCATTAA
GGTGCCCATG ATGTACGGTG CAGGCAAGTT TGCCTCCACC TTTGACAAGA
ATTTTCGTTGTCATGTCCTC AAACTGCCCT ACCAAGGAAA TGCCACCATG
CTGGTGGTCC TCATGGAGAAAATGGGTGAC CACCTCGCCC TTGAAGACTA
CCTGACCACA GACTTGGTGG AGACATGGCTCAGAAACATG AAAACCAGAA
ACATGGAAGT TTTCTTTCCG AAGTTCAAGC TAGATCAGAAGTATGAGATG
CATGAGCTGC TTAGGCAGAT GGGAATCAGA AGAATCTTCT CACCCTTTGC
TGACCTTAGT GAACTCTCAG CTACTGGAAG AAATCTCCAA GTATCCAGGG
TTTTACAAAGAACAGTGATT GAAGTTGATG AAAGGGGCAC TGAGGCAGTG
GCAGGAATCT TGTCAGAAATTACTGCTTAT TCCATGCCTC CTGTCATCAA
AGTGGACCGG CCATTTCATT TCATGATCTATGAAGAAACC TCTGGAATGC
TTCTGTTTCT GGGCAGGGTG GTGAATCCGA CTCTCCTATAA
SEQ ID No. 30
SerpinA10 transcript variant 2 (Human) Protein
MKVVPSLLLSVLLAQVWLVPGLAPSPQSPETPAPQNQTSRVVQAPKEEEEDEQE
ASEEKASEEEKAWLMASRQQLAKETSNFGFSLLRKISMRHDGNMVFSPFGMSLA
MTGLMLGATGPTETQIKRGLHLQALKPTKPGLLPSLFKGLRETLSRNLELGLTQG
SFAFIHKDFDVKETFFNLSKRYFDTECVPMNFRNASQAKRLMNHYINKETRGKIP
KLFDEINPETKLILVDYILFKGKWLTPFDPVFTEVDTFHLDKYKTIKVPMMYGAG
KFASTFDKNFRCHVLKLPYQGNATMLVVLMEKMGDHLALEDYLTTDLVETWLR
NMKTRNMEVFFPKFKLDQKYEMHELLRQMGIRRIFSPFADLSELSATGRNLQVS
RVLQRTVIEVDERGTEAVAGILSEITAYSMPPVIKVDRPFHFMIYEETSGMLLFLG
RVVNPTLL
SEQ ID No. 31
SerpinA10 transcript variant 2 (Human) cDNA
ATGAAGG TGGTGCCAAG TCTCCTGCTC TCCGTCCTCC TGGCACAGGT
GTGGCTGGTA CCCGGCTTGG CCCCCAGTCC TCAGTCGCCA GAGACCCCAG
CCCCTCAGAACCAGACCAGC AGGGTAGTGC AGGCTCCCAA GGAGGAAGAG
GAAGATGAGC AGGAGGCCAGCGAGGAGAAG GCCAGTGAGGAAGAGAAAGC
CTGGCTGATG GCCAGCAGGC AGCAGCTTGCCAAGGAGACT TCAAACTTCG
GATTCAGCCT GCTGCGAAAG ATCTCCATGA GGCACGATGGCAACATGGTC
TTCTCTCCAT TTGGCATGTC CTTGGCCATG ACAGGCTTGA TGCTGGGGGC
CACAGGGCCG ACTGAAACCC AGATCAAGAG AGGGCTCCAC TTGCAGGCCC
TGAAGCCCACCAAGCCCGGG CTCCTGCCTT CCCTCTTTAA GGGACTCAGA
GAGACCCTCT CCCGCAACCTGGAACTGGGC CTCACACAGG GGAGTTTTGC
CTTCATCCAC AAGGATTTTG ATGTCAAAGAGACTTTCTTC AATTTATCCA
AGAGGTATTT TGATACAGAG TGCGTGCCTA TGAATTTTCGCAATGCCTCA
CAGGCCAAAA GGCTCATGAA TCATTACATT AACAAAGAGA CTCGGGGGAA
AATTCCCAAA CTGTTTGATG AGATTAATCC TGAAACCAAA TTAATTCTTG
TGGATTACATCTTGTTCAAA GGGAAATGGT TGACCCCATT TGACCCTGTC
TTCACCGAAG TCGACACTTTCCACCTGGAC AAGTACAAGA CCATTAAGGT
GCCCATGATG TACGGTGCAG GCAAGTTTGCCTCCACCTTT GACAAGAATT
TTCGTTGTCA TGTCCTCAAA CTGCCCTACC AAGGAAATGCCACCATGCTG
GTGGTCCTCA TGGAGAAAAT GGGTGACCAC CTCGCCCTTG AAGACTACCT
GACCACAGAC TTGGTGGAGA CATGGCTCAG AAACATGAAA ACCAGAAACA
TGGAAGTTTTCTTTCCGAAG TTCAAGCTAG ATCAGAAGTA TGAGATGCAT
GAGCTGCTTA GGCAGATGGGAATCAGAAGA ATCTTCTCAC CCTTTGCTGA
CCTTAGTGAA CTCTCAGCTA CTGGAAGAAATCTCCAAGTA TCCAGGGTTT
TACAAAGAAC AGTGATTGAA GTTGATGAAA GGGGCACTGAGGCAGTGGCA
GGAATCTTGT CAGAAATTAC TGCTTATTCC ATGCCTCCTG TCATCAAAGT
GGACCGGCCA TTTCATTTCA TGATCTATGA AGAAACCTCT GGAATGCTTC
TGTTTCTGGGCAGGGTGGTG AATCCGACTC TCCTATAA
SEQ ID No. 32
SerpinA12 transcript variant 1 (Human) Protein
MNPTLGLAIFLAVLLTVKGLLKPSFSPRNYKALSEVQGWKQRMAAKELARQNM
DLGFKLLKKLAFYNPGRNIFLSPLSISTAFSMLCLGAQDSTLDEIKQGFNFRKMPE
KDLHEGFHYIIHELTQKTQDLKLSIGNTLFIDQRLQPQRKFLEDAKNFYSAETILTN
FQNLEMAQKQINDFISQKTHGKINNLIENIDPGTVMLLANYIFFRARWKHEFDPN
VTKEEDFFLEKNSSVKVPMMFRSGIYQVGYDDKLSCTILEIPYQKNITAIFILPD
EGKLKHLEKGLQVDTFSRWKTLLSRRVVDVSVPRLHMTGTFDLKKTLSYIGVSKI
FEEHGDLTKIAPHRSLKVGEAVHKAELKMDERGTEGAAGTGAQTLPMETPLVVK
IDKPYLLLIYSEKIPSVLFLGKIVNPIGK
SEQ ID No. 33
SerpinA12 transcript variant 1 (Human) cDNA
ATGA ACCCCACACT AGGCCTGGCC ATTTTTCTGG CTGTTCTCCT
CACGGTGAAA GGTCTTCTAA AGCCGAGCTT CTCACCAAGG AATTATAAAG
CTTTGAGCGAGGTCCAAGGA TGGAAGCAAA GGATGGCAGC CAAGGAGCTT
GCAAGGCAGA ACATGGACTTAGGCTTTAAG CTGCTCAAGA AGCTGGCCTT
TTACAACCCT GGCAGGAACA TCTTCCTATCCCCCTTGAGC ATCTCTACAG
CTTTCTCCAT GCTGTGCCTG GGTGCCCAGG ACAGCACCCTGGACGAGATC
AAGCAGGGGT TCAACTTCAG AAAGATGCCA GAAAAAGATC TTCATGAGGG
CTTCCATTAC ATCATCCACG AGCTGACCCA GAAGACCCAG GACCTCAAAC
TGAGCATTGGGAACACGCTG TTCATTGACC AGAGGCTGCA GCCACAGCGT
AAGTTTTTGG AAGATGCCAAGAACTTTTAC AGTGCCGAAA CCATCCTTAC
CAACTTTCAG AATTTGGAAA TGGCTCAGAAGCAGATCAAT GACTTTATCA
GTCAAAAAAC CCATGGGAAA ATTAACAACC TGATCGAGAATATAGACCCC
GGCACTGTGA TGCTTCTTGC AAATTATATT TTCTTTCGAG CCAGGTGGAA
ACATGAGTTT GATCCAAATG TAACTAAAGA GGAAGATTTC TTTCTGGAGA
AAAACAGTTCAGTCAAGGTG CCCATGATGT TCCGTAGTGG CATATACCAA
GTTGGCTATG ACGATAAGCTCTCTTGCACC ATCCTGGAAA TACCCTACCA
GAAAAATATC ACAGCCATCT TCATCCTTCCTGATGAGGGC AAGCTGAAGC
ACTTGGAGAA GGGATTGCAG GTGGACACTT TCTCCAGATGGAAAACATTA
CTGTCACGCA GGGTCGTAGA CGTGTCTGTA CCCAGACTCC ACATGACGGG
CACCTTCGAC CTGAAGAAGA CTCTCTCCTA CATAGGTGTC TCCAAAATCT
TTGAGGAACATGGTGATCTC ACCAAGATCG CCCCTCATCG CAGCCTGAAA
GTGGGCGAGG CTGTGCACAAGGCTGAGCTG AAGATGGATG AGAGGGGTAC
GGAAGGGGCC GCTGGCACCG GAGCACAGACTCTGCCCATG GAGACACCAC
TCGTCGTCAA GATAGACAAA CCCTATCTGC TGCTGATTTACAGCGAGAAA
ATACCTTCCG TGCTCTTCCT GGGAAAGATT GTTAACCCTA TTGGAAAATA
A
SEQ ID No. 34
SerpinA12 transcript variant 2 (Human) Protein
MNPTLGLAIFLAVLLTVKGLLKPSFSPRNYKALSEVQGWKQRMAAKELARQNM
DLGFKLLKKLAFYNPGRNIFLSPLSISTAFSMLCLGAQDSTLDEIKQGFNFRKMPE
KDLHEGFHYIIHELTQKTQDLKLSIGNTLFIDQRLQPQRKFLEDAKNFYSAETILTN
FQNLEMAQKQINDFISQKTHGKINNLIENIDPGTVMLLANYIFFRARWKHEFDPN
VTKEEDFFLEKNSSVKVPMMFRSGIYQVGYDDKLSCTILEIPYQKNITAIFILPDEG
KLKHLEKGLQVDTFSRWKTLLSRRVVDVSVPRLHMTGTFDLKKTLSYIGVSKIFE
EHGDLTKIAPHRSLKVGEAVHKAELKMDERGTEGAAGTGAQTLPMETPLVVKID
KPYLLLIYSEKIPSVLFLGKIVNPIGK
SEQ ID No. 35
SerpinA12 transcript variant 2 (Human) cDNA
ATGAACC CCACACTAGGCCTGGCCATT TTTCTGGCTG TTCTCCTCAC
GGTGAAAGGT CTTCTAAAGC CGAGCTTCTCACCAAGGAAT TATAAAGCTT
TGAGCGAGGT CCAAGGATGG AAGCAAAGGA TGGCAGCCAAGGAGCTTGCA
AGGCAGAACA TGGACTTAGG CTTTAAGCTG CTCAAGAAGC TGGCCTTTTA
CAACCCTGGC AGGAACATCT TCCTATCCCC CTTGAGCATC TCTACAGCTT
TCTCCATGCTGTGCCTGGGT GCCCAGGACA GCACCCTGGA CGAGATCAAG
CAGGGGTTCA ACTTCAGAAAGATGCCAGAA AAAGATCTTC ATGAGGGCTT
CCATTACATC ATCCACGAGC TGACCCAGAAGACCCAGGAC CTCAAACTGA
GCATTGGGAA CACGCTGTTC ATTGACCAGA GGCTGCAGCCACAGCGTAAG
TTTTTGGAAG ATGCCAAGAA CTTTTACAGT GCCGAAACCA TCCTTACCAA
CTTTCAGAAT TTGGAAATGG CTCAGAAGCA GATCAATGAC TTTATCAGTC
AAAAAACCCATGGGAAAATT AACAACCTGA TCGAGAATAT AGACCCCGGC
ACTGTGATGC TTCTTGCAAATTATATTTTC TTTCGAGCCA GGTGGAAACA
TGAGTTTGAT CCAAATGTAA CTAAAGAGGAAGATTTCTTT CTGGAGAAAA
ACAGTTCAGT CAAGGTGCCC ATGATGTTCC GTAGTGGCATATACCAAGTT
GGCTATGACG ATAAGCTCTC TTGCACCATC CTGGAAATAC CCTACCAGAA
AAATATCACA GCCATCTTCA TCCTTCCTGA TGAGGGCAAG CTGAAGCACT
TGGAGAAGGGATTGCAGGTG GACACTTTCT CCAGATGGAA AACATTACTG
TCACGCAGGG TCGTAGACGTGTCTGTACCC AGACTCCACA TGACGGGCAC
CTTCGACCTG AAGAAGACTC TCTCCTACATAGGTGTCTCC AAAATCTTTG
AGGAACATGG TGATCTCACC AAGATCGCCC CTCATCGCAGCCTGAAAGTG
GGCGAGGCTG TGCACAAGGC TGAGCTGAAG ATGGATGAGA GGGGTACGGA
AGGGGCCGCT GGCACCGGAG CACAGACTCT GCCCATGGAG ACACCACTCG
TCGTCAAGATAGACAAACCC TATCTGCTGC TGATTTACAG CGAGAAAATA
CCTTCCGTGC TCTTCCTGGGAAAGATTGTT AACCCTATTG GAAAATAA
SEQ ID No. 36
SerpinB1 (Human) Protein
MEQLSSANTRFALDLFLALSENNPAGNIFISPFSISSAMAMVFLGTRGNTAAQLSK
TFHFNTVEEVHSRFQSLNADINKRGASYILKLANRLYGEKTYNFLPEFLVSTQKT
YGADLASVDFQHASEDARKTINQWVKGQTEGKIPELLASGMVDNMTKLVLVNA
IYFKGNWKDKFMKEATTNAPFRLNKKDRKTVKMMYQKKKFAYGYIEDLKCRV
LELPYQGEELSMVILLPDDIEDESTGLKKIEEQLTLEKLHEWTKPENLDFIEVNVSL
PRFKLEESYTLNSDLARLGVQDLFNSSKADLSGMSGARDIFISKIVHKSFVEVNEE
GTEAAAATAGIATFCMLMPEENFTADHPFLFFIRHNSSGSILFLGRFSSP
SEQ ID No. 37
SerpinB1 (Human) cDNA
ATGG AGCAGCTGAG CTCAGCAAAC ACCCGCTTCG CCTTGGACCT
GTTCCTGGCGTTGAGTGAGA ACAATCCGGC TGGAAACATC TTCATCTCTC
CCTTCAGCAT TTCATCTGCTATGGCCATGG TTTTTCTGGG GACCAGAGGT
AACACGGCAG CACAGCTGTC CAAGACTTTCCATTTCAACA CGGTTGAAGA
GGTTCATTCA AGATTCCAGA GTCTGAATGC TGATATCAACAAACGTGGAG
CGTCTTATAT TCTGAAACTT GCTAATAGAT TATATGGAGA GAAAACTTAC
AATTTCCTTC CTGAGTTCTT GGTTTCGACT CAGAAAACAT ATGGTGCTGA
CCTGGCCAGTGTGGATTTTC AGCATGCCTC TGAAGATGCA AGGAAGACCA
TAAACCAGTG GGTCAAAGGACAGACAGAAG GAAAAATTCC GGAACTGTTG
GCTTCGGGCA TGGTTGATAA CATGACCAAACTTGTGCTAG TAAATGCCAT
CTATTTCAAG GGAAACTGGA AGGATAAATT CATGAAAGAAGCCACGACGA
ATGCACCATT CAGATTGAAT AAGAAAGACA GAAAAACTGT GAAAATGATG
TATCAGAAGA AAAAATTTGC ATATGGCTAC ATCGAGGACC TTAAGTGCCG
TGTGCTGGAACTGCCTTACC AAGGCGAGGA GCTCAGCATG GTCATCCTGC
TGCCGGATGA CATTGAGGACGAGTCCACGG GCCTGAAGAA GATTGAGGAA
CAGTTGACTT TGGAAAAGTT GCATGAGTGGACTAAACCTG AGAATCTCGA
TTTCATTGAA GTTAATGTCA GCTTGCCCAG GTTCAAACTGGAAGAGAGTT
ACACTCTCAA CTCCGACCTC GCCCGCCTAG GTGTGCAGGA TCTCTTTAAC
AGTAGCAAGG CTGATCTGTC TGGCATGTCA GGAGCCAGAG ATATTTTTAT
ATCAAAAATTGTCCACAAGT CATTTGTGGA AGTGAATGAA GAGGGAACAG
AGGCGGCAGC TGCCACAGCAGGCATCGCAA CTTTCTGCAT GTTGATGCCC
GAAGAAAATT TCACTGCCGA CCATCCATTCCTTTTCTTTA TTCGGCATAA
TTCCTCAGGT AGCATCCTAT TCTTGGGGAG ATTTTCTTCCCCTTAG
SEQ ID No. 38
SerpinB6 variant 1 (Human) Protein
MDVLAEANGTFALNLLKTLGKDNSKNVFFSPMSMSCALAMVYMGAKGNTAAQ
MAQILSFNKSGGGGDIHQGFQSLLTEVNKTGTQYLLRMANRLFGEKSCDFLSSFR
DSCQKFYQAEMEELDFISAVEKSRKHINTWVAEKTEGKIAELLSPGSVDPLTRLV
LVNAVYFRGNWDEQFDKENTEERLFKVSKNEEKPVQMMFKQSTFKKTYIGEIFT
QILVLPYVGKELNMIIMLPDETTDLRTVEKELTYEKFVEWTRLDMMDEEEVEVSL
PRFKLEESYDMESVLRNLGMTDAFELGKADFSGMSQTDLSLSKVVHKSFVEVNE
EGTEAAAATAAIMMMRCARFVPRFCADHPFLFFIQHSKTNGILFCGRFSSP
SEQ ID No. 39
SerpinB6 variant 1 (Human) cDNA
ATGGATG TTCTCGCAGAAGCAAATGGC ACCTTTGCCT TAAACCTTTT
GAAAACGCTG GGTAAAGACA ACTCGAAGAATGTGTTTTTC TCACCCATGA
GCATGTCCTG TGCCCTGGCC ATGGTCTACA TGGGGGCAAAGGGAAACACC
GCTGCACAGA TGGCCCAGAT ACTTTCTTTC AATAAAAGTG GCGGTGGTGG
AGACATCCAC CAGGGCTTCC AGTCTCTTCT CACCGAAGTG AACAAGACTG
GCACGCAGTACTTGCTTAGG ATGGCCAACA GGCTCTTTGG GGAAAAGTCT
TGTGATTTCC TCTCATCTTTTAGAGATTCC TGCCAAAAAT TCTACCAAGC
AGAGATGGAG GAGCTTGACT TTATCAGCGCCGTAGAGAAG TCCAGAAAAC
ACATAAACAC CTGGGTAGCT GAAAAGACAG AAGGTAAAATTGCGGAGTTG
CTCTCTCCGG GCTCAGTGGA TCCATTGACA AGGCTGGTTC TGGTGAATGC
TGTCTATTTC AGAGGAAACT GGGATGAACA GTTTGACAAG GAGAACACCG
AGGAGAGACTGTTTAAAGTC AGCAAGAATG AGGAGAAACC TGTGCAAATG
ATGTTTAAGC AATCTACTTTTAAGAAGACC TATATAGGAG AAATATTTAC
CCAAATCTTG GTGCTTCCAT ATGTTGGCAAGGAACTGAAT ATGATCATCA
TGCTTCCGGA CGAGACCACT GACTTGAGAA CGGTGGAGAAAGAACTCACT
TACGAGAAGT TCGTAGAATG GACGAGGCTG GACATGATGG ATGAAGAGGA
GGTGGAAGTG TCCCTCCCGC GGTTTAAACT AGAGGAAAGC TACGACATGG
AGAGTGTCCTGCGCAACCTG GGCATGACTG ATGCCTTCGA GCTGGGCAAG
GCAGACTTCT CTGGAATGTCCCAGACAGAC CTGTCTCTGT CCAAGGTCGT
GCACAAGTCT TTTGTGGAGG TCAATGAGGAAGGCACGGAG GCTGCAGCCG
CCACAGCTGC CATCATGATG ATGCGGTGTG CCAGATTCGTCCCCCGCTTC
TGCGCCGACC ACCCCTTCCT TTTCTTCATC CAGCACAGCA AGACCAACGG
GATTCTCTTC TGCGGCCGCT TTTCCTCTCC GTGA
SEQ ID No. 40
SerpinB6 variant 2 (Human) Protein
MSAIMDVLAEANGTFALNLLKTLGKDNSKNVFFSPMSMSCALAMVYMGAKGN
TAAQMAQILSFNKSGGGGDIHQGFQSLLTEVNKTGTQYLLRMANRLFGEKSCDF
LSSFRDSCQKFYQAEMEELDFISAVEKSRKHINTWVAEKTEGKIAELLSPGSVD
PLTRLVLVNAVYFRGNWDEQFDKENTEERLFKVSKNEEKPVQMMFKQSTFKKT
YIGEIFTQILVLPYVGKELNMIIMLPDETTDLRTVEKELTYEKFVEWTRLDMMDE
EEVEVSLPRFKLEESYDMESVLRNLGMTDAFELGKADFSGMSQTDLSLSKVVHK
SFVEVNEEGTEAAAATAAIMMMRCARFVPRFCADHPFLFFIQHSKTNGILFCGRF
SSP
SEQ ID No. 41
SerpinB6 variant 2 (Human) cDNA
ATGT CTGCCATCATGGATGTTCTC GCAGAAGCAA ATGGCAC CTT
TGCCTTAAAC CTTTTGAAAA CGCTGGGTAAAGACAACTCG AAGAATGTGT
TTTTCTCACC CATGAGCATG TCCTGTGCCC TGGCCATGGTCTACATGGGG
GCAAAGGGAA ACACCGCTGC ACAGATGGCC CAGATACTTT CTTTCAATAA
AAGTGGCGGT GGTGGAGACA TCCACCAGGG CTTCCAGTCT CTTCTCACCG
AAGTGAACAAGACTGGCACG CAGTACTTGC TTAGGATGGC CAACAGGCTC
TTTGGGGAAA AGTCTTGTGATTTCCTCTCA TCTTTTAGAG ATTCCTGCCA
AAAATTCTAC CAAGCAGAGA TGGAGGAGCTTGACTTTATC AGCGCCGTAG
AGAAGTCCAG AAAACACATA AACACCTGGG TAGCTGAAAAGACAGAAGGT
AAAATTGCGG AGTTGCTCTC TCCGGGCTCA GTGGATCCAT TGACAAGGCT
GGTTCTGGTG AATGCTGTCT ATTTCAGAGG AAACTGGGAT GAACAGTTTG
ACAAGGAGAACACCGAGGAG AGACTGTTTA AAGTCAGCAA GAATGAGGAG
AAACCTGTGC AAATGATGTTTAAGCAATCT ACTTTTAAGA AGACCTATAT
AGGAGAAATA TTTACCCAAA TCTTGGTGCTTCCATATGTT GGCAAGGAAC
TGAATATGAT CATCATGCTT CCGGACGAGA CCACTGACTTGAGAACGGTG
GAGAAAGAAC TCACTTACGA GAAGTTCGTA GAATGGACGA GGCTGGACAT
GATGGATGAA GAGGAGGTGG AAGTGTCCCT CCCGCGGTTT AAACTAGAGG
AAAGCTACGACATGGAGAGT GTCCTGCGCA ACCTGGGCAT GACTGATGCC
TTCGAGCTGG GCAAGGCAGACTTCTCTGGA ATGTCCCAGA CAGACCTGTC
TCTGTCCAAG GTCGTGCACA AGTCTTTTGTGGAGGTCAAT GAGGAAGGCA
CGGAGGCTGC AGCCGCCACA GCTGCCATCA TGATGATGCGGTGTGCCAGA
TTCGTCCCCC GCTTCTGCGC CGACCACCCC TTCCTTTTCT TCATCCAGCA
CAGCAAGACC AACGGGATTC TCTTCTGCGG CCGCTTTTCC TCTCCGTGA
SEQ ID No. 42
SerpinB6 variant 3 (Human) Protein
MGAAQSLPGHRSAIMDVLAEANGTFALNLLKTLGKDNSKNVFFSPMSMSCALA
MVYMGAKGNTAAQMAQILSFNKSGGGGDIHQGFQSLLTEVNKTGTQYLL
RMANRLFGEKSCDFLSSFRDSCQKFYQAEMEELDFISAVEKSRKHINTWVAEKTE
GKIAELLSPGSVDPLTRLVLVNAVYFRGNWDEQFDKENTEERLFKVSKNEEKPV
QMMFKQSTFKKTYIGEIFTQILVLPYVGKELNMIIMLPDETTDLRTVEKELTYEKF
VEWTRLDMMDEEEVEVSLPRFKLEESYDMESVLRNLGMTDAFELGKADFSGMS
QTDLSLSKVVHKSFVEVNEEGTEAAAATAAIMMMRCARFVPRFCADHPFLFFIQ
HSKTNGILFCGRFSSP
SEQ ID No. 43
SerpinB6 variant 3 (Human) cDNA
ATGGGGGCG GCGCAGAGCCTCCCGGGCCA CAGGTCTGCC ATCATGGATG
TTCTCGCAGA AGCAAATGGC ACCTTTGCCTTAAACCTTTT GAAAACGCTG
GGTAAAGACA ACTCGAAGAA TGTGTTTTTC TCACCCATGAGCATGTCCTG
TGCCCTGGCC ATGGTCTACA TGGGGGCAAA GGGAAACACC GCTGCACAGA
TGGCCCAGAT ACTTTCTTTC AATAAAAGTG GCGGTGGTGG AGACATCCAC
CAGGGCTTCCAGTCTCTTCT CACCGAAGTG AACAAGACTG GCACGCAGTA
CTTGCTTAGG ATGGCCAACAGGCTCTTTGG GGAAAAGTCT TGTGATTTCC
TCTCATCTTT TAGAGATTCC TGCCAAAAATTCTACCAAGC AGAGATGGAG
GAGCTTGACT TTATCAGCGC CGTAGAGAAG TCCAGAAAACACATAAACAC
CTGGGTAGCT GAAAAGACAG AAGGTAAAAT TGCGGAGTTG CTCTCTCCGG
GCTCAGTGGA TCCATTGACA AGGCTGGTTC TGGTGAATGC TGTCTATTTC
AGAGGAAACTGGGATGAACA GTTTGACAAG GAGAACACCG AGGAGAGACT
GTTTAAAGTC AGCAAGAATGAGGAGAAACC TGTGCAAATG ATGTTTAAGC
AATCTACTTT TAAGAAGACC TATATAGGAGAAATATTTAC CCAAATCTTG
GTGCTTCCAT ATGTTGGCAA GGAACTGAAT ATGATCATCATGCTTCCGGA
CGAGACCACT GACTTGAGAA CGGTGGAGAA AGAACTCACT TACGAGAAGT
TCGTAGAATG GACGAGGCTG GACATGATGG ATGAAGAGGA GGTGGAAGTG
TCCCTCCCGCGGTTTAAACT AGAGGAAAGC TACGACATGG AGAGTGTCCT
GCGCAACCTG GGCATGACTGATGCCTTCGA GCTGGGCAAG GCAGACTTCT
CTGGAATGTC CCAGACAGAC CTGTCTCTGTCCAAGGTCGT GCACAAGTCT
TTTGTGGAGG TCAATGAGGA AGGCACGGAG GCTGCAGCCGCCACAGCTGC
CATCATGATG ATGCGGTGTG CCAGATTCGT CCCCCGCTTC TGCGCCGACC
ACCCCTTCCT TTTCTTCATC CAGCACAGCA AGACCAACGG GATTCTCTTC
TGCGGCCGCTTTTCCTCTCC GTGA
SEQ ID No. 44
SerpinB6 variant 4 (Human) Protein
MSSRQRGNFNYKLAFKSAIMDVLAEANGTFALNLLKTLGKDNSKNVFFSPMSMS
CALAMVYMGAKGNTAAQMAQILSFNKSGGGGDIHQGFQSLLTEVNKTG
TQYLLRMANRLFGEKSCDFLSSFRDSCQKFYQAEMEELDFISAVEKSRKHINTWV
AEKTEGKIAELLSPGSVDPLTRLVLVNAVYFRGNWDEQFDKENTEERLFKVSKN
EEKPVQMMFKQSTFKKTYIGEIFTQILVLPYVGKELNMIIMLPDETTDLRTVEKEL
TYEKFVEWTRLDMMDEEEVEVSLPRFKLEESYDMESVLRNLGMTDAFELGKAD
FSGMSQTDLSLSKVVHKSFVEVNEEGTEAAAATAAIMMMRCARFVPRFCADHPF
LFFIQHSKTNGILFCGRFSSP
SEQ ID No. 45
SerpinB6 variant 4 (Human) cDNA
ATGTCT TCAAGGCAAA GAGGAAACTT TAACTACAAA TTGGCATTTA
AGTCTGCCATCATGGATGTT CTCGCAGAAG CAAATGGCAC CTTTGCCTTA
AACCTTTTGA AAACGCTGGGTAAAGACAAC TCGAAGAATG TGTTTTTCTC
ACCCATGAGC ATGTCCTGTG CCCTGGCCATGGTCTACATG GGGGCAAAGG
GAAACACCGC TGCACAGATG GCCCAGATAC TTTCTTTCAATAAAAGTGGC
GGTGGTGGAG ACATCCACCA GGGCTTCCAG TCTCTTCTCA CCGAAGTGAA
CAAGACTGGC ACGCAGTACT TGCTTAGGAT GGCCAACAGG CTCTTTGGGG
AAAAGTCTTGTGATTTCCTC TCATCTTTTA GAGATTCCTG CCAAAAATTC
TACCAAGCAG AGATGGAGGAGCTTGACTTT ATCAGCGCCG TAGAGAAGTC
CAGAAAACAC ATAAACACCT GGGTAGCTGAAAAGACAGAA GGTAAAATTG
CGGAGTTGCT CTCTCCGGGC TCAGTGGATC CATTGACAAGGCTGGTTCTG
GTGAATGCTG TCTATTTCAG AGGAAACTGG GATGAACAGT TTGACAAGGA
GAACACCGAG GAGAGACTGT TTAAAGTCAG CAAGAATGAG GAGAAACCTG
TGCAAATGATGTTTAAGCAA TCTACTTTTA AGAAGACCTA TATAGGAGAA
ATATTTACCC AAATCTTGGTGCTTCCATAT GTTGGCAAGG AACTGAATAT
GATCATCATG CTTCCGGACG AGACCACTGACTTGAGAACG GTGGAGAAAG
AACTCACTTA CGAGAAGTTC GTAGAATGGA CGAGGCTGGACATGATGGAT
GAAGAGGAGG TGGAAGTGTC CCTCCCGCGG TTTAAACTAG AGGAAAGCTA
CGACATGGAG AGTGTCCTGC GCAACCTGGG CATGACTGAT GCCTTCGAGC
TGGGCAAGGCAGACTTCTCT GGAATGTCCC AGACAGACCT GTCTCTGTCC
AAGGTCGTGC ACAAGTCTTTTGTGGAGGTC AATGAGGAAG GCACGGAGGC
TGCAGCCGCC ACAGCTGCCA TCATGATGATGCGGTGTGCC AGATTCGTCC
CCCGCTTCTG CGCCGACCAC CCCTTCCTTT TCTTCATCCAGCACAGCAAG
ACCAACGGGA TTCTCTTCTG CGGCCGCTTT TCCTCTCCGT GA
SEQ ID No. 46
SerpinB6 variant 5 (Human) Protein
MDVLAEANGTFALNLLKTLGKDNSKNVFFSPMSMSCALAMVYMGAKGNTAAQ
MAQILSFNKSGGGGDIHQGFQSLLTEVNKTGTQYLLRMANRLFGEKSCDFLSSFR
DSCQKFYQAEMEELDFISAVEKSRKHINTWVAEKTEGKIAELLSPGSVDPLTR
LVLVNAVYFRGNWDEQFDKENTEERLFKVSKNEEKPVQMMFKQSTFKKTYIGEI
FTQILVLPYVGKELNMIIMLPDETTDLRTVEKELTYEKFVEWTRLDMMDEEEVEV
SLPRFKLEESYDMESVLRNLGMTDAFELGKADFSGMSQTDLSLSKVVHKSFVEV
NEEGTEAAAATAAIMMMRCARFVPRFCADHPFLFFIQHSKTNGILFCGRFSSP
SEQ ID No. 47
SerpinB6 variant 5 (Human) cDNA
ATGGA TGTTCTCGCA GAAGCAAATG GCACCTTTGCCTTAAACCTT
TTGAAAACGC TGGGTAAAGA CAACTCGAAG AATGTGTTTT TCTCACCCAT
GAGCATGTCC TGTGCCCTGG CCATGGTCTA CATGGGGGCA AAGGGAAACA
CCGCTGCACAGATGGCCCAG ATACTTTCTT TCAATAAAAG TGGCGGTGGT
GGAGACATCC ACCAGGGCTTCCAGTCTCTT CTCACCGAAG TGAACAAGAC
TGGCACGCAG TACTTGCTTA GGATGGCCAACAGGCTCTTT GGGGAAAAGT
CTTGTGATTT CCTCTCATCT TTTAGAGATT CCTGCCAAAAATTCTACCAA
GCAGAGATGG AGGAGCTTGA CTTTATCAGC GCCGTAGAGA AGTCCAGAAA
ACACATAAAC ACCTGGGTAG CTGAAAAGAC AGAAGGTAAA ATTGCGGAGT
TGCTCTCTCCGGGCTCAGTG GATCCATTGA CAAGGCTGGT TCTGGTGAAT
GCTGTCTATT TCAGAGGAAACTGGGATGAA CAGTTTGACA AGGAGAACAC
CGAGGAGAGA CTGTTTAAAG TCAGCAAGAA
TGAGGAGAAA CCTGTGCAAA TGATGTTTAA GCAATCTACT TTTAAGAAGA
CCTATATAGGAGAAATATTT ACCCAAATCT TGGTGCTTCC ATATGTTGGC
AAGGAACTGA ATATGATCATCATGCTTCCG GACGAGACCA CTGACTTGAG
AACGGTGGAG AAAGAACTCA CTTACGAGAAGTTCGTAGAA TGGACGAGGC
TGGACATGAT GGATGAAGAG GAGGTGGAAG TGTCCCTCCCGCGGTTTAAA
CTAGAGGAAA GCTACGACAT GGAGAGTGTC CTGCGCAACC TGGGCATGAC
TGATGCCTTC GAGCTGGGCA AGGCAGACTT CTCTGGAATG TCCCAGACAG
ACCTGTCTCTGTCCAAGGTC GTGCACAAGT CTTTTGTGGA GGTCAATGAG
GAAGGCACGG AGGCTGCAGCCGCCACAGCT GCCATCATGA TGATGCGGTG
TGCCAGATTC GTCCCCCGCT TCTGCGCCGACCACCCCTTC CTTTTCTTCA
TCCAGCACAG CAAGACCAAC GGGATTCTCT TCTGCGGCCGCTTTTCCTCT
CCGTGA
SEQ ID No. 48
SerpinB6 variant 6 (Human) Protein
MDVLAEANGTFALNLLKTLGKDNSKNVFFSPMSMSCALAMVYMGAKGNTAAQ
MAQILSFNKSGGGGDIHQGFQSLLTEVNKTGTQYLLRMANRLFGEKSCDF
LSSFRDSCQKFYQAEMEELDFISAVEKSRKHINTWVAEKTEGKIAELLSPGSVDPL
TRLVLVNAVYFRGNWDEQFDKENTEERLFKVSKNEEKPVQMMFKQSTFKKTYI
GEIFTQILVLPYVGKELNMIIMLPDETTDLRTVEKELTYEKFVEWTRLDMMDEEE
VEVSLPRFKLEESYDMESVLRNLGMTDAFELGKADFSGMSQTDLSLSKVVHKSF
VEVNEEGTEAAAATAAIMMMRCARFVPRFCADHPFLFFIQHSKTNGILFCGRFSS
P
SEQ ID No. 49
SerpinB6 variant 6 (Human) cDNA
ATG GATGTTCTCGCAGAAGCAAA TGGCACCTTT GCCTTAAACC
TTTTGAAAAC GCTGGGTAAA GACAACTCGAAGAATGTGTT TTTCTCACCC
ATGAGCATGT CCTGTGCCCT GGCCATGGTC TACATGGGGGCAAAGGGAAA
CACCGCTGCA CAGATGGCCC AGATACTTTC TTTCAATAAA AGTGGCGGTG
GTGGAGACAT CCACCAGGGC TTCCAGTCTC TTCTCACCGA AGTGAACAAG
ACTGGCACGCAGTACTTGCT TAGGATGGCC AACAGGCTCT TTGGGGAAAA
GTCTTGTGAT TTCCTCTCATCTTTTAGAGA TTCCTGCCAA AAATTCTACC
AAGCAGAGAT GGAGGAGCTT GACTTTATCAGCGCCGTAGA GAAGTCCAGA
AAACACATAA ACACCTGGGT AGCTGAAAAG ACAGAAGGTAAAATTGCGGA
GTTGCTCTCT CCGGGCTCAG TGGATCCATT GACAAGGCTG GTTCTGGTGA
ATGCTGTCTA TTTCAGAGGA AACTGGGATG AACAGTTTGA CAAGGAGAAC
ACCGAGGAGAGACTGTTTAA AGTCAGCAAG AATGAGGAGA AACCTGTGCA
AATGATGTTT AAGCAATCTACTTTTAAGAA GACCTATATA GGAGAAATAT
TTACCCAAAT CTTGGTGCTT CCATATGTTGGCAAGGAACT GAATATGATC
ATCATGCTTC CGGACGAGAC CACTGACTTG AGAACGGTGGAGAAAGAACT
CACTTACGAG AAGTTCGTAG AATGGACGAG GCTGGACATG ATGGATGAAG
AGGAGGTGGA AGTGTCCCTC CCGCGGTTTA AACTAGAGGA AAGCTACGAC
ATGGAGAGTGTCCTGCGCAA CCTGGGCATG ACTGATGCCT TCGAGCTGGG
CAAGGCAGAC TTCTCTGGAATGTCCCAGAC AGACCTGTCT CTGTCCAAGG
TCGTGCACAA GTCTTTTGTG GAGGTCAATGAGGAAGGCAC GGAGGCTGCA
GCCGCCACAG CTGCCATCAT GATGATGCGG TGTGCCAGATTCGTCCCCCG
CTTCTGCGCC GACCACCCCT TCCTTTTCTT CATCCAGCAC AGCAAGACCA
ACGGGATTCT CTTCTGCGGC CGCTTTTCCT CTCCGTGA
SEQ ID No. 50
SerpinB6 variant 7 (Human) Protein
MDVLAEANGTFALNLLKTLGKDNSKNVFFSPMSMSCALAMVYMGAKGNTAAQ
MAQILSFNKSGGGGDIHQGFQSLLTEVNKTGTQYLLRMANRLFGEKSCDF
LSSFRDSCQKFYQAEMEELDFISAVEKSRKHINTWVAEKTEGKIAELLSPGSVDPL
TRLVLVNAVYFRGNWDEQFDKENTEERLFKVSKNEEKPVQMMFKQSTFKKTYI
GEIFTQILVLPYVGKELNMIIMLPDETTDLRTVEKELTYEKFVEWTRLDMMDEEE
VEVSLPRFKLEESYDMESVLRNLGMTDAFELGKADFSGMSQTDLSLSKVVHKSF
VEVNEEGTEAAAATAAIMMMRCARFVPRFCADHPFLFFIQHSKTNGILFCGRFSS
P
SEQ ID No. 51
SerpinB6 variant 7 (Human) cDNA
AT GGATGTTCTC GCAGAAGCAA ATGGCACCTTTGCCTTAAAC
CTTTTGAAAA CGCTGGGTAA AGACAACTCG AAGAATGTGT
TTTTCTCACCCATGAGCATG TCCTGTGCCC TGGCCATGGT CTACATGGGG
GCAAAGGGAA ACACCGCTGCACAGATGGCC CAGATACTTT CTTTCAATAA
AAGTGGCGGT GGTGGAGACA TCCACCAGGGCTTCCAGTCT CTTCTCACCG
AAGTGAACAA GACTGGCACG CAGTACTTGC TTAGGATGGCCAACAGGCTC
TTTGGGGAAA AGTCTTGTGA TTTCCTCTCA TCTTTTAGAG ATTCCTGCCA
AAAATTCTAC CAAGCAGAGA TGGAGGAGCT TGACTTTATC AGCGCCGTAG
AGAAGTCCAGAAAACACATA AACACCTGGG TAGCTGAAAA GACAGAAGGT
AAAATTGCGG AGTTGCTCTCTCCGGGCTCA GTGGATCCAT TGACAAGGCT
GGTTCTGGTG AATGCTGTCT ATTTCAGAGGAAACTGGGAT GAACAGTTTG
ACAAGGAGAA CACCGAGGAG AGACTGTTTA AAGTCAGCAAGAATGAGGAG
AAACCTGTGC AAATGATGTT TAAGCAATCT ACTTTTAAGA AGACCTATAT
AGGAGAAATA TTTACCCAAA TCTTGGTGCT TCCATATGTT GGCAAGGAAC
TGAATATGATCATCATGCTT CCGGACGAGA CCACTGACTT GAGAACGGTG
GAGAAAGAAC TCACTTACGAGAAGTTCGTA GAATGGACGA GGCTGGACAT
GATGGATGAA GAGGAGGTGG AAGTGTCCCTCCCGCGGTTT AAACTAGAGG
AAAGCTACGA CATGGAGAGT GTCCTGCGCA ACCTGGGCATGACTGATGCC
TTCGAGCTGG GCAAGGCAGA CTTCTCTGGA ATGTCCCAGA CAGACCTGTC
TCTGTCCAAG GTCGTGCACA AGTCTTTTGT GGAGGTCAAT GAGGAAGGCA
CGGAGGCTGCAGCCGCCACA GCTGCCATCA TGATGATGCG GTGTGCCAGA
TTCGTCCCCC GCTTCTGCGCCGACCACCCC TTCCTTTTCT TCATCCAGCA
CAGCAAGACC AACGGGATTC TCTTCTGCGGCCGCTTTTCC TCTCCGTGA
SEQ ID No. 52
SerpinB6 variant 8 (Human) Protein
MDVLAEANGTFALNLLKTLGKDNSKNVFFSPMSMSCALAMVYMGAKGNTAAQ
MAQILSFNKSGGGGDIHQGFQSLLTEVNKTGTQYLLRMANRLFGEKSCDF
LSSFRDSCQKFYQAEMEELDFISAVEKSRKHINTWVAEKTEGKIAELLSPGSVDPL
TRLVLVNAVYFRGNWDEQFDKENTEERLFKVSKNEEKPVQMMFKQSTFKKTYI
GEIFTQILVLPYVGKELNMIIMLPDETTDLRTVEKELTYEKFVEWTRLDMMDEEE
VEVSLPRFKLEESYDMESVLRNLGMTDAFELGKADFSGMSQTDLSLSKVVHKSF
VEVNEEGTEAAAATAAIMMMRCARFVPRFCADHPFLFFIQHSKTNGILFCGRFSS
P
SEQ ID No. 53
SerpinB6 variant 8 (Human) cDNA
ATGGAT GTTCTCGCAGAAGCAAATGG CACCTTTGCC TTAAACCTTT
TGAAAACGCT GGGTAAAGAC AACTCGAAGAATGTGTTTTT CTCACCCATG
AGCATGTCCT GTGCCCTGGC CATGGTCTAC ATGGGGGCAAAGGGAAACAC
CGCTGCACAG ATGGCCCAGA TACTTTCTTT CAATAAAAGT GGCGGTGGTG
GAGACATCCA CCAGGGCTTC CAGTCTCTTC TCACCGAAGT GAACAAGACT
GGCACGCAGTACTTGCTTAG GATGGCCAAC AGGCTCTTTG GGGAAAAGTC
TTGTGATTTC CTCTCATCTTTTAGAGATTC CTGCCAAAAA TTCTACCAAG
CAGAGATGGA GGAGCTTGAC TTTATCAGCGCCGTAGAGAA GTCCAGAAAA
CACATAAACA CCTGGGTAGC TGAAAAGACA GAAGGTAAAA
TTGCGGAGTT GCTCTCTCCG GGCTCAGTGG ATCCATTGAC AAGGCTGGTT
CTGGTGAATGCTGTCTATTT CAGAGGAAAC TGGGATGAAC AGTTTGACAA
GGAGAACACC GAGGAGAGACTGTTTAAAGT CAGCAAGAAT GAGGAGAAAC
CTGTGCAAAT GATGTTTAAG CAATCTACTTTTAAGAAGAC CTATATAGGA
GAAATATTTA CCCAAATCTT GGTGCTTCCA TATGTTGGCAAGGAACTGAA
TATGATCATC ATGCTTCCGG ACGAGACCAC TGACTTGAGA ACGGTGGAGA
AAGAACTCAC TTACGAGAAG TTCGTAGAAT GGACGAGGCT GGACATGATG
GATGAAGAGGAGGTGGAAGT GTCCCTCCCG CGGTTTAAAC TAGAGGAAAG
CTACGACATG GAGAGTGTCCTGCGCAACCT GGGCATGACT GATGCCTTCG
AGCTGGGCAA GGCAGACTTC TCTGGAATGTCCCAGACAGA CCTGTCTCTG
TCCAAGGTCG TGCACAAGTC TTTTGTGGAG GTCAATGAGGAAGGCACGGA
GGCTGCAGCC GCCACAGCTG CCATCATGAT GATGCGGTGT
GCCAGATTCGTCCCCCGCTT CTGCGCCGAC CACCCCTTCC TTTTCTTCAT
CCAGCACAGC AAGACCAACGGGATTCTCTT CTGCGGCCGC TTTTCCTCTC
CGTGA
SEQ ID No. 54
SerpinB9 (Human) Protein
METLSNASGTFAIRLLKILCQDNPSHNVFCSPVSISSALAMVLLGAKGNTATQMA
QALSLNTEEDIHRAFQSLLTEVNKAGTQYLLRTANRLFGEKTCQFLSTFKESCLQF
YHAELKELSFIRAAEESRKHINTWVSKKTEGKIEELLPGSSIDAETRLVLVNAIYFK
GKWNEPFDETYTREMPFKINQEEQRPVQMMYQEATFKLAHVGEVRAQLLE
LPYARKELSLLVLLPDDGVELSTVEKSLTFEKLTAWTKPDCMKSTEVEVLLPKFK
LQEDVDMESVLRHLGIVDAFQQGKADLSAMSAERDLCLSKFVHKSFVEVNEEGT
EAAAASSCFVVAECCMESGPRFCADHPFLFFIRHNRANSILFCGRFSSP
SEQ ID No. 55
SerpinB9 (Human) cDNA
ATGGAAAC TCTTTCTAAT GCAAGTGGTA CTTTTGCCAT ACGCCTTTTA
AAGATACTGTGTCAAGATAA CCCTTCGCAC AACGTGTTCT GTTCTCCTGT
GAGCATCTCC TCTGCCCTGGCCATGGTTCT CCTAGGGGCA AAGGGAAACA
CCGCAACCCA GATGGCCCAG GCACTGTCTTTAAACACAGA GGAAGACATT
CATCGGGCTT TCCAGTCGCT TCTCACTGAA GTGAACAAGGCTGGCACACA
GTACCTGCTG AGAACGGCCA ACAGGCTCTT TGGAGAGAAA ACTTGTCAGT
TCCTCTCAAC GTTTAAGGAA TCCTGTCTTC AATTCTACCA TGCTGAGCTG
AAGGAGCTTTCCTTTATCAG AGCTGCAGAA GAGTCCAGGA AACACATCAA
CACCTGGGTC TCAAAAAAGACCGAAGGTAA AATTGAAGAG TTGTTGCCGG
GTAGCTCAAT TGATGCAGAA ACCAGGCTGGTTCTTGTCAA TGCCATCTAC
TTCAAAGGAA AGTGGAATGA ACCGTTTGAC GAAACATACACAAGGGAAAT
GCCCTTTAAA ATAAACCAGG AGGAGCAAAG GCCAGTGCAG ATGATGTATC
AGGAGGCCAC GTTTAAGCTC GCCCACGTGG GCGAGGTGCG CGCGCAGCTG
CTGGAGCTGCCCTACGCCAG GAAGGAGCTG AGCCTGCTGG TGCTGCTGCC
TGACGACGGC GTGGAGCTCAGCACGGTGGA AAAAAGTCTC ACTTTTGAGA
AACTCACAGC CTGGACCAAG CCAGACTGTATGAAGAGTAC TGAGGTTGAA
GTTCTCCTTC CAAAATTTAA ACTACAAGAG GATTATGACATGGAATCTGT
GCTTCGGCAT TTGGGAATTG TTGATGCCTT CCAACAGGGC AAGGCTGACT
TGTCGGCAAT GTCAGCGGAG AGAGACCTGT GTCTGTCCAA GTTCGTGCAC
AAGAGTTTTGTGGAGGTGAA TGAAGAAGGC ACCGAGGCAG CGGCAGCGTC
GAGCTGCTTT GTAGTTGCAGAGTGCTGCAT GGAATCTGGC CCCAGGTTCT
GTGCTGACCA CCCTTTCCTT TTCTTCATCAGGCACAACAG AGCCAACAGC
ATTCTGTTCT GTGGCAGGTT CTCATCGCCA TAA
SEQ ID No. 56
Serping1 variant 1 (Human) Protein
MASRLTLLTLLLLLLAGDRASSNPNATSSSSQDPESLQDRGEGKVATTVISKMLF
VEPILEVSSLPTTNSTTNSATKITANTTDEPTTQPTTEPTTQPTIQPTQPTTQLPTDSP
TQPTTGSFCPGPVTLCSDLESHSTEAVLGDALVDFSLKLYHAFSAMKKVETNMA
FSPFSIASLLTQVLLGAGENTKTNLESILSYPKDFTCVHQALKGFTTKGVTSVSQIF
HSPDLAIRDTFVNASRTLYSSSPRVLSNNSDANLELINTWVAKNTNNKISRLLDSL
PSDTRLVLLNAIYLSAKWKTTFDPKKTRMEPFHFKNSVIKVPMMNSKKYPVAH
FIDQTLKAKVGQLQLSHNLSLVILVPQNLKHRLEDMEQALSPSVFKAIMEKLEMS
KFQPTLLTLPRIKVTTSQDMLSIMEKLEFFDFSYDLNLCGLTEDPDLQVSAMQHQ
TVLELTETGVEAAAASAISVARTLLVFEVQQPFLFVLWDQQHKFPVFMGRVYDP
RA
SEQ ID No. 57
Serping1 variant 1 (Human) cDNA
ATGGCCTCC AGGCTGACCC TGCTGACCCT CCTGCTGCTG CTGCTGGCTG
GGGATAGAGC CTCCTCAAAT CCAAATGCTA CCAGCTCCAG CTCCCAGGAT
CCAGAGAGTTTGCAAGACAG AGGCGAAGGG AAGGTCGCAA CAACAGTTAT
CTCCAAGATG CTATTCGTTGAACCCATCCT GGAGGTTTCC AGCTTGCCGA
CAACCAACTC AACAACCAAT TCAGCCACCAAAATAACAGC TAATACCACT
GATGAACCCA CCACACAACC CACCACAGAG CCCACCACCCAACCCACCAT
CCAACCCACC CAACCAACTA CCCAGCTCCC AACAGATTCT CCTACCCAGC
CCACTACTGG GTCCTTCTGC CCAGGACCTG TTACTCTCTG CTCTGACTTG
GAGAGTCATTCAACAGAGGC CGTGTTGGGG GATGCTTTGG TAGATTTCTC
CCTGAAGCTC TACCACGCCTTCTCAGCAAT GAAGAAGGTG GAGACCAACA
TGGCCTTTTC CCCATTCAGC ATCGCCAGCCTCCTTACCCA GGTCCTGCTC
GGGGCTGGGG AGAACACCAA AACAAACCTG GAGAGCATCC
TCTCTTACCC CAAGGACTTC ACCTGTGTCC ACCAGGCCCT GAAGGGCTTC
ACGACCAAAGGTGTCACCTC AGTCTCTCAG ATCTTCCACA GCCCAGACCT
GGCCATAAGG GACACCTTTGTGAATGCCTC TCGGACCCTG TACAGCAGCA
GCCCCAGAGT CCTAAGCAAC AACAGTGACGCCAACTTGGA GCTCATCAAC
ACCTGGGTGG CCAAGAACAC CAACAACAAG ATCAGCCGGCTGCTAGACAG
TCTGCCCTCC GATACCCGCC TTGTCCTCCT CAATGCTATC TACCTGAGTG
CCAAGTGGAA GACAACATTT GATCCCAAGA AAACCAGAAT GGAACCCTTT
CACTTCAAAAACTCAGTTAT AAAAGTGCCC ATGATGAATA GCAAGAAGTA
CCCTGTGGCC CATTTCATTGACCAAACTTT GAAAGCCAAG GTGGGGCAGC
TGCAGCTCTC CCACAATCTG AGTTTGGTGATCCTGGTACC CCAGAACCTG
AAACATCGTC TTGAAGACAT GGAACAGGCT CTCAGCCCTTCTGTTTTCAA
GGCCATCATG GAGAAACTGG AGATGTCCAA GTTCCAGCCC ACTCTCCTAA
CACTACCCCG CATCAAAGTG ACGACCAGCC AGGATATGCT CTCAATCATG
GAGAAATTGGAATTCTTCGA TTTTTCTTAT GACCTTAACC TGTGTGGGCT
GACAGAGGAC CCAGATCTTCAGGTTTCTGC GATGCAGCAC CAGACAGTGC
TGGAACTGAC AGAGACTGGG GTGGAGGCGGCTGCAGCCTC CGCCATCTCT
GTGGCCCGCA CCCTGCTGGT CTTTGAAGTG CAGCAGCCCTTCCTCTTCGT
GCTCTGGGAC CAGCAGCACA AGTTCCCTGT CTTCATGGGG CGAGTATATG
ACCCCAGGGC CTGA
SEQ ID No. 58
Serping1 variant 2 (Human) Protein
MASRLTLLTLLLLLLAGDRASSNPNATSSSSQDPESLQDRGEGKVATTVISKMLF
VEPILEVSSLPTTNSTTNSATKITANTTDEPTTQPTTEPTTQPTIQPTQPTTQLPTDSP
TQPTTGSFCPGPVTLCSDLESHSTEAVLGDALVDFSLKLYHAFSAMKKVETNMA
FSPFSIASLLTQVLLGAGENTKTNLESILSYPKDFTCVHQALKGFTTKGVTSVSQIF
HSPDLAIRDTFVNASRTLYSSSPRVLSNNSDANLELINTWVAKNTNNKISRLLDSL
PSDTRLVLLNAIYLSAKWKTTFDPKKTRMEPFHFKNSVIKVPMMNSKKYPVAH
FIDQTLKAKVGQLQLSHNLSLVILVPQNLKHRLEDMEQALSPSVFKAIMEKLEMS
KFQPTLLTLPRIKVTTSQDMLSIMEKLEFFDFSYDLNLCGLTEDPDLQVSAMQHQ
TVLELTETGVEAAAASAISVARTLLVFEVQQPFLFVLWDQQHKFPVFMGRVYDP
RA
SEQ ID No. 59
Serping1 variant 2 (Human) cDNA
A TGGCCTCCAG GCTGACCCTGCTGACCCTCC TGCTGCTGCT GCTGGCTGGG
GATAGAGCCT CCTCAAATCC AAATGCTACCAGCTCCAGCT CCCAGGATCC
AGAGAGTTTG CAAGACAGAG GCGAAGGGAA GGTCGCAACAACAGTTATCT
CCAAGATGCT ATTCGTTGAA CCCATCCTGG AGGTTTCCAG CTTGCCGACA
ACCAACTCAA CAACCAATTC AGCCACCAAA ATAACAGCTA ATACCACTGA
TGAACCCACCACACAACCCA CCACAGAGCC CACCACCCAA CCCACCATCC
AACCCACCCA ACCAACTACCCAGCTCCCAA CAGATTCTCC TACCCAGCCC
ACTACTGGGT CCTTCTGCCC AGGACCTGTTACTCTCTGCT CTGACTTGGA
GAGTCATTCA ACAGAGGCCG TGTTGGGGGA TGCTTTGGTAGATTTCTCCC
TGAAGCTCTA CCACGCCTTC TCAGCAATGA AGAAGGTGGA GACCAACATG
GCCTTTTCCC CATTCAGCAT CGCCAGCCTC CTTACCCAGG TCCTGCTCGG
GGCTGGGGAGAACACCAAAA CAAACCTGGA GAGCATCCTC TCTTACCCCA
AGGACTTCAC CTGTGTCCACCAGGCCCTGA AGGGCTTCAC GACCAAAGGT
GTCACCTCAG TCTCTCAGAT CTTCCACAGCCCAGACCTGG CCATAAGGGA
CACCTTTGTG AATGCCTCTC GGACCCTGTA CAGCAGCAGCCCCAGAGTCC
TAAGCAACAA CAGTGACGCC AACTTGGAGC TCATCAACAC CTGGGTGGCC
AAGAACACCA ACAACAAGAT CAGCCGGCTG CTAGACAGTC TGCCCTCCGA
TACCCGCCTTGTCCTCCTCA ATGCTATCTA CCTGAGTGCC AAGTGGAAGA
CAACATTTGA TCCCAAGAAAACCAGAATGG AACCCTTTCA CTTCAAAAAC
TCAGTTATAA AAGTGCCCAT GATGAATAGCAAGAAGTACC CTGTGGCCCA
TTTCATTGAC CAAACTTTGA AAGCCAAGGT GGGGCAGCTGCAGCTCTCCC
ACAATCTGAG TTTGGTGATC CTGGTACCCC AGAACCTGAA ACATCGTCTT
GAAGACATGG AACAGGCTCT CAGCCCTTCT GTTTTCAAGG CCATCATGGA
GAAACTGGAGATGTCCAAGT TCCAGCCCAC TCTCCTAACA CTACCCCGCA
TCAAAGTGAC GACCAGCCAGGATATGCTCT CAATCATGGA GAAATTGGAA
TTCTTCGATT TTTCTTATGA CCTTAACCTGTGTGGGCTGA CAGAGGACCC
AGATCTTCAG GTTTCTGCGA TGCAGCACCA GACAGTGCTGGAACTGACAG
AGACTGGGGT GGAGGCGGCT GCAGCCTCCG CCATCTCTGT GGCCCGCACC
CTGCTGGTCT TTGAAGTGCA GCAGCCCTTC CTCTTCGTGC TCTGGGACCA
GCAGCACAAGTTCCCTGTCT TCATGGGGCG AGTATATGAC CCCAGGGCCT
GA
SEQ ID No. 60
Serpini1 variant 1 (Human) Protein
MAFLGLFSLLVLQSMATGATFPEEAIADLSVNMYNRLRATGEDENILFSPLSIALA
MGMMELGAQGSTQKEIRHSMGYDSLKNGEEFSFLKEFSNMVTAKESQYVMKIA
NSLFVQNGFHVNEEFLQMMKKYFNAAVNHVDFSQNVAVANYINKWVENNTN
NLVKDLVSPRDFDAATYLALINAVYFKGNWKSQFRPENTRTFSFTKDDESEVQIP
MMYQQGEFYYGEFSDGSNEAGGIYQVLEIPYEGDEISMMLVLSRQEVPLATLEPL
VKAQLVEEWANSVKKQKVEVYLPRFTVEQEIDLKDVLKALGITEIFIKDANLTGL
SDNKEIFLSKAIHKSFLEVNEEGSEAAAVSGMIAISRMAVLYPQVIVDHPFFFLIRN
RRTGTILFMGRVMHPETMNTSGHDFEEL
SEQ ID No. 61
Serpini1 variant 1 (Human) cDNA
ATGGCTTTC CTTGGACTCT TCTCTTTGCT GGTTCTGCAA AGTATGGCTA
CAGGGGCCAC TTTCCCTGAG GAAGCCATTG CTGACTTGTC AGTGAATATG
TATAATCGTCTTAGAGCCAC TGGTGAAGAT GAAAATATTC TCTTCTCTCC
ATTGAGTATT GCTCTTGCAATGGGAATGAT GGAACTTGGG GCCCAAGGAT
CTACCCAGAA AGAAATCCGC CACTCAATGGGATATGACAG CCTAAAAAAT
GGTGAAGAAT TTTCTTTCTT GAAGGAGTTT TCAAACATGGTAACTGCTAA
AGAGAGCCAA TATGTGATGA AAATTGCCAA TTCCTTGTTT GTGCAAAATG
GATTTCATGT CAATGAGGAG TTTTTGCAAA TGATGAAAAA ATATTTTAAT
GCAGCAGTAAATCATGTGGA CTTCAGTCAA AATGTAGCCG TGGCCAACTA
CATCAATAAG TGGGTGGAGAATAACACAAA CAATCTGGTG AAAGATTTGG
TATCCCCAAG GGATTTTGAT GCTGCCACTTATCTGGCCCT CATTAATGCT
GTCTATTTCA AGGGGAACTG GAAGTCGCAG TTTAGGCCTGAAAATACTAG
AACCTTTTCT TTCACTAAAG ATGATGAAAG TGAAGTCCAA ATTCCAATGA
TGTATCAGCA AGGAGAATTT TATTATGGGG AATTTAGTGA TGGCTCCAAT
GAAGCTGGTGGTATCTACCA AGTCCTAGAA ATACCATATG AAGGAGATGA
AATAAGCATG ATGCTGGTGCTGTCCAGACA GGAAGTTCCT CTTGCTACTC
TGGAGCCATT AGTCAAAGCA CAGCTGGTTGAAGAATGGGC AAACTCTGTG
AAGAAGCAAA AAGTAGAAGT ATACCTGCCC AGGTTCACAGTGGAACAGGA
AATTGATTTA AAAGATGTTT TGAAGGCTCT TGGAATAACT GAAATTTTCA
TCAAAGATGC AAATTTGACA GGCCTCTCTG ATAATAAGGA GATTTTTCTT
TCCAAAGCAATTCACAAGTC CTTCCTAGAG GTTAATGAAG AAGGCTCAGA
AGCTGCTGCT GTCTCAGGAATGATTGCAAT TAGTAGGATG GCTGTGCTGT
ATCCTCAAGT TATTGTCGAC CATCCATTTTTCTTTCTTAT CAGAAACAGG
AGAACTGGTA CAATTCTATT CATGGGACGA GTCATGCATCCTGAAACAAT
GAACACAAGT GGACATGATT TCGAAGAACT TTAA
SEQ ID No. 62
Serpini1 variant 2 (Human) Protein
MAFLGLFSLLVLQSMATGATFPEEAIADLSVNMYNRLRATGEDENILFSPLSIALA
MGMMELGAQGSTQKEIRHSMGYDSLKNGEEFSFLKEFSNMVTAKESQYVMKIA
NSLFVQNGFHVNEEFLQMMKKYFNAAVNHVDFSQNVAVANYINKWVENNTN
NLVKDLVSPRDFDAATYLALINAVYFKGNWKSQFRPENTRTFSFTKDDESEVQIP
MMYQQGEFYYGEFSDGSNEAGGIYQVLEIPYEGDEISMMLVLSRQEVPLATLEPL
VKAQLVEEWANSVKKQKVEVYLPRFTVEQEIDLKDVLKALGITEIFIKDANLTGL
SDNKEIFLSKAIHKSFLEVNEEGSEAAAVSGMIAISRMAVLYPQVIVDHPFFFLIRN
RRTGTILFMGRVMHPETMNTSGHDFEEL
SEQ ID No. 63
Serpini1 variant 2 (Human) cDNA
AT GGCTTTCCTT GGACTCTTCT CTTTGCTGGT TCTGCAAAGTATGGCTACAG
GGGCCACTTT CCCTGAGGAA GCCATTGCTG ACTTGTCAGT GAATATGTAT
AATCGTCTTA GAGCCACTGG TGAAGATGAA AATATTCTCT TCTCTCCATT
GAGTATTGCTCTTGCAATGG GAATGATGGA ACTTGGGGCC CAAGGATCTA
CCCAGAAAGA AATCCGCCACTCAATGGGAT ATGACAGCCT AAAAAATGGT
GAAGAATTTT CTTTCTTGAA GGAGTTTTCAAACATGGTAA CTGCTAAAGA
GAGCCAATAT GTGATGAAAA TTGCCAATTC CTTGTTTGTGCAAAATGGAT
TTCATGTCAA TGAGGAGTTT TTGCAAATGA TGAAAAAATA TTTTAATGCA
GCAGTAAATC ATGTGGACTT CAGTCAAAAT GTAGCCGTGG CCAACTACAT
CAATAAGTGGGTGGAGAATA ACACAAACAA TCTGGTGAAA GATTTGGTAT
CCCCAAGGGA TTTTGATGCTGCCACTTATC TGGCCCTCAT TAATGCTGTC
TATTTCAAGG GGAACTGGAA GTCGCAGTTTAGGCCTGAAA ATACTAGAAC
CTTTTCTTTC ACTAAAGATG ATGAAAGTGA AGTCCAAATTCCAATGATGT
ATCAGCAAGG AGAATTTTAT TATGGGGAAT TTAGTGATGG CTCCAATGAA
GCTGGTGGTA TCTACCAAGT CCTAGAAATA CCATATGAAG GAGATGAAAT
AAGCATGATGCTGGTGCTGT CCAGACAGGA AGTTCCTCTT GCTACTCTGG
AGCCATTAGT CAAAGCACAGCTGGTTGAAG AATGGGCAAA CTCTGTGAAG
AAGCAAAAAG TAGAAGTATA CCTGCCCAGGTTCACAGTGG AACAGGAAAT
TGATTTAAAA GATGTTTTGA AGGCTCTTGG AATAACTGAAATTTTCATCA
AAGATGCAAA TTTGACAGGC CTCTCTGATA ATAAGGAGAT
TTTTCTTTCCAAAGCAATTC ACAAGTCCTT CCTAGAGGTT AATGAAGAAG
GCTCAGAAGC TGCTGCTGTCTCAGGAATGA TTGCAATTAG TAGGATGGCT
GTGCTGTATC CTCAAGTTAT TGTCGACCATCCATTTTTCT TTCTTATCAG
AAACAGGAGA ACTGGTACAA TTCTATTCAT GGGACGAGTCATGCATCCTG
AAACAATGAA CACAAGTGGA CATGATTTCG AAGAACTTTA A

All publications and patent documents disclosed or referred to herein are incorporated by reference in their entirety. The foregoing description has been presented only for purposes of illustration and description. This description is not intended to limit the invention to the precise form disclosed. It is intended that the scope of the invention be defined by the claims appended hereto.

1. A method of treating an inflammatory condition in a subject, comprising administering a serpin protein and administering methylprednisolone.

2. The method of claim 1, wherein said inflammatory condition is neuromyelitis optica (NMO).

3. The method of claim 1, wherein said inflammatory condition is multiple sclerosis (MS).

4. The method of claim 3, wherein said MS is progressive.

5. The method of claim 1, wherein said inflammatory condition is Amyotrophic lateral sclerosis (ALS).

6. The method of claim 1, wherein said serpin protein has at least a 90% sequence identity to SEQ ID NO:1 and has alpha-1 antitrypsin (A1AT) activity.

7. The method of claim 6, wherein said serpin protein has the sequence of SEQ ID NO:1.

8. The method of claim 1, wherein said serpin protein is encoded by a nucleic acid that has at least a 90% sequence identity to SEQ ID NO:2 and wherein said serpin protein has alpha-1 antitrypsin (A1AT) activity.

9. The method of claim 8 wherein said serpin protein is encoded by a nucleic acid that has the sequence of SEQ ID NO:2.

10. The method of claim 1, wherein said serpin protein and methylprednisolone are administered simultaneously.

11. A method of treating an inflammatory condition in a subject, comprising administering a nucleic acid that encodes a serpin protein and administering methylprednisolone.

12. The method of claim 11, wherein said inflammatory condition is neuromyelitis optica (NMO).

13. The method of claim 11, wherein said inflammatory condition is multiple sclerosis (MS).

14. The method of claim 13, wherein said MS is progressive.

15. The method of claim 11, wherein said inflammatory condition is Amyotrophic lateral sclerosis (ALS).

16. The method of claim 11, wherein said nucleic acid encodes a protein having at least a 90% sequence identity to SEQ ID NO:1 and has alpha-1 antitrypsin (A1AT) activity.

17. The method of claim 16, wherein said nucleic acid encodes a protein having the sequence of SEQ ID NO:1.

18. The method of claim 11, wherein said nucleic acid that has at least a 90% sequence identity to SEQ ID NO:2 and wherein said serpin protein has alpha-1 antitrypsin (A1AT) activity.

19. The method of claim 18, wherein said nucleic acid has the sequence of SEQ ID NO:2.

20. The method of claim 11, wherein said nucleic acid is administered by a route selected from the group consisting of transfected autologous patient cells, viral vectors, naked nucleic acid preparations, homologous recombination, knock-in, zinc finger nucleases (ZFNs), transcription activator-like effector nucleases (TALENs), and clustered regularly interspaced short palindromic repeats)/Cas9 (CRISPR).

21. A method of treating an inflammatory condition in a subject, comprising increasing the expression of an endogenous serpin protein and administering methylprednisolone.

22. The method of claim 21, wherein said inflammatory condition is neuromyelitis optica (NMO).

23. The method of claim 21, wherein said inflammatory condition is multiple sclerosis (MS).

24. The method of claim 23, wherein said MS is progressive.

25. The method of claim 21, wherein said inflammatory condition is Amyotrophic lateral sclerosis (ALS).

26. The method of claim 21, wherein said endogenous serpin protein has at least a 90% sequence identity to SEQ ID NO:1 and has alpha-1 antitrypsin (A1AT) activity.

27. The method of claim 21, wherein said endogenous serpin protein has the sequence of SEQ ID NO:1.

28. The method of claim 21, wherein said endogenous serpin protein is encoded by a nucleic acid that has at least a 90% sequence identity to SEQ ID NO:2 and wherein said serpin protein has alpha-1 antitrypsin (A1AT) activity.

29. The method of claim 28, wherein said endogenous serpin protein is encoded by a nucleic acid that has the sequence of SEQ ID NO:2.

30. The method of claim 21, wherein said increase in said serpin expression is accomplished using a technology selected from the group consisting of zinc finger nucleases (ZFNs), transcription activator-like effector nucleases (TALENs), and clustered regularly interspaced short palindromic repeats (CRISPR)/Cas9.

31. A pharmaceutical composition, comprising a serpin protein and methylprednisolone.

32. The pharmaceutical composition of claim 31, wherein said serpin protein has at least a 90% sequence identity to SEQ ID NO:1 and has alpha-1 antitrypsin (A1AT) activity.

33. The pharmaceutical composition of claim 32, wherein said serpin protein has the sequence of SEQ ID NO:1.

34. The pharmaceutical composition of claim 31, wherein said serpin protein is encoded by a nucleic acid that has at least a 90% sequence identity to SEQ ID NO:2 and wherein said serpin protein has alpha-1 antitrypsin (A1AT) activity.

35. The pharmaceutical composition of claim 34, wherein said serpin protein is encoded by a nucleic acid that has the sequence of SEQ ID NO:2.
